SEQUENCE LISTING (250 sequences)

SEQ ID NO: 1 - protein, 19 aa, Artificial Sequence
  Synthetic Construct
  MOD_RES (10)..(10): Xaa is N epsilon -protected lysine
  MOD_RES (15)..(15): Xaa is N epsilon -protected lysine
TFVYGGCRAXRNNFXSAED

SEQ ID NO: 2 - protein, 19 aa, Artificial Sequence
  Synthetic Construct
  MOD_RES (18)..(18): Xaa is N epsilon -protected lysine
TFQYGGCMGNGNNFVTEXE

SEQ ID NO: 3 - protein, 19 aa, Artificial Sequence
  Synthetic Construct
PFFYGGCGGNRNNFDTEEY

SEQ ID NO: 4 - protein, 19 aa, Artificial Sequence
  Synthetic Construct
  MOD_RES (11)..(11): Xaa is N epsilon -protected lysine
SFYYGGCLGNXNNYLREEE

SEQ ID NO: 5 - protein, 19 aa, Artificial Sequence
  Synthetic Construct
  MOD_RES (10)..(10): Xaa is N epsilon -protected lysine
  MOD_RES (15)..(15): Xaa is N epsilon -protected lysine
  MOD_RES (18)..(18): Xaa is N epsilon -protected lysine
TFFYGGCRAXRNNFXRAXY

SEQ ID NO: 6 - protein, 19 aa, Artificial Sequence
  Synthetic Construct
  MOD_RES (10)..(10): Xaa is N epsilon -protected lysine
  MOD_RES (15)..(15): Xaa is N epsilon -protected lysine
  MOD_RES (18)..(18): Xaa is N epsilon -protected lysine
TFFYGGCRGXRNNFXRAXY

SEQ ID NO: 7 - protein, 19 aa, Artificial Sequence
  Synthetic Construct
  MOD_RES (10)..(11): Xaa is N epsilon -protected lysine
  MOD_RES (15)..(15): Xaa is N epsilon -protected lysine
  MOD_RES (18)..(18): Xaa is N epsilon -protected lysine
TFFYGGCRAXXNNYXRAXY

SEQ ID NO: 8 - protein, 19 aa, Artificial Sequence
  Synthetic Construct
  MOD_RES (10)..(11): Xaa is N epsilon -protected lysine
  MOD_RES (15)..(15): Xaa is N epsilon -protected lysine
  MOD_RES (18)..(18): Xaa is N epsilon -protected lysine
TFFYGGCRGXXNNFXRAXY

SEQ ID NO: 9 - protein, 19 aa, Artificial Sequence
  Synthetic Construct
  MOD_RES (10)..(10): Xaa is N epsilon -protected lysine
  MOD_RES (15)..(15): Xaa is N epsilon -protected lysine
  MOD_RES (18)..(18): Xaa is N epsilon -protected lysine
TFQYGGCRAXRNNFXRAXY

SEQ ID NO: 10 - protein, 19 aa, Artificial Sequence
  Synthetic Construct
  MOD_RES (10)..(11): Xaa is N epsilon -protected lysine
  MOD_RES (15)..(15): Xaa is N epsilon -protected lysine
  MOD_RES (18)..(18): Xaa is N epsilon -protected lysine
TFQYGGCRGXXNNFXRAXY

SEQ ID NO: 11 - protein, 19 aa, Artificial Sequence
  Synthetic Construct
  MOD_RES (10)..(10): Xaa is N epsilon -protected lysine
  MOD_RES (15)..(15): Xaa is N epsilon -protected lysine
  MOD_RES (18)..(18): Xaa is N epsilon -protected lysine
TFFYGGCLGXRNNFXRAXY

SEQ ID NO: 12 - protein, 19 aa, Artificial Sequence
  Synthetic Construct
  MOD_RES (10)..(10): Xaa is N epsilon -protected lysine
  MOD_RES (15)..(15): Xaa is N epsilon -protected lysine
  MOD_RES (18)..(18): Xaa is N epsilon -protected lysine
TFFYGGSLGXRNNFXRAXY

SEQ ID NO: 13 - protein, 19 aa, Artificial Sequence
  Synthetic Construct
  MOD_RES (10)..(11): Xaa is N epsilon -protected lysine
  MOD_RES (15)..(15): Xaa is N epsilon -protected lysine
  MOD_RES (18)..(18): Xaa is N epsilon -protected lysine
PFFYGGCGGXXNNFXRAXY

SEQ ID NO: 14 - protein, 19 aa, Artificial Sequence
  Synthetic Construct
  MOD_RES (10)..(10): Xaa is N epsilon -protected lysine
  MOD_RES (15)..(15): Xaa is N epsilon -protected lysine
  MOD_RES (18)..(18): Xaa is N epsilon -protected lysine
TFFYGGCRGXGNNYXRAXY

SEQ ID NO: 15 - protein, 19 aa, Artificial Sequence
  Synthetic Construct
  MOD_RES (10)..(10): Xaa is N epsilon -protected lysine
  MOD_RES (18)..(18): Xaa is N epsilon -protected lysine
PFFYGGCRGXRNNFLRAXY

SEQ ID NO: 16 - protein, 19 aa, Artificial Sequence
  Synthetic Construct
  MOD_RES (10)..(10): Xaa is N epsilon -protected lysine
  MOD_RES (15)..(15): Xaa is N epsilon -protected lysine
  MOD_RES (18)..(18): Xaa is N epsilon -protected lysine
TFFYGGCRGXRNNFXREXY

SEQ ID NO: 17 - protein, 19 aa, Artificial Sequence
  Synthetic Construct
  MOD_RES (10)..(11): Xaa is N epsilon -protected lysine
  MOD_RES (15)..(15): Xaa is N epsilon -protected lysine
  MOD_RES (18)..(18): Xaa is N epsilon -protected lysine
PFFYGGCRAXXNNFXRAXE

SEQ ID NO: 18 - protein, 19 aa, Artificial Sequence
  Synthetic Construct
  MOD_RES (10)..(10): Xaa is N epsilon -protected lysine
  MOD_RES (15)..(15): Xaa is N epsilon -protected lysine
  MOD_RES (18)..(18): Xaa is N epsilon -protected lysine
TFFYGGCRGXRNNFXRAXD

SEQ ID NO: 19 - protein, 19 aa, Artificial Sequence
  Synthetic Construct
  MOD_RES (10)..(10): Xaa is N epsilon -protected lysine
  MOD_RES (18)..(18): Xaa is N epsilon -protected lysine
TFFYGGCRAXRNNFDRAXY

SEQ ID NO: 20 - protein, 19 aa, Artificial Sequence
  Synthetic Construct
  MOD_RES (10)..(11): Xaa is N epsilon -protected lysine
  MOD_RES (15)..(15): Xaa is N epsilon -protected lysine
TFFYGGCRGXXNNFXRAEY

SEQ ID NO: 21 - protein, 19 aa, Artificial Sequence
  Synthetic Construct
  MOD_RES (15)..(15): Xaa is N epsilon -protected lysine
  MOD_RES (18)..(18): Xaa is N epsilon -protected lysine
PFFYGGCGANRNNFXRAXY

SEQ ID NO: 22 - protein, 19 aa, Artificial Sequence
  Synthetic Construct
  MOD_RES (10)..(11): Xaa is N epsilon -protected lysine
  MOD_RES (15)..(15): Xaa is N epsilon -protected lysine
  MOD_RES (18)..(18): Xaa is N epsilon -protected lysine
TFFYGGCGGXXNNFXTAXY

SEQ ID NO: 23 - protein, 19 aa, Artificial Sequence
  Synthetic Construct
  MOD_RES (18)..(18): Xaa is N epsilon -protected lysine
TFFYGGCRGNRNNFLRAXY

SEQ ID NO: 24 - protein, 19 aa, Artificial Sequence
  Synthetic Construct
  MOD_RES (15)..(15): Xaa is N epsilon -protected lysine
  MOD_RES (18)..(18): Xaa is N epsilon -protected lysine
TFFYGGCRGNRNNFXTAXY

SEQ ID NO: 25 - protein, 19 aa, Artificial Sequence
  Synthetic Construct
  MOD_RES (15)..(15): Xaa is N epsilon -protected lysine
  MOD_RES (18)..(18): Xaa is N epsilon -protected lysine
TFFYGGSRGNRNNFXTAXY

SEQ ID NO: 26 - protein, 19 aa, Artificial Sequence
  Synthetic Construct
  MOD_RES (15)..(15): Xaa is N epsilon -protected lysine
  MOD_RES (18)..(18): Xaa is N epsilon -protected lysine
TFFYGGCLGNGNNFXRAXY

SEQ ID NO: 27 - protein, 19 aa, Artificial Sequence
  Synthetic Construct
  MOD_RES (18)..(18): Xaa is N epsilon -protected lysine
TFFYGGCLGNRNNFLRAXY

SEQ ID NO: 28 - protein, 19 aa, Artificial Sequence
  Synthetic Construct
  MOD_RES (15)..(15): Xaa is N epsilon -protected lysine
  MOD_RES (18)..(18): Xaa is N epsilon -protected lysine
TFFYGGCLGNRNNFXTAXY

SEQ ID NO: 29 - protein, 19 aa, Artificial Sequence
  Synthetic Construct
  MOD_RES (15)..(15): Xaa is N epsilon -protected lysine
  MOD_RES (18)..(18): Xaa is N epsilon -protected lysine
TFFYGGCRGNGNNFXSAXY

SEQ ID NO: 30 - protein, 19 aa, Artificial Sequence
  Synthetic Construct
  MOD_RES (10)..(11): Xaa is N epsilon -protected lysine
  MOD_RES (18)..(18): Xaa is N epsilon -protected lysine
TFFYGGCRGXXNNFDREXY

SEQ ID NO: 31 - protein, 19 aa, Artificial Sequence
  Synthetic Construct
  MOD_RES (10)..(10): Xaa is N epsilon -protected lysine
  MOD_RES (18)..(18): Xaa is N epsilon -protected lysine
TFFYGGCRGXRNNFLREXE

SEQ ID NO: 32 - protein, 19 aa, Artificial Sequence
  Synthetic Construct
  MOD_RES (10)..(10): Xaa is N epsilon -protected lysine
  MOD_RES (18)..(18): Xaa is N epsilon -protected lysine
TFFYGGCRGXGNNFDRAXY

SEQ ID NO: 33 - protein, 19 aa, Artificial Sequence
  Synthetic Construct
  MOD_RES (10)..(10): Xaa is N epsilon -protected lysine
  MOD_RES (18)..(18): Xaa is N epsilon -protected lysine
TFFYGGSRGXGNNFDRAXY

SEQ ID NO: 34 - protein, 19 aa, Artificial Sequence
  Synthetic Construct
  MOD_RES (18)..(18): Xaa is N epsilon -protected lysine
TFFYGGCRGNGNNFVTAXY

SEQ ID NO: 35 - protein, 19 aa, Artificial Sequence
  Synthetic Construct
  MOD_RES (10)..(10): Xaa is N epsilon -protected lysine
  MOD_RES (18)..(18): Xaa is N epsilon -protected lysine
PFFYGGCGGXGNNYVTAXY

SEQ ID NO: 36 - protein, 19 aa, Artificial Sequence
  Synthetic Construct
  MOD_RES (10)..(10): Xaa is N epsilon -protected lysine
  MOD_RES (18)..(18): Xaa is N epsilon -protected lysine
TFFYGGCLGXGNNFLTAXY

SEQ ID NO: 37 - protein, 19 aa, Artificial Sequence
  Synthetic Construct
  MOD_RES (11)..(11): Xaa is N epsilon -protected lysine
  MOD_RES (18)..(18): Xaa is N epsilon -protected lysine
SFFYGGCLGNXNNFLTAXY

SEQ ID NO: 38 - protein, 19 aa, Artificial Sequence
  Synthetic Construct
  MOD_RES (11)..(11): Xaa is N epsilon -protected lysine
  MOD_RES (18)..(18): Xaa is N epsilon -protected lysine
TFFYGGCGGNXNNFVREXY

SEQ ID NO: 39 - protein, 19 aa, Artificial Sequence
  Synthetic Construct
  MOD_RES (11)..(11): Xaa is N epsilon -protected lysine
  MOD_RES (18)..(18): Xaa is N epsilon -protected lysine
TFFYGGCMGNXNNFVREXY

SEQ ID NO: 40 - protein, 19 aa, Artificial Sequence
  Synthetic Construct
  MOD_RES (11)..(11): Xaa is N epsilon -protected lysine
  MOD_RES (18)..(18): Xaa is N epsilon -protected lysine
TFFYGGSMGNXNNFVREXY

SEQ ID NO: 41 - protein, 19 aa, Artificial Sequence
  Synthetic Construct
  MOD_RES (18)..(18): Xaa is N epsilon -protected lysine
PFFYGGCLGNRNNYVREXY

SEQ ID NO: 42 - protein, 19 aa, Artificial Sequence
  Synthetic Construct
  MOD_RES (18)..(18): Xaa is N epsilon -protected lysine
TFFYGGCLGNRNNFVREXY

SEQ ID NO: 43 - protein, 19 aa, Artificial Sequence
  Synthetic Construct
  MOD_RES (11)..(11): Xaa is N epsilon -protected lysine
  MOD_RES (18)..(18): Xaa is N epsilon -protected lysine
TFFYGGCLGNXNNYVREXY

SEQ ID NO: 44 - protein, 19 aa, Artificial Sequence
  Synthetic Construct
  MOD_RES (18)..(18): Xaa is N epsilon -protected lysine
TFFYGGCGGNGNNFLTAXY

SEQ ID NO: 45 - protein, 19 aa, Artificial Sequence
  Synthetic Construct
TFFYGGCRGNRNNFLTAEY

SEQ ID NO: 46 - protein, 19 aa, Artificial Sequence
  Synthetic Construct
  MOD_RES (15)..(15): Xaa is N epsilon -protected lysine
TFFYGGCRGNGNNFXSAEY

SEQ ID NO: 47 - protein, 19 aa, Artificial Sequence
  Synthetic Construct
  MOD_RES (11)..(11): Xaa is N epsilon -protected lysine
  MOD_RES (15)..(15): Xaa is N epsilon -protected lysine
PFFYGGCLGNXNNFXTAEY

SEQ ID NO: 48 - protein, 19 aa, Artificial Sequence
  Synthetic Construct
  MOD_RES (15)..(15): Xaa is N epsilon -protected lysine
TFFYGGCRGNRNNFXTEEY

SEQ ID NO: 49 - protein, 19 aa, Artificial Sequence
  Synthetic Construct
  MOD_RES (10)..(10): Xaa is N epsilon -protected lysine
  MOD_RES (15)..(15): Xaa is N epsilon -protected lysine
TFFYGGCRGXRNNFXTEED

SEQ ID NO: 50 - protein, 19 aa, Artificial Sequence
  Synthetic Construct
  MOD_RES (18)..(18): Xaa is N epsilon -protected lysine
PFFYGGCGGNGNNFVREXY

SEQ ID NO: 51 - protein, 19 aa, Artificial Sequence
  Synthetic Construct
  MOD_RES (18)..(18): Xaa is N epsilon -protected lysine
SFFYGGCMGNGNNFVREXY

SEQ ID NO: 52 - protein, 19 aa, Artificial Sequence
  Synthetic Construct
  MOD_RES (18)..(18): Xaa is N epsilon -protected lysine
PFFYGGCGGNGNNFLREXY

SEQ ID NO: 53 - protein, 19 aa, Artificial Sequence
  Synthetic Construct
  MOD_RES (18)..(18): Xaa is N epsilon -protected lysine
TFFYGGCLGNGNNFVREXY

SEQ ID NO: 54 - protein, 19 aa, Artificial Sequence
  Synthetic Construct
  MOD_RES (18)..(18): Xaa is N epsilon -protected lysine
SFFYGGCLGNGNNYLREXY

SEQ ID NO: 55 - protein, 19 aa, Artificial Sequence
  Synthetic Construct
  MOD_RES (18)..(18): Xaa is N epsilon -protected lysine
TFFYGGSLGNGNNFVREXY

SEQ ID NO: 56 - protein, 19 aa, Artificial Sequence
  Synthetic Construct
TFFYGGCRGNGNNFVTAEY

SEQ ID NO: 57 - protein, 19 aa, Artificial Sequence
  Synthetic Construct
  MOD_RES (10)..(10): Xaa is N epsilon -protected lysine
TFFYGGCLGXGNNFVSAEY

SEQ ID NO: 58 - protein, 19 aa, Artificial Sequence
  Synthetic Construct
TFFYGGCLGNRNNFDRAEY

SEQ ID NO: 59 - protein, 19 aa, Artificial Sequence
  Synthetic Construct
TFFYGGCLGNRNNFLREEY

SEQ ID NO: 60 - protein, 19 aa, Artificial Sequence
  Synthetic Construct
  MOD_RES (11)..(11): Xaa is N epsilon -protected lysine
TFFYGGCLGNXNNYLREEY

SEQ ID NO: 61 - protein, 19 aa, Artificial Sequence
  Synthetic Construct
PFFYGGCGGNRNNYLREEY

SEQ ID NO: 62 - protein, 19 aa, Artificial Sequence
  Synthetic Construct
PFFYGGSGGNRNNYLREEY

SEQ ID NO: 63 - protein, 19 aa, Artificial Sequence
  Synthetic Construct
MRPDFCLEPPYTGPCVARI

SEQ ID NO: 64 - protein, 21 aa, Artificial Sequence
  Synthetic Construct
  MOD_RES (11)..(11): Xaa is N epsilon -protected lysine
ARIIRYFYNAXAGLCQTFVYG

SEQ ID NO: 65 - protein, 22 aa, Artificial Sequence
  Synthetic Construct
  MOD_RES (7)..(7): Xaa is N epsilon -protected lysine
  MOD_RES (12)..(12): Xaa is N epsilon -protected lysine
YGGCRAXRNNYXSAEDCMRTCG

SEQ ID NO: 66 - protein, 22 aa, Artificial Sequence
  Synthetic Construct
PDFCLEPPYTGPCVARIIRYFY

SEQ ID NO: 67 - protein, 19 aa, Artificial Sequence
  Synthetic Construct
  MOD_RES (10)..(10): Xaa is N epsilon -protected lysine
  MOD_RES (15)..(15): Xaa is N epsilon -protected lysine
TFFYGGCRGXRNNFXTEEY

SEQ ID NO: 68 - protein, 19 aa, Artificial Sequence
  Synthetic Construct
  MOD_RES (1)..(1): Xaa is N epsilon -protected lysine
  MOD_RES (10)..(10): Xaa is N epsilon -protected lysine
  MOD_RES (15)..(15): Xaa is N epsilon -protected lysine
XFFYGGCRGXRNNFXTEEY

SEQ ID NO: 69 - protein, 19 aa, Artificial Sequence
  Synthetic Construct
  MOD_RES (10)..(10): Xaa is N epsilon -protected lysine
  MOD_RES (15)..(15): Xaa is N epsilon -protected lysine
TFYYGGCRGXRNNYXTEEY

SEQ ID NO: 70 - skipped

SEQ ID NO: 71 - protein, 20 aa, Artificial Sequence
  Synthetic Construct
  MOD_RES (11)..(11): Xaa is N epsilon -protected lysine
  MOD_RES (16)..(16): Xaa is N epsilon -protected lysine
CTFFYGCCRGXRNNFXTEEY

SEQ ID NO: 72 - protein, 20 aa, Artificial Sequence
  Synthetic Construct
  MOD_RES (10)..(10): Xaa is N epsilon -protected lysine
  MOD_RES (15)..(15): Xaa is N epsilon -protected lysine
TFFYGGCRGXRNNFXTEEYC

SEQ ID NO: 73 - protein, 20 aa, Artificial Sequence
  Synthetic Construct
  MOD_RES (11)..(11): Xaa is N epsilon -protected lysine
  MOD_RES (16)..(16): Xaa is N epsilon -protected lysine
CTFFYGSCRGXRNNFXTEEY

SEQ ID NO: 74 - skipped

SEQ ID NO: 75 - protein, 19 aa, Artificial Sequence
  Synthetic Construct
  MOD_RES (10)..(10): Xaa is N epsilon -protected lysine
  MOD_RES (15)..(15): Xaa is N epsilon -protected lysine
PFFYGGCRGXRNNFXTEEY

SEQ ID NO: 76 - protein, 19 aa, Artificial Sequence
  Synthetic Construct
  MOD_RES (10)..(10): Xaa is N epsilon -protected lysine
  MOD_RES (15)..(15): Xaa is N epsilon -protected lysine
  MOD_RES (17)..(17)
TFFYGGCRGXRNNFXTXEY

SEQ ID NO: 77 - protein, 19 aa, Artificial Sequence
  Synthetic Construct
  MOD_RES (7)..(7): Xaa is N epsilon -protected lysine
  MOD_RES (10)..(10): Xaa is N epsilon -protected lysine
  MOD_RES (15)..(15): Xaa is N epsilon -protected lysine
TFFYGGXRGXRNNFXTEEY

SEQ ID NO: 78 - protein, 19 aa, Artificial Sequence
  Synthetic Construct
  MOD_RES (10)..(10): Xaa is N epsilon -protected lysine
  MOD_RES (15)..(15): Xaa is N epsilon -protected lysine
  MOD_RES (17)..(17): Xaa is N epsilon -protected lysine
TFFYGGCRGXRNNFXTXRY

SEQ ID NO: 79 - protein, 19 aa, Artificial Sequence
  Synthetic Construct
  MOD_RES (7)..(7): Xaa is N epsilon -protected lysine
  MOD_RES (10)..(10): Xaa is N epsilon -protected lysine
  MOD_RES (15)..(15): Xaa is N epsilon -protected lysine
TFFYGGXRGXRNNFXTAEY

SEQ ID NO: 80 - protein, 19 aa, Artificial Sequence
  Synthetic Construct
  MOD_RES (7)..(7): Xaa is N -epsilon protected lysine
  MOD_RES (10)..(10): Xaa is N -epsilon protected lysine
  MOD_RES (15)..(15): Xaa is N -epsilon protected lysine
TFFYGGXRGXRNNFXTAGY

SEQ ID NO: 81 - protein, 19 aa, Artificial Sequence
  Synthetic Construct
  MOD_RES (7)..(7): Xaa is N epsilon -protected lysine
  MOD_RES (10)..(10): Xaa is N epsilon -protected lysine
  MOD_RES (15)..(15): Xaa is N epsilon -protected lysine
  MOD_RES (18)..(18): Xaa is N epsilon -protected lysine
TFFYGGXRGXRNNFXREXY

SEQ ID NO: 82 - protein, 19 aa, Artificial Sequence
  Synthetic Construct
  MOD_RES (7)..(7): Xaa is N epsilon -protected lysine
  MOD_RES (10)..(10): Xaa is N epsilon -protected lysine
  MOD_RES (15)..(15): Xaa is N epsilon -protected lysine
  MOD_RES (18)..(18): Xaa is N epsilon -protected lysine
TFFYGGXRGXRNNFXRAXY

SEQ ID NO: 83 - protein, 19 aa, Artificial Sequence
  Synthetic Construct
  MOD_RES (15)..(15): Xaa is N epsilon -protected lysine
TFFYGGCLGNRNNFXTEEY

SEQ ID NO: 84 - protein, 19 aa, Artificial Sequence
  Sythetic Construct
  MOD_RES (10)..(10): Xaa is N epsilon -protected lysine
  MOD_RES (15)..(15): Xaa is N epsilon -protected lysine
TFFYGCGRGXRNNFXTEEY

SEQ ID NO: 85 - protein, 19 aa, Artificial Sequence
  Synthetic Construct
  MOD_RES (10)..(10): Xaa is N epsilon -protected lysine
  MOD_RES (15)..(15): Xaa is N epsilon -protected lysine
TFFYGGRCGXRNNFXTEEY

SEQ ID NO: 86 - protein, 19 aa, Artificial Sequence
  Synthetic Construct
TFFYGGCLGNGNNFDTEEE

SEQ ID NO: 87 - protein, 19 aa, Artificial Sequence
  Synthetic Construct
  MOD_RES (10)..(10): Xaa is N epsilon -protected lysine
  MOD_RES (15)..(15): Xaa is N epsilon -protected lysine
TFQYGGCRGXRNNFXTEEY

SEQ ID NO: 88 - protein, 19 aa, Artificial Sequence
  Synthetic Construct
  MOD_RES (3)..(3): Xaa is N epsilon -protected lysine
  MOD_RES (11)..(11): Xaa is N epsilon -protected lysine
YNXEFGTFNTXGCERGYRF

SEQ ID NO: 89 - protein, 19 aa, Artificial Sequence
  Synthetic Construct
  MOD_RES (3)..(3): Xaa is N epsilon -protected lysine
RFXYGGCLGNMNNFETLEE

SEQ ID NO: 90 - protein, 19 aa, Artificial Sequence
  Synthetic Construct
  MOD_RES (3)..(3): Xaa is N epsilon -protected lysine
  MOD_RES (11)..(11): Xaa is N epsilon -protected lysine
  MOD_RES (18)..(18): Xaa is N epsilon -protected lysine
RFXYGGCLGNXNNFLRLXY

SEQ ID NO: 91 - protein, 19 aa, Artificial Sequence
  Synthetic Construct
  MOD_RES (3)..(3): Xaa is N epsilon -protected lysine
  MOD_RES (11)..(11): Xaa is N epsilon -protected lysine
  MOD_RES (18)..(18): Xaa is N epsilon -protected lysine
RFXYGGCLGNXNNYLRLXY

SEQ ID NO: 92 - protein, 22 aa, Artificial Sequence
  Synthetic Construct
  MOD_RES (1)..(1): Xaa is N epsilon -protected lysine
  MOD_RES (3)..(3): Xaa is N epsilon -protected lysine
  MOD_RES (5)..(5): Xaa is N epsilon -protected lysine
  MOD_RES (7)..(8): Xaa is N epsilon -protected lysine
  MOD_RES (12)..(12): Xaa is N epsilon -protected lysine
  MOD_RES (20)..(20): Xaa is N epsilon -protected lysine
XTXRXRXXQRVXIAYEEIFXNY

SEQ ID NO: 93 - protein, 15 aa, Artificial Sequence
  Synthetic Construct
  MOD_RES (1)..(1): Xaa is N epsilon -protected lysine
  MOD_RES (3)..(3): Xaa is N epsilon -protected lysine
  MOD_RES (5)..(5): Xaa is N epsilon -protected lysine
  MOD_RES (7)..(8): Xaa is N epsilon -protected lysine
  MOD_RES (12)..(12): Xaa is N epsilon -protected lysine
XTXRXRXXQRVXIAY

SEQ ID NO: 94 - protein, 17 aa, Artificial Sequence
  Synthetic Construct
RGGRLSYSRRFSTSTGR

SEQ ID NO: 95 - protein, 10 aa, Artificial Sequence
  Synthetic Construct
RRLSYSRRRF

SEQ ID NO: 96 - protein, 16 aa, Artificial Sequence
  Synthetic Construct
  MOD_RES (4)..(4): Xaa is N epsilon -protected lysine
  MOD_RES (13)..(13): Xaa is N epsilon -protected lysine
  MOD_RES (15)..(16): Xaa is N epsilon -protected lysine
RQIXIWFQNRRMXWXX

SEQ ID NO: 97 - protein, 19 aa, Artificial Sequence
  Synthetic Construct
  MOD_RES (10)..(10): Xaa is N epsilon -protected lysine
  MOD_RES (15)..(15): Xaa is N epsilon -protected lysine
TFFYGGSRGXRNNFXTEEY

SEQ ID NO: 98 - protein, 59 aa, Artificial Sequence
  Synthetic Construct
  MOD_RES (27)..(27): Xaa is N epsilon -protected lysine
  MOD_RES (42)..(42): Xaa is N epsilon -protected lysine
  MOD_RES (47)..(47): Xaa is N epsilon -protected lysine
MRPDFCLEPPYTGPCVARIIRYFYNAXAGLCQTFVYGGCRAXRNNFXSAEDCMRTCGGA

SEQ ID NO: 99 - protein, 19 aa, Artificial Sequence
  Synthetic Construct
  MOD_RES (10)..(10): Xaa is N epsilon -protected lysine
  MOD_RES (15)..(15): Xaa is N epsilon -protected lysine
  MOD_RES (17)..(17): Xaa is N epsilon -protected lysine
TFFYGGCRGXRNNFXTXEY

SEQ ID NO: 100 - protein, 19 aa, Artificial Sequence
  Synthetic Construct
  MOD_RES (3)..(3): Xaa is N epsilon -protected lysine
  MOD_RES (11)..(11): Xaa is N epsilon -protected lysine
  MOD_RES (18)..(18): Xaa is N epsilon -protected lysine
RFXYGGCLGNXNNYLRLXY

SEQ ID NO: 101 - protein, 19 aa, Artificial Sequence
  Synthetic Construct
  MOD_RES (10)..(10): Xaa is N epsilon -protected lysine
  MOD_RES (15)..(15): Xaa is N epsilon -protected lysine
  MOD_RES (18)..(18): Xaa is N epsilon -protected lysine
TFFYGGCRAXRNNFXRAXY

SEQ ID NO: 102 - protein, 35 aa, Artificial Sequence
  Synthetic Construct
  MOD_RES (3)..(3): Xaa is N epsilon -protected lysine
  MOD_RES (18)..(18): Xaa is N epsilon -protected lysine
NAXAGLCQTFVYGGCLAXRNNFESAEDCMRTCGGA

SEQ ID NO: 103 - protein, 24 aa, Artificial Sequence
  Synthetic Construct
  MOD_RES (7)..(7): Xaa is N epsilon -protected lysine
  MOD_RES (12)..(12): Xaa is N epsilon -protected lysine
YGGCRAXRNNFXSAEDCMRTCGGA

SEQ ID NO: 104 - protein, 22 aa, Artificial Sequence
  Synthetic Construct
  MOD_RES (14)..(14): Xaa is N epsilon -protected lysine
  MOD_RES (19)..(19): Xaa is N epsilon -protected lysine
GLCQTFVYGGCRAXRNNFXSAE

SEQ ID NO: 105 - protein, 20 aa, Artificial Sequence
  Synthetic Construct
  MOD_RES (13)..(13): Xaa is N epsilon -protected lysine
  MOD_RES (18)..(18): Xaa is N epsilon -protected lysine
LCQTFVYGGCEAXRNNFXSA

SEQ ID NO: 106 - skipped

SEQ ID NO: 107 - protein, 19 aa, Artificial Sequence
  Synthetic Construct
  MOD_RES (10)..(10): Xaa is N epsilon -protected lysine
  MOD_RES (15)..(15): Xaa is N epsilon -protected lysine
TFFYGGSRGXRNNFXTEEY

SEQ ID NO: 108 - protein, 19 aa, Artificial Sequence
  Synthetic Construct
  MOD_RES (10)..(10): Xaa is N epsilon -protected lysine
  MOD_RES (15)..(15): Xaa is N epsilon -protected lysine
RFFYGGSRGXRNNFXTEEY

SEQ ID NO: 109 - protein, 19 aa, Artificial Sequence
  Synthetic Construct
  MOD_RES (10)..(10): Xaa is N epsilon -protected lysine
  MOD_RES (15)..(15): Xaa is N epsilon -protected lysine
RFFYGGSRGXRNNFXTEEY

SEQ ID NO: 110 - protein, 19 aa, Artificial Sequence
  Synthetic Construct
  MOD_RES (10)..(10): Xaa is N epsilon -protected lysine
RFFYGGSRGXRNNFRTEEY

SEQ ID NO: 111 - protein, 19 aa, Artificial Sequence
  Synthetic Construct
  MOD_RES (10)..(10): Xaa is N epsilon -protected lysine
TFFYGGSRGXRNNFRTEEY

SEQ ID NO: 112 - protein, 19 aa, Artificial Sequence
  Synthetic Construct
TFFYGGSRGRRNNFRTEEY

SEQ ID NO: 113 - protein, 20 aa, Artificial Sequence
  Synthetic Construct
  MOD_RES (11)..(11): Xaa is N epsilon -protected lysine
  MOD_RES (16)..(16): Xaa is N epsilon -protected lysine
CTFFYGGSRGXRNNFXTEEY

SEQ ID NO: 114 - protein, 20 aa, Artificial Sequence
  Synthetic Construct
  MOD_RES (10)..(10): Xaa is N epsilon -protected lysine
  MOD_RES (15)..(15): Xaa is N epsilon -protected lysine
TFFYGGSRGXRNNFXTEEYC

SEQ ID NO: 115 - protein, 20 aa, Artificial Sequence
  Synthetic Construct
CTFFYGGSRGRRNNFRTEEY

SEQ ID NO: 116 - protein, 20 aa, Artificial Sequence
  Synthetic Construct
TFFYGGSRGRRNNFRTEEYC

SEQ ID NO: 117 - protein, 19 aa, Artificial Sequence
  Synthetic Construct
  MOD_RES (5)..(5): Xaa is N epsilon -protected lysine
  MOD_RES (10)..(10): Xaa is N epsilon -protected lysine
YEETXFNNRXGRSGGYFFT

SEQ ID NO: 118 - protein, 19 aa, Artificial Sequence
  Synthetic Construct
  MOD_RES (10)..(10): Xaa is N epsilon-acetyl-lysine
  MOD_RES (15)..(15): Xaa is N epsilon-acetyl-lysine
TFVYGGCRAXRNNFXSAED

SEQ ID NO: 119 - protein, 19 aa, Artificial Sequence
  Synthetic Construct
  MOD_RES (18)..(18): Xaa is N epsilon-acetyl-lysine
TFQYGGCMGNGNNFVTEXE

SEQ ID NO: 120 - protein, 19 aa, Artificial Sequence
  Synthetic Construct
  MOD_RES (11)..(11): Xaa is N epsilon-acetyl-lysine
SFYYGGCLGNXNNYLREEE

SEQ ID NO: 121 - protein, 19 aa, Artificial Sequence
  Synthetic Construct
  MOD_RES (10)..(10): Xaa is N epsilon-acetyl-lysine
  MOD_RES (15)..(15): Xaa is N epsilon-acetyl-lysine
  MOD_RES (18)..(18): Xaa is N epsilon-acetyl-lysine
TFFYGGCRAXRNNFXRAXY

SEQ ID NO: 122 - protein, 19 aa, Artificial Sequence
  Synthetic Construct
  MOD_RES (10)..(10): Xaa is N epsilon-acetyl-lysine
  MOD_RES (15)..(15): Xaa is N epsilon-acetyl-lysine
  MOD_RES (18)..(18): Xaa is N epsilon-acetyl-lysine
TFFYGGCRGXRNNFXRAXY

SEQ ID NO: 123 - protein, 19 aa, Artificial Sequence
  Synthetic Construct
  MOD_RES (10)..(11): Xaa is N epsilon-acetyl-lysine
  MOD_RES (15)..(15): Xaa is N epsilon-acetyl-lysine
  MOD_RES (18)..(18): Xaa is N epsilon-acetyl-lysine
TFFYGGCRAXXNNYXRAXY

SEQ ID NO: 124 - protein, 19 aa, Artificial Sequence
  Synthetic Construct
  MOD_RES (10)..(11): Xaa is N epsilon-acetyl-lysine
  MOD_RES (15)..(15): Xaa is N epsilon-acetyl-lysine
  MOD_RES (18)..(18): Xaa is N epsilon-acetyl-lysine
TFFYGGCRGXXNNFXRAXY

SEQ ID NO: 125 - protein, 19 aa, Artificial Sequence
  Synthetic Construct
  MOD_RES (10)..(10): Xaa is N epsilon-acetyl-lysine
  MOD_RES (15)..(15): Xaa is N epsilon-acetyl-lysine
  MOD_RES (18)..(18): Xaa is N epsilon-acetyl-lysine
TFQYGGCRAXRNNFXRAXY

SEQ ID NO: 126 - protein, 19 aa, Artificial Sequence
  Synthetic Construct
  MOD_RES (10)..(11): Xaa is N epsilon-acetyl-lysine
  MOD_RES (15)..(15): Xaa is N epsilon-acetyl-lysine
  MOD_RES (18)..(18): Xaa is N epsilon-acetyl-lysine
TFQYGGCRGXXNNFXRAXY

SEQ ID NO: 127 - protein, 19 aa, Artificial Sequence
  Synthetic Construct
  MOD_RES (10)..(10): Xaa is N epsilon-acetyl-lysine
  MOD_RES (15)..(15): Xaa is N epsilon-acetyl-lysine
  MOD_RES (18)..(18): Xaa is N epsilon-acetyl-lysine
TFFYGGCLGXRNNFXRAXY

SEQ ID NO: 128 - protein, 19 aa, Artificial Sequence
  Synthetic Construct
  MOD_RES (10)..(10): Xaa is N epsilon-acetyl-lysine
  MOD_RES (15)..(15): Xaa is N epsilon-acetyl-lysine
  MOD_RES (18)..(18): Xaa is N epsilon-acetyl-lysine
TFFYGGSLGXRNNFXRAXY

SEQ ID NO: 129 - protein, 19 aa, Artificial Sequence
  Synthetic Construct
  MOD_RES (10)..(11): Xaa is N epsilon-acetyl-lysine
  MOD_RES (15)..(15): Xaa is N epsilon-acetyl-lysine
  MOD_RES (18)..(18): Xaa is N epsilon-acetyl-lysine
PFFYGGCGGXXNNFXRAXY

SEQ ID NO: 130 - protein, 19 aa, Artificial Sequence
  Synthetic Construct
  MOD_RES (10)..(10): Xaa is N epsilon-acetyl-lysine
  MOD_RES (15)..(15): Xaa is N epsilon-acetyl-lysine
  MOD_RES (18)..(18): Xaa is N epsilon-acetyl-lysine
TFFYGGCRGXGNNYXRAXY

SEQ ID NO: 131 - protein, 19 aa, Artificial Sequence
  Synthetic Construct
  MOD_RES (10)..(10): Xaa is N epsilon-acetyl-lysine
  MOD_RES (18)..(18): Xaa is N epsilon-acetyl-lysine
PFFYGGCRGXRNNFLRAXY

SEQ ID NO: 132 - protein, 19 aa, Artificial Sequence
  Synthetic Construct
  MOD_RES (10)..(10): Xaa is N epsilon-acetyl-lysine
  MOD_RES (15)..(15): Xaa is N epsilon-acetyl-lysine
  MOD_RES (18)..(18): Xaa is N epsilon-acetyl-lysine
TFFYGGCRGXRNNFXREXY

SEQ ID NO: 133 - protein, 19 aa, Artificial Sequence
  Synthetic Construct
  MOD_RES (10)..(11): Xaa is n epsilon-acetyl-lysine
  MOD_RES (15)..(15): Xaa is n epsilon-acetyl-lysine
  MOD_RES (18)..(18): Xaa is n epsilon-acetyl-lysine
PFFYGGCRAXXNNFXRAXE

SEQ ID NO: 134 - protein, 19 aa, Artificial Sequence
  Synthetic Construct
  MOD_RES (10)..(10): Xaa is N epsilon-acetyl-lysine
  MOD_RES (15)..(15): Xaa is N epsilon-acetyl-lysine
  MOD_RES (18)..(18): Xaa is N epsilon-acetyl-lysine
TFFYGGCRGXRNNFXRAXD

SEQ ID NO: 135 - protein, 19 aa, Artificial Sequence
  Synthetic Construct
  MOD_RES (10)..(10): Xaa is N epsilon-acetyl-lysine
  MOD_RES (18)..(18): Xaa is N epsilon-acetyl-lysine
TFFYGGCRAXRNNFDRAXY

SEQ ID NO: 136 - protein, 19 aa, Artificial Sequence
  Synthetic Construct
  MOD_RES (10)..(11): Xaa is N epsilon-acetyl-lysine
  MOD_RES (15)..(15): Xaa is N epsilon-acetyl-lysine
TFFYGGCRGXXNNFXRAEY

SEQ ID NO: 137 - protein, 19 aa, Artificial Sequence
  Synthetic Construct
  MOD_RES (15)..(15): Xaa is N epsilon-acetyl-lysine
  MOD_RES (18)..(18): Xaa is N epsilon-acetyl-lysine
PFFYGGCGANRNNFXRAXY

SEQ ID NO: 138 - protein, 19 aa, Artificial Sequence
  Synthetic Construct
  MOD_RES (10)..(11): Xaa is N epsilon-acetyl-lysine
  MOD_RES (15)..(15): Xaa is N epsilon-acetyl-lysine
  MOD_RES (18)..(18): Xaa is N epsilon-acetyl-lysine
TFFYGGCGGXXNNFXTAXY

SEQ ID NO: 139 - protein, 19 aa, Artificial Sequence
  Synthetic Construct
  MOD_RES (18)..(18): Xaa is N epsilon-acetyl-lysine
TFFYGGCRGNRNNFLRAXY

SEQ ID NO: 140 - protein, 19 aa, Artificial Sequence
  Synthetic Construct
  MOD_RES (15)..(15): Xaa is N epsilon-acetyl-lysine
  MOD_RES (18)..(18): Xaa is N epsilon-acetyl-lysine
TFFYGGCRGNRNNFXTAXY

SEQ ID NO: 141 - protein, 19 aa, Artificial Sequence
  Synthetic Construct
  MOD_RES (15)..(15): Xaa is N epsilon-acetyl-lysine
  MOD_RES (18)..(18): Xaa is N epsilon-acetyl-lysine
TFFYGGSRGNRNNFXTAXY

SEQ ID NO: 142 - protein, 19 aa, Artificial Sequence
  Synthetic Construct
  MOD_RES (15)..(15): Xaa is N epsilon-acetyl-lysine
  MOD_RES (18)..(18): Xaa is N epsilon-acetyl-lysine
TFFYGGCLGNGNNFXRAXY

SEQ ID NO: 143 - protein, 19 aa, Artificial Sequence
  Synthetic Construct
  MOD_RES (18)..(18): Xaa is N epsilon-acetyl-lysine
TFFYGGCLGNRNNFLRAXY

SEQ ID NO: 144 - protein, 19 aa, Artificial Sequence
  Synthetic Construct
  MOD_RES (15)..(15): Xaa is N epsilon-acetyl-lysine
  MOD_RES (18)..(18): Xaa is N epsilon-acetyl-lysine
TFFYGGCLGNRNNFXTAXY

SEQ ID NO: 145 - protein, 19 aa, Artificial Sequence
  Synthetic Construct
  MOD_RES (15)..(15): Xaa is N epsilon-acetyl-lysine
  MOD_RES (18)..(18): Xaa is N epsilon-acetyl-lysine
TFFYGGCRGNGNNFXSAXY

SEQ ID NO: 146 - protein, 19 aa, Artificial Sequence
  Synthetic Construct
  MOD_RES (10)..(11): Xaa is n epsilon-acetyl-lysine
  MOD_RES (18)..(18): Xaa is n epsilon-acetyl-lysine
TFFYGGCRGXXNNFDREXY

SEQ ID NO: 147 - protein, 19 aa, Artificial Sequence
  Synthetic Construct
  MOD_RES (10)..(10): Xaa is N epsilon-acetyl-lysine
  MOD_RES (18)..(18): Xaa is N epsilon-acetyl-lysine
TFFYGGCRGXRNNFLREXE

SEQ ID NO: 148 - protein, 19 aa, Artificial Sequence
  Synthetic Construct
  MOD_RES (10)..(10): Xaa is N epsilon-acetyl-lysine
  MOD_RES (18)..(18): Xaa is N epsilon-acetyl-lysine
TFFYGGCRGXGNNFDRAXY

SEQ ID NO: 149 - protein, 19 aa, Artificial Sequence
  Synthetic Construct
  MOD_RES (10)..(10): Xaa is N epsilon-acetyl-lysine
  MOD_RES (18)..(18): Xaa is N epsilon-acetyl-lysine
TFFYGGSRGXGNNFDRAXY

SEQ ID NO: 150 - protein, 19 aa, Artificial Sequence
  Synthetic Construct
  MOD_RES (18)..(18): Xaa is N epsilon-acetyl-lysine
TFFYGGCRGNGNNFVTAXY

SEQ ID NO: 151 - protein, 19 aa, Artificial Sequence
  Synthetic Construct
  MOD_RES (10)..(10): Xaa is N epsilon-acetyl-lysine
  MOD_RES (18)..(18): Xaa is N epsilon-acetyl-lysine
PFFYGGCGGXGNNYVTAXY

SEQ ID NO: 152 - protein, 19 aa, Artificial Sequence
  Synthetic Construct
  MOD_RES (10)..(10): Xaa is N epsilon-acetyl-lysine
  MOD_RES (18)..(18): Xaa is N epsilon-acetyl-lysine
TFFYGGCLGXGNNFLTAXY

SEQ ID NO: 153 - protein, 19 aa, Artificial Sequence
  Synthetic Construct
  MOD_RES (11)..(11): Xaa is N epsilon-acetyl-lysine
  MOD_RES (18)..(18): Xaa is N epsilon-acetyl-lysine
SFFYGGCLGNXNNFLTAXY

SEQ ID NO: 154 - protein, 19 aa, Artificial Sequence
  Synthetic Construct
  MOD_RES (11)..(11): Xaa is N epsilon-acetyl-lysine
  MOD_RES (18)..(18): Xaa is N epsilon-acetyl-lysine
TFFYGGCGGNXNNFVREXY

SEQ ID NO: 155 - protein, 19 aa, Artificial Sequence
  Synthetic Construct
  MOD_RES (11)..(11): Xaa is N epsilon-acetyl-lysine
  MOD_RES (18)..(18): Xaa is N epsilon-acetyl-lysine
TFFYGGCMGNXNNFVREXY

SEQ ID NO: 156 - protein, 19 aa, Artificial Sequence
  Synthetic Construct
  MOD_RES (11)..(11): Xaa is N epsilon-acetyl-lysine
  MOD_RES (18)..(18): Xaa is N epsilon-acetyl-lysine
TFFYGGSMGNXNNFVREXY

SEQ ID NO: 157 - protein, 19 aa, Artificial Sequence
  Synthetic Construct
  MOD_RES (18)..(18): Xaa is N epsilon-acetyl-lysine
PFFYGGCLGNRNNYVREXY

SEQ ID NO: 158 - protein, 19 aa, Artificial Sequence
  Synthetic Construct
  MOD_RES (18)..(18): Xaa is N epsilon-acetyl-lysine
TFFYGGCLGNRNNFVREXY

SEQ ID NO: 159 - protein, 19 aa, Artificial Sequence
  Synthetic Construct
  MOD_RES (11)..(11): Xaa is N epsilon-acetyl-lysine
  MOD_RES (18)..(18): Xaa is N epsilon-acetyl-lysine
TFFYGGCLGNXNNYVREXY

SEQ ID NO: 160 - protein, 19 aa, Artificial Sequence
  Synthetic Construct
  MOD_RES (18)..(18): Xaa is N epsilon-acetyl-lysine
TFFYGGCGGNGNNFLTAXY

SEQ ID NO: 161 - protein, 19 aa, Artificial Sequence
  Synthetic Construct
  MOD_RES (15)..(15): Xaa is N epsilon-acetyl-lysine
TFFYGGCRGNGNNFXSAEY

SEQ ID NO: 162 - protein, 19 aa, Artificial Sequence
  Synthetic Construct
  MOD_RES (11)..(11): Xaa is N epsilon-acetyl-lysine
  MOD_RES (15)..(15): Xaa is N epsilon-acetyl-lysine
PFFYGGCLGNXNNFXTAEY

SEQ ID NO: 163 - protein, 19 aa, Artificial Sequence
  Synthetic Construct
  MOD_RES (15)..(15): Xaa is N epsilon-acetyl-lysine
TFFYGGCRGNRNNFXTEEY

SEQ ID NO: 164 - protein, 19 aa, Artificial Sequence
  Synthetic Construct
  MOD_RES (10)..(10): Xaa is N epsilon-acetyl-lysine
  MOD_RES (15)..(15): Xaa is N epsilon-acetyl-lysine
TFFYGGCRGXRNNFXTEED

SEQ ID NO: 165 - protein, 19 aa, Artificial Sequence
  Synthetic Construct
  MOD_RES (18)..(18): Xaa is N epsilon-acetyl-lysine
PFFYGGCGGNGNNFVREXY

SEQ ID NO: 166 - protein, 19 aa, Artificial Sequence
  Synthetic Construct
  MOD_RES (18)..(18): Xaa is N epsilon-acetyl-lysine
SFFYGGCMGNGNNFVREXY

SEQ ID NO: 167 - protein, 19 aa, Artificial Sequence
  Synthetic Construct
  MOD_RES (18)..(18): Xaa is N epsilon-acetyl-lysine
PFFYGGCGGNGNNFLREXY

SEQ ID NO: 168 - protein, 19 aa, Artificial Sequence
  Synthetic Construct
  MOD_RES (18)..(18): Xaa is N epsilon-acetyl-lysine
TFFYGGCLGNGNNFVREXY

SEQ ID NO: 169 - protein, 19 aa, Artificial Sequence
  Synthetic Construct
  MOD_RES (18)..(18): Xaa is N epsilon-acetyl-lysine
SFFYGGCLGNGNNYLREXY

SEQ ID NO: 170 - protein, 19 aa, Artificial Sequence
  Synthetic Construct
  MOD_RES (18)..(18): Xaa is N epsilon-acetyl-lysine
TFFYGGSLGNGNNFVREXY

SEQ ID NO: 171 - protein, 19 aa, Artificial Sequence
  Synthetic Construct
  MOD_RES (10)..(10): Xaa is N epsilon-acetyl-lysine
TFFYGGCLGXGNNFVSAEY

SEQ ID NO: 172 - protein, 19 aa, Artificial Sequence
  Synthetic Construct
  MOD_RES (11)..(11): Xaa is n epsilon-acetyl-lysine
TFFYGGCLGNXNNYLREEY

SEQ ID NO: 173 - protein, 21 aa, Artificial Sequence
  Synthetic Construct
  MOD_RES (11)..(11): Xaa is N epsilon-acetyl-lysine
ARIIRYFYNAXAGLCQTFVYG

SEQ ID NO: 174 - protein, 22 aa, Artificial Sequence
  Synthetic Construct
  MOD_RES (7)..(7): Xaa is N epsilon-acetyl-lysine
  MOD_RES (12)..(12): Xaa is N epsilon-acetyl-lysine
YGGCRAXRNNYXSAEDCMRTCG

SEQ ID NO: 175 - protein, 19 aa, Artificial Sequence
  Synthetic Construct
  MOD_RES (10)..(10): Xaa is N epsilon-acetyl-lysine
  MOD_RES (15)..(15): Xaa is N epsilon-acetyl-lysine
TFFYGGCRGXRNNFXTEEY

SEQ ID NO: 176 - protein, 19 aa, Artificial Sequence
  Synthetic Construct
  MOD_RES (1)..(1): Xaa is N epsilon-acetyl-lysine
  MOD_RES (10)..(10): Xaa is N epsilon-acetyl-lysine
  MOD_RES (15)..(15): Xaa is N epsilon-acetyl-lysine
XFFYGGCRGXRNNFXTEEY

SEQ ID NO: 177 - protein, 19 aa, Artificial Sequence
  Synthetic Construct
  MOD_RES (10)..(10): Xaa is N epsilon-acetyl-lysine
  MOD_RES (15)..(15): Xaa is N epsilon-acetyl-lysine
TFYYGGCRGXRNNYXTEEY

SEQ ID NO: 178 - protein, 20 aa, Artificial Sequence
  Synthetic Construct
  MOD_RES (11)..(11): Xaa is N epsilon-acetyl-lysine
  MOD_RES (16)..(16): Xaa is N epsilon-acetyl-lysine
CTFFYGCCRGXRNNFXTEEY

SEQ ID NO: 179 - protein, 20 aa, Artificial Sequence
  Synthetic Construct
  MOD_RES (10)..(10): Xaa is N epsilon-acetyl-lysine
  MOD_RES (15)..(15): Xaa is N epsilon-acetyl-lysine
TFFYGGCRGXRNNFXTEEYC

SEQ ID NO: 180 - protein, 20 aa, Artificial Sequence
  Synthetic Construct
  MOD_RES (11)..(11): Xaa is N epsilon-acetyl-lysine
  MOD_RES (16)..(16): Xaa is N epsilon-acetyl-lysine
CTFFYGSCRGXRNNFXTEEY

SEQ ID NO: 181 - protein, 19 aa, Artificial Sequence
  Synthetic Construct
  MOD_RES (10)..(10): Xaa is N epsilon-acetyl-lysine
  MOD_RES (15)..(15): Xaa is N epsilon-acetyl-lysine
PFFYGGCRGXRNNFXTEEY

SEQ ID NO: 182 - protein, 19 aa, Artificial Sequence
  Synthetic Construct
  MOD_RES (10)..(10): Xaa is N epsilon-acetyl-lysine
  MOD_RES (15)..(15): Xaa is N epsilon-acetyl-lysine
  MOD_RES (17)..(17): Xaa is N epsilon-acetyl-lysine
TFFYGGCRGXRNNFXTXEY

SEQ ID NO: 183 - protein, 19 aa, Artificial Sequence
  Synthetic Construct
  MOD_RES (7)..(7): Xaa is N epsilon-acetyl-lysine
  MOD_RES (10)..(10): Xaa is N epsilon-acetyl-lysine
  MOD_RES (15)..(15): Xaa is N epsilon-acetyl-lysine
TFFYGGXRGXRNNFXTEEY

SEQ ID NO: 184 - protein, 19 aa, Artificial Sequence
  Synthetic Construct
  MOD_RES (10)..(10): Xaa is N epsilon-acetyl-lysine
  MOD_RES (15)..(15): Xaa is N epsilon-acetyl-lysine
  MOD_RES (17)..(17): Xaa is N epsilon-acetyl-lysine
TFFYGGCRGXRNNFXTXRY

SEQ ID NO: 185 - protein, 19 aa, Artificial Sequence
  Synthetic Construct
  MOD_RES (7)..(7): Xaa is N epsilon-acetyl-lysine
  MOD_RES (10)..(10): Xaa is N epsilon-acetyl-lysine
  MOD_RES (15)..(15): Xaa is N epsilon-acetyl-lysine
TFFYGGXRGXRNNFXTAEY

SEQ ID NO: 186 - protein, 19 aa, Artificial Sequence
  Synthetic Construct
  MOD_RES (7)..(7): Xaa is N epsilon-acetyl-lysine
  MOD_RES (10)..(10): Xaa is N epsilon-acetyl-lysine
  MOD_RES (15)..(15): Xaa is N epsilon-acetyl-lysine
TFFYGGXRGXRNNFXTAGY

SEQ ID NO: 187 - protein, 19 aa, Artificial Sequence
  Synthetic Construct
  MOD_RES (7)..(7): Xaa is N epsilon-acetyl-lysine
  MOD_RES (10)..(10): Xaa is N epsilon-acetyl-lysine
  MOD_RES (15)..(15): Xaa is N epsilon-acetyl-lysine
  MOD_RES (18)..(18): Xaa is N epsilon-acetyl-lysine
TFFYGGXRGXRNNFXREXY

SEQ ID NO: 188 - protein, 19 aa, Artificial Sequence
  Synthetic Construct
  MOD_RES (7)..(7): Xaa is N epsilon-acetyl-lysine
  MOD_RES (10)..(10): Xaa is N epsilon-acetyl-lysine
  MOD_RES (15)..(15): Xaa is N epsilon-acetyl-lysine
  MOD_RES (18)..(18): Xaa is N epsilon-acetyl-lysine
TFFYGGXRGXRNNFXRAXY

SEQ ID NO: 189 - protein, 19 aa, Artificial Sequence
  Synthetic Construct
  MOD_RES (15)..(15): Xaa is N epsilon-acetyl-lysine
TFFYGGCLGNRNNFXTEEY

SEQ ID NO: 190 - protein, 19 aa, Artificial Sequence
  Synthetic Construct
  MOD_RES (10)..(10): Xaa is N epsilon-acetyl-lysine
  MOD_RES (15)..(15): Xaa is N epsilon-acetyl-lysine
TFFYGCGRGXRNNFXTEEY

SEQ ID NO: 191 - protein, 19 aa, Artificial Sequence
  Synthetic Construct
  MOD_RES (10)..(10): Xaa is N epsilon-acetyl-lysine
  MOD_RES (15)..(15): Xaa is N epsilon-acetyl-lysine
TFFYGGRCGXRNNFXTEEY

SEQ ID NO: 192 - protein, 19 aa, Artificial Sequence
  Synthetic Construct
  MOD_RES (10)..(10): Xaa is N epsilon-acetyl-lysine
  MOD_RES (15)..(15): Xaa is N epsilon-acetyl-lysine
TFQYGGCRGXRNNFXTEEY

SEQ ID NO: 193 - protein, 19 aa, Artificial Sequence
  Synthetic Construct
  MOD_RES (3)..(3): Xaa is N epsilon-acetyl-lysine
  MOD_RES (11)..(11): Xaa is N epsilon-acetyl-lysine
YNXEFGTFNTXGCERGYRF

SEQ ID NO: 194 - protein, 19 aa, Artificial Sequence
  Synthetic Construct
  MOD_RES (3)..(3): Xaa is N epsilon-acetyl-lysine
RFXYGGCLGNMNNFETLEE

SEQ ID NO: 195 - protein, 19 aa, Artificial Sequence
  Synthetic Construct
  MOD_RES (3)..(3): Xaa is N epsilon-acetyl-lysine
  MOD_RES (11)..(11): Xaa is N epsilon-acetyl-lysine
  MOD_RES (18)..(18): Xaa is N epsilon-acetyl-lysine
RFXYGGCLGNXNNFLRLXY

SEQ ID NO: 196 - protein, 19 aa, Artificial Sequence
  Synthetic Construct
  MOD_RES (3)..(3): Xaa is N epsilon-acetyl-lysine
  MOD_RES (11)..(11): Xaa is N epsilon-acetyl-lysine
  MOD_RES (18)..(18): Xaa is N epsilon-acetyl-lysine
RFXYGGCLGNXNNYLRLXY

SEQ ID NO: 197 - protein, 22 aa, Artificial Sequence
  Synthetic Construct
  MOD_RES (1)..(1): Xaa is N epsilon-acetyl-lysine
  MOD_RES (3)..(3): Xaa is N epsilon-acetyl-lysine
  MOD_RES (5)..(5): Xaa is N epsilon-acetyl-lysine
  MOD_RES (7)..(8): Xaa is N epsilon-acetyl-lysine
  MOD_RES (12)..(12): Xaa is N epsilon-acetyl-lysine
  MOD_RES (20)..(20): Xaa is N epsilon-acetyl-lysine
XTXRXRXXQRVXIAYEEIFXNY

SEQ ID NO: 198 - protein, 15 aa, Artificial Sequence
  Synthetic Construct
  MOD_RES (1)..(1): Xaa is N epsilon-acetyl-lysine
  MOD_RES (3)..(3): Xaa is N epsilon-acetyl-lysine
  MOD_RES (5)..(5): Xaa is N epsilon-acetyl-lysine
  MOD_RES (7)..(8): Xaa is N epsilon-acetyl-lysine
  MOD_RES (12)..(12): Xaa is N epsilon-acetyl-lysine
XTXRXRXXQRVXIAY

SEQ ID NO: 199 - protein, 16 aa, Artificial Sequence
  Synthetic Construct
  MOD_RES (4)..(4): Xaa is N epsilon-acetyl-lysine
  MOD_RES (13)..(13): Xaa is N epsilon-acetyl-lysine
  MOD_RES (15)..(16): Xaa is N epsilon-acetyl-lysine
RQIXIWFQNRRMXWXX

SEQ ID NO: 200 - protein, 19 aa, Artificial Sequence
  Synthetic Construct
  MOD_RES (10)..(10): Xaa is N epsilon-acetyl-lysine
  MOD_RES (15)..(15): Xaa is N epsilon-acetyl-lysine
TFFYGGSRGXRNNFXTEEY

SEQ ID NO: 201 - protein, 59 aa, Artificial Sequence; identical to SEQ ID NO: 98
  Synthetic Construct
  MOD_RES (27)..(27): Xaa is N epsilon-acetyl-lysine
  MOD_RES (42)..(42): Xaa is N epsilon-acetyl-lysine
  MOD_RES (47)..(47): Xaa is N epsilon-acetyl-lysine

SEQ ID NO: 202 - protein, 19 aa, Artificial Sequence
  Synthetic Construct
  MOD_RES (10)..(10): Xaa is N epsilon-acetyl-lysine
  MOD_RES (15)..(15): Xaa is N epsilon-acetyl-lysine
  MOD_RES (17)..(17): Xaa is N epsilon-acetyl-lysine
TFFYGGCRGXRNNFXTXEY

SEQ ID NO: 203 - protein, 19 aa, Artificial Sequence
  Synthetic Construct
  MOD_RES (3)..(3): Xaa is N epsilon-acetyl-lysine
  MOD_RES (11)..(11): Xaa is N epsilon-acetyl-lysine
  MOD_RES (18)..(18): Xaa is N epsilon-acetyl-lysine
RFXYGGCLGNXNNYLRLXY

SEQ ID NO: 204 - protein, 19 aa, Artificial Sequence
  Synthetic Construct
  MOD_RES (10)..(10): Xaa is N epsilon-acetyl-lysine
  MOD_RES (15)..(15): Xaa is N epsilon-acetyl-lysine
  MOD_RES (18)..(18): Xaa is N epsilon-acetyl-lysine
TFFYGGCRAXRNNFXRAXY

SEQ ID NO: 205 - protein, 35 aa, Artificial Sequence; identical to SEQ ID NO: 102
  Synthetic Construct
  MOD_RES (3)..(3): Xaa is N epsilon-acetyl-lysine
  MOD_RES (18)..(18): Xaa is N epsilon-acetyl-lysine

SEQ ID NO: 206 - protein, 24 aa, Artificial Sequence
  Synthetic Construct
  MOD_RES (7)..(7): Xaa is N epsilon-acetyl-lysine
  MOD_RES (12)..(12): Xaa is N epsilon-acetyl-lysine
YGGCRAXRNNFXSAEDCMRTCGGA

SEQ ID NO: 207 - protein, 22 aa, Artificial Sequence
  Synthetic Construct
  MOD_RES (14)..(14): Xaa is N epsilon-acetyl-lysine
  MOD_RES (19)..(19): Xaa is N epsilon-acetyl-lysine
GLCQTFVYGGCRAXRNNFXSAE

SEQ ID NO: 208 - protein, 20 aa, Artificial Sequence
  Synthetic Construct
  MOD_RES (13)..(13): Xaa is N epsilon-acetyl-lysine
  MOD_RES (18)..(18): Xaa is N epsilon-acetyl-lysine
LCQTFVYGGCEAXRNNFXSA

SEQ ID NO: 209 - protein, 19 aa, Artificial Sequence
  Synthetic Construct
  MOD_RES (10)..(10): Xaa is N epsilon-acetyl-lysine
  MOD_RES (15)..(15): Xaa is N epsilon-acetyl-lysine
TFFYGGSRGXRNNFXTEEY

SEQ ID NO: 210 - protein, 19 aa, Artificial Sequence
  Synthetic Construct
  MOD_RES (10)..(10): Xaa is N epsilon-acetyl-lysine
  MOD_RES (15)..(15): Xaa is N epsilon-acetyl-lysine
RFFYGGSRGXRNNFXTEEY

SEQ ID NO: 211 - protein, 19 aa, Artificial Sequence
  Synthetic Construct
  MOD_RES (10)..(10): Xaa is N epsilon-acetyl-lysine
  MOD_RES (15)..(15): Xaa is N epsilon-acetyl-lysine
RFFYGGSRGXRNNFXTEEY

SEQ ID NO: 212 - protein, 19 aa, Artificial Sequence
  Synthetic Construct
  MOD_RES (10)..(10): Xaa is N epsilon-acetyl-lysine
RFFYGGSRGXRNNFRTEEY

SEQ ID NO: 213 - protein, 19 aa, Artificial Sequence
  Synthetic Construct
  MOD_RES (10)..(10): Xaa is N epsilon-acetyl-lysine
TFFYGGSRGXRNNFRTEEY

SEQ ID NO: 214 - protein, 20 aa, Artificial Sequence
  Synthetic Construct
  MOD_RES (11)..(11): Xaa is N epsilon-acetyl-lysine
  MOD_RES (16)..(16): Xaa is N epsilon-acetyl-lysine
CTFFYGGSRGXRNNFXTEEY

SEQ ID NO: 215 - protein, 20 aa, Artificial Sequence
  Synthetic Construct
  MOD_RES (10)..(10): Xaa is N epsilon-acetyl-lysine
  MOD_RES (15)..(15): Xaa is N epsilon-acetyl-lysine
TFFYGGSRGXRNNFXTEEYC

SEQ ID NO: 216 - protein, 19 aa, Artificial Sequence
  Synthetic Construct
  MOD_RES (5)..(5): Xaa is N epsilon-acetyl-lysine
  MOD_RES (10)..(10): Xaa is N epsilon-acetyl-lysine
YEETXFNNRXGRSGGYFFT

SEQ ID NO: 217 - protein, 19 aa, Artificial Sequence
  Synthetic Construct
  MOD_RES (1)..(19): Xaa is Ala, Arg, Asn, Asp, Cys, Gln, Glu, Gly, His, Ile, Leu, Lys, Met, Phe, Pro, Ser, Thr, Trp, Tyr, Val or absent
XXXXXXXXXXXXXXXXXXX

SEQ ID NO: 218 - protein, 6 aa, Artificial Sequence
  Synthetic Construct
  MOD_RES (1)..(1): Xaa is N epsilon -protected lysine
  MOD_RES (3)..(3): Xaa is Asn or Gln
  MOD_RES (4)..(4): Xaa is Asn or Gln
  MOD_RES (5)..(5): Xaa is Phe, Tyr,or Trp
  MOD_RES (6)..(6): Xaa is N epsilon -protected lysine
XRXXXX

SEQ ID NO: 219 - protein, 21 aa, Artificial Sequence
  Synthetic Construct
  MOD_RES (1)..(1): Xaa is Cys, Gly, Arg, Ser, Tyr, Phe, Thr, or absent
  MOD_RES (2)..(2): Xaa is Gly, Arg, Ser, Tyr, Phe, Thr, or absent
  MOD_RES (3)..(3): Xaa is Gly, Arg, Ser, Tyr, Phe, or absent
  MOD_RES (4)..(4): Xaa is Gly, Arg, Ser, Tyr, Phe, or absent
  MOD_RES (5)..(5): Xaa is Gly, Arg, Ser, Tyr, or absent
  MOD_RES (6)..(6): Xaa is Gly, Arg, Ser, or absent
  MOD_RES (7)..(7): Xaa is Gly, Arg, Ser, or absent
  MOD_RES (8)..(8): Xaa is Gly, Arg, Ser, or absent
  MOD_RES (9)..(9): Xaa is Gly, Arg, or absent
  MOD_RES (10)..(10): Xaa is Gly or absent
  MOD_RES (11)..(11): Xaa is N epsilon -protected lysine
  MOD_RES (13)..(13): Xaa is Asn or Gln
  MOD_RES (14)..(14): Xaa is Asn or Gln
  MOD_RES (15)..(15): Xaa is Phe, Tyr, or Trp
  MOD_RES (16)..(16): Xaa is N epsilon -protected lysine
  MOD_RES (17)..(17): Xaa is Cys, Tyr, Thr, or absent
  MOD_RES (18)..(18): Xaa is Cys, Tyr, Glu, or absent
  MOD_RES (19)..(19): Xaa is Glu or absent
  MOD_RES (20)..(20): Xaa is Tyr or absent
  MOD_RES (21)..(21): Xaa is Cys or absent
XXXXXXXXXXXRXXXXXXXXX

SEQ ID NO: 220 - protein, 6 aa, Artificial Sequence
  Synthetic Construct
  MOD_RES (1)..(1): Xaa is N epsilon -protected lysine or D- N epsilon -protected lysine
  MOD_RES (2)..(2): Xaa is Arg or D-Arg
  MOD_RES (5)..(5): Xaa is Phe or D-Phe
  MOD_RES (6)..(6): Xaa is N epsilon -protected lysine or D-n epsilon -protected lysine
XXNNXX

SEQ ID NO: 221 - protein, 7 aa, Artificial Sequence
  Synthetic Construct
  MOD_RES (1)..(1): Xaa is N epsilon -protected lysine or D-n epsilon -protected lysine
  MOD_RES (2)..(2): Xaa is Arg or D-Arg
  MOD_RES (5)..(5): Xaa is Phe or D-Phe
  MOD_RES (6)..(6): Xaa is N epsilon -protected lysine or D-n epsilon -protected lysine
  MOD_RES (7)..(7): Xaa is Tyr or D-Tyr
XXNNXXX

SEQ ID NO: 222 - protein, 22 aa, Artificial Sequence
  Synthetic Construct
  MOD_RES (1)..(1): Xaa is Cys or absent
  MOD_RES (2)..(2): Xaa is Cys, Thr, or absent
  MOD_RES (3)..(3): Xaa is Cys, Phe, or absent
  MOD_RES (4)..(4): Xaa is Cys, Phe, or absent
  MOD_RES (5)..(5): Xaa is Cys, Tyr, or absent
  MOD_RES (6)..(6): Xaa is Cys, Gly, or absent
  MOD_RES (7)..(7): Xaa is Cys, Gly, or absent
  MOD_RES (8)..(8): Xaa is Cys, Ser, or absent
  MOD_RES (9)..(9): Xaa is Cys, Arg, or absent
  MOD_RES (10)..(10): Xaa is Cys, Gly, or absent
  MOD_RES (11)..(11): Xaa is N epsilon -protected lysine or D-N epsilon -protected lysine
  misc_feature (12)..(12): Xaa can be any naturally occurring amino acid
  MOD_RES (15)..(15): Xaa is Phe or D-Phe
  MOD_RES (16)..(16): Xaa is N epsilon -protected lysine or D-N epsilon -protected lysine
  MOD_RES (17)..(17): Xaa is Tyr or D-Tyr
  MOD_RES (18)..(18): Xaa is Cys, Tyr, Thr, or absent
  MOD_RES (19)..(19): Xaa is Cys, Tyr, Glu, or absent
  MOD_RES (20)..(20): Xaa is Glu or absent
  MOD_RES (21)..(21): Xaa is Tyr or absent
  MOD_RES (22)..(22): Xaa is Cys or absent
XXXXXXXXXXXXNNXXXXXXXX

SEQ ID NO: 223 - protein, 6 aa, Artificial Sequence
  Synthetic Construct
  MOD_RES (1)..(1): Xaa is N epsilon-acetyl-lysine
  MOD_RES (3)..(3): Xaa is Asn or Gln
  MOD_RES (4)..(4): Xaa is Asn or Gln
  MOD_RES (5)..(5): Xaa is Phe, Tyr, or Trp
  MOD_RES (6)..(6): Xaa is N epsilon-acetyl-lysine
XRXXXX

SEQ ID NO: 224 - protein, 21 aa, Artificial Sequence
  Synthetic Construct
  MOD_RES (1)..(1): Xaa is Cys or absent
  MOD_RES (2)..(2): Xaa is Cys, Thr, or absent
  MOD_RES (3)..(3): Xaa is Cys, Phe, or absent
  MOD_RES (4)..(4): Xaa is Cys, Phe, or absent
  MOD_RES (5)..(5): Xaa is Cys, Tyr, or absent
  MOD_RES (6)..(6): Xaa is Cys, Gly, or absent
  MOD_RES (7)..(7): Xaa is Cys, Gly, or absent
  MOD_RES (8)..(8): Xaa is Cys, Ser, or absent
  MOD_RES (9)..(9): Xaa is Cys, Arg, or absent
  MOD_RES (10)..(10): Xaa is Cys, Gly, or absent
  MOD_RES (11)..(11): Xaa is N epsilon-acetyl-lysine
  MOD_RES (13)..(13): Xaa is Asn or Gln
  MOD_RES (14)..(14): Xaa is Asn or Gln
  MOD_RES (15)..(15): Xaa is Phe, Tyr, or Trp
  MOD_RES (16)..(16): Xaa is N epsilon-acetyl-lysine
  MOD_RES (17)..(17): Xaa is Cys, Tyr, Thr, or absent
  MOD_RES (18)..(18): Xaa is Cys, Tyr, Glu, or absent
  MOD_RES (19)..(19): Xaa is Glu or absent
  MOD_RES (20)..(20): Xaa is Tyr or absent
  MOD_RES (21)..(21): Xaa is Cys or absent
XXXXXXXXXXXRXXXXXXXXX

SEQ ID NO: 225 - protein, 6 aa, Artificial Sequence
  Synthetic Construct
  MOD_RES (1)..(1): Xaa is N epsilon-acetyl-lysine
  MOD_RES (6)..(6): Xaa is N epsilon-acetyl-lysine
XRNNFX

SEQ ID NO: 226 - protein, 7 aa, Artificial Sequence
  Synthetic Construct
  MOD_RES (1)..(1): Xaa is N epsilon-acetyl-lysine
  MOD_RES (6)..(6): Xaa is N epsilon-acetyl-lysine
XRNNFXY

SEQ ID NO: 227 - protein, 8 aa, Artificial Sequence
  Synthetic Construct
  MOD_RES (1)..(1): Xaa is n epsilon-acetyl-lysine
  MOD_RES (6)..(6): Xaa is n epsilon-acetyl-lysine
XRNNFXYC

SEQ ID NO: 228 - protein, 6 aa, Artificial Sequence
  Synthetic Construct
  MOD_RES (1)..(1): Xaa is N epsilon-acetyl-lysine or D-N epsilon-acetyl-lysine
  MOD_RES (2)..(2): Xaa is Arg or D-Arg
  MOD_RES (5)..(5): Xaa is Phe or D-Phe
  MOD_RES (6)..(6): Xaa is N epsilon-acetyl-lysine or D-N epsilon-acetyl-lysine
XXNNXX

SEQ ID NO: 229 - protein, 7 aa, Artificial Sequence
  Synthetic Construct
  MOD_RES (1)..(1): Xaa is N epsilon-acetyl-lysine or D-N epsilon-acetyl-lysine
  MOD_RES (2)..(2): Xaa is Arg or D-Arg
  MOD_RES (5)..(5): Xaa is Phe or D-Phe
  MOD_RES (6)..(6): Xaa is N epsilon-acetyl-lysine or D-N epsilon-acetyl-lysine
  MOD_RES (7)..(7): Xaa is Tyr or D-Tyr
XXNNXXX

SEQ ID NO: 230 - protein, 22 aa, Artificial Sequence
  Synthetic Construct
  MOD_RES (1)..(1): Xaa is Cys or absent
  MOD_RES (2)..(2): Xaa is Cys, Thr, or absent
  MOD_RES (3)..(3): Xaa is Cys, Phe, or absent
  MOD_RES (4)..(4): Xaa is Cys, Phe, or absent
  MOD_RES (5)..(5): Xaa is Cys, Tyr, or absent
  MOD_RES (6)..(6): Xaa is Cys, Gly, or absent
  MOD_RES (7)..(7): Xaa is Cys, Gly, or absent
  MOD_RES (8)..(8): Xaa is Cys, Ser, or absent
  MOD_RES (9)..(9): Xaa is Cys, Arg, or absent
  MOD_RES (10)..(10): Xaa is Cys, Gly, or absent
  MOD_RES (11)..(11): Xaa is N epsilon-acetyl-lysine or D-N epsilon acetyl-lysine
  MOD_RES (12)..(12): Xaa is Arg or D-Arg
  MOD_RES (15)..(15): Xaa is Phe or D-Phe
  MOD_RES (16)..(16): Xaa is N epsilon-acetyl-lysine or D-N epsilon-acetyl-lysine
  MOD_RES (17)..(17): Xaa is Tyr or D-Tyr
  MOD_RES (18)..(18): Xaa is Cys, Tyr, Thr, or absent
  MOD_RES (19)..(19): Xaa is Cys, Tyr, Glu, or absent
  MOD_RES (20)..(20): Xaa is Glu or absent
  MOD_RES (21)..(21): Xaa is Tyr or absent
  MOD_RES (22)..(22): Xaa is Cys or absent
XXXXXXXXXXXXNNXXXXXXXX

SEQ ID NO: 231 - protein, 19 aa, Artificial Sequence
  Synthetic Construct
  MOD_RES (8)..(8): Xaa is D-Arg
  MOD_RES (10)..(10): Xaa is D-N epsilon-acetyl-lysine
  MOD_RES (11)..(11): Xaa is D-Arg
  MOD_RES (15)..(15): Xaa is D-N epsilon-acetyl-lysine
TFFYGGSXGXXNNFXTEEY

SEQ ID NO: 232 - protein, 19 aa, Artificial Sequence
  Synthetic Construct
  MOD_RES (8)..(8): Xaa is D-Arg
  MOD_RES (10)..(10): Xaa is D-N epsilon-acetyl-lysine
  MOD_RES (11)..(11): Xaa is D-Arg
  MOD_RES (15)..(15): Xaa is N epsilon-acetyl-lysine
TFFYGGSXGXXNNFXTEEY

SEQ ID NO: 233 - protein, 18 aa, Artificial Sequence
  Synthetic Construct
  MOD_RES (8)..(8): Xaa is N epsilon-acetyl-lysine
  MOD_RES (13)..(13): Xaa is N epsilon-acetyl-lysine
FYGGSRGXRNNFXTEEYC

SEQ ID NO: 234 - protein, 18 aa, Artificial Sequence
  Synthetic Construct
  MOD_RES (8)..(8): Xaa is D-N epsilon-acetyl-lysine
  MOD_RES (9)..(9): Xaa is D-Arg
  MOD_RES (12)..(12): Xaa is D-Phe
  MOD_RES (13)..(13): Xaa is N epsilon-acetyl-lysine
FYGGSRGXXNNXXTEEYC

SEQ ID NO: 235 - protein, 18 aa, Artificial Sequence
  Synthetic Construct
  MOD_RES (8)..(8): Xaa is D-N-epsilon-acetyl-lysine
  MOD_RES (9)..(9): Xaa is D-Arg
  MOD_RES (12)..(12): Xaa is D-Phe
  MOD_RES (13)..(13): Xaa is D-N-epsilon-acetyl-lysine
FYGGSRGXXNNXXTEEYC

SEQ ID NO: 236 - protein, 18 aa, Artificial Sequence
  Synthetic Construct
  MOD_RES (8)..(8): Xaa is D-N epsilon-acetyl-lysine
  MOD_RES (9)..(9): Xaa is D-Arg
  MOD_RES (12)..(12): Xaa is D-Phe
  MOD_RES (13)..(13): Xaa is D-N epsilon-acetyl-lysine
  MOD_RES (17)..(17): Xaa is D-Tyr
FYGGSRGXXNNXXTEEXC

SEQ ID NO: 237 - protein, 18 aa, Artificial Sequence
  Synthetic Construct
  MOD_RES (1)..(1): Xaa is D-Phe
  MOD_RES (2)..(2): Xaa is D-Tyr
  MOD_RES (6)..(6): Xaa is D-Arg
  MOD_RES (8)..(8): Xaa is D-N epsilon-acetyl-lysine
  MOD_RES (9)..(9): Xaa is D-Arg
  MOD_RES (12)..(12): Xaa is D-Phe
  MOD_RES (13)..(13): Xaa is D-N epsilon-acetyl-lysine
  MOD_RES (16)..(16): Xaa is D-Glu
  MOD_RES (17)..(17): Xaa is D-Tyr
XXGGSXGXXNNXXTEXXC

SEQ ID NO: 238 - protein, 16 aa, Artificial Sequence
  Synthetic Construct
  MOD_RES (6)..(6): Xaa is N epsilon-acetyl-lysine
  MOD_RES (11)..(11): Xaa is N epsilon-acetyl-lysine
GGSRGXRNNFXTEEYC

SEQ ID NO: 239 - protein, 14 aa, Artificial Sequence
  Synthetic Construct
  MOD_RES (4)..(4): Xaa is N epsilon-acetyl-lysiine
  MOD_RES (9)..(9): Xaa is N epsilon-acetyl-lysiine
SRGXRNNFXTEEYC

SEQ ID NO: 240 - protein, 12 aa, Artificial Sequence
  Synthetic Construct
  MOD_RES (2)..(2): Xaa is N epsilon-acetyl-lysine
  MOD_RES (7)..(7): Xaa is N epsilon-acetyl-lysine
GXRNNFXTEEYC

SEQ ID NO: 241 - protein, 11 aa, Artificial Sequence
  Synthetic Construct
  MOD_RES (1)..(1): Xaa is N epsilon-acetyl-lysine
  MOD_RES (6)..(6): Xaa is N epsilon-acetyl-lysine
XRNNFXTEEYC

SEQ ID NO: 242 - protein, 11 aa, Artificial Sequence
  Synthetic Construct
  MOD_RES (1)..(1): Xaa is D-N epsilon-acetyl-lysine
  MOD_RES (2)..(2): Xaa is D-Arg
  MOD_RES (5)..(5): Xaa is D-Phe
  MOD_RES (6)..(6): Xaa is N epsilon-acetyl-lysine
XXNNXXTEEYC

SEQ ID NO: 243 - protein, 11 aa, Artificial Sequence
  Synthetic Construct
  MOD_RES (1)..(1): Xaa is D-N epsilon-acetyl-lysine
  MOD_RES (2)..(2): Xaa is D-Arg
  MOD_RES (5)..(5): Xaa is D-Phe
  MOD_RES (6)..(6): Xaa is D-N epsilon-acetyl-lysine
XXNNXXTEEYC

SEQ ID NO: 244 - protein, 11 aa, Artificial Sequence
  Synthetic Construct
  MOD_RES (1)..(1): Xaa is D-N epsilon-acetyl-lysine
  MOD_RES (2)..(2): Xaa is D-Arg
  MOD_RES (5)..(5): Xaa is D-Phe
  MOD_RES (6)..(6): Xaa is D-N epsilon-acetyl-lysine
  MOD_RES (10)..(10): Xaa is D-Tyr
XXNNXXTEEXC

SEQ ID NO: 245 - protein, 8 aa, Artificial Sequence
  Synthetic Construct
  MOD_RES (1)..(1): Xaa is N epsilon-acetyl-lysine
  MOD_RES (6)..(6): Xaa is N epsilon-acetyl-lysine
XRNNFXYC

SEQ ID NO: 246 - protein, 8 aa, Artificial Sequence
  Synthetic Construct
  MOD_RES (1)..(1): Xaa is D-N epsilon-acetyl-lysine
  MOD_RES (2)..(2): Xaa is D-Arg
  MOD_RES (5)..(5): Xaa is D-Phe
  MOD_RES (6)..(6): Xaa is N epsilon-acetyl-lysine
XXNNXXYC

SEQ ID NO: 247 - protein, 8 aa, Artificial Sequence
  Synthetic Construct
  MOD_RES (1)..(1): Xaa is D-N epsilon-acetyl-lysine
  MOD_RES (2)..(2): Xaa is D-Arg
  MOD_RES (5)..(5): Xaa is D-Phe
  MOD_RES (6)..(6): Xaa is D-N epsilon-acetyl-lysine
XXNNXXYC

SEQ ID NO: 248 - protein, 19 aa, Artificial Sequence
  Synthetic Construct
  MOD_RES (8)..(8): Xaa is D-Arg
  MOD_RES (10)..(10): Xaa is D-N epsilon-acetyl-lysine
  MOD_RES (11)..(11): Xaa is D-Arg
  MOD_RES (15)..(15): Xaa is D-N epsilon-acetyl-lysine
TFFYGGSXGXXNNFXTEEY

SEQ ID NO: 249 - protein, 8 aa, Artificial Sequence
  Synthetic Construct
  MOD_RES (1)..(1): Xaa is D-N epsilon-acetyl-lysine
  MOD_RES (2)..(2): Xaa is D-Arg
  MOD_RES (5)..(5): Xaa is D-Phe
  MOD_RES (6)..(6): Xaa is D-N epsilon-acetyl-lysine
  MOD_RES (7)..(7): Xaa is D-Tyr
XXNNXXXC

SEQ ID NO: 250 - protein, 550 aa, Homo sapiens
MPPPRTGRGLLWLGLVLSSVCVALGSETQANSTTDALNVLLIIVDDLRPSLGCYGDKLVRSPNIDQLASHSLLFQNAFAQQAVCAPSRVSFLTGRRPDTTRLYDFNSYWRVHAGNFSTIPQYFKENGYVTMSVGKVFHPGISSNHTDDSPYSWSFPPYHPSSEKYENTKTCRGPDGELHANLLCPVDVLDVPEGTLPDKQSTEQAIQLLEKMKTSASPFFLAVGYHKPHIPFRYPKEFQKLYPLENITLAPDPEVPDGLPPVAYNPWMDIRQREDVQALNISVPYGPIPVDFQRKIRQSYFASVSYLDTQVGRLLSALDDLQLANSTIIAFTSDHGWALGEHGEWAKYSNFDVATHVPLIFYVPGRTASLPEAGEKLFPYLDPFDSASQLMEPGRQSMDLVELVSLFPTLAGLAGLQVPPRCPVPSFHVELCREGKNLLKHFRFRDLEEDPYLPGNPRELIAYSQYPRPSDIPQWNSDKPSLKDIKIMGYSIRTIDYRYTVWVGFNPDEFLANFSDIHAGELYFVDSDPLQDHNMYNDSQGGDLFQLLMP

(19) United States
(12) Patent Application Publication
(10) Pub. No.: US 2016/0367691 A1
(43) Pub. Date: Dec. 22, 2016

(54) TARGETED ENZYME COMPOUNDS AND USES THEREOF

(71) Applicant: ANGIOCHEM INC., Montréal (CA)

(72) Inventors: Michel DEMEULE, Beaconsfield (CA); Sasmita TRIPATHY, Pierrefonds (CA); Alain LAROCQUE, Saint-Laurent (CA); Joanne Catherine MCGREGOR, Cambridge (GB)

(21) Appl. No.: 14/896,258

(22) PCT Filed: Jun. 6, 2014
(86) PCT No.: PCT/CA2014/050524

§ 371 Date: Dec. 4, 2015

Related U.S. Application Data
(60) Provisional application No. 61/831,947, filed on Jun. 6, 2013.

(51) Int. Cl. A61K 47/48; C12N 9/16; C12N 9/96; C07K 7/08; A61K 9/127

(52) U.S. Cl. CPC A61K 47/48246; C07K 7/08; A61K 9/127; C12N 9/96; C12N 9/16; C12Y 301/06013; A61K 38/00

(57) ABSTRACT

The present invention is related to the one-step synthesis of enzyme conjugates and methods for treating or preventing MPS-II by administering such conjugates. In certain embodiments, these compounds can cross the blood-brain barrier or accumulate in the lysosome more effectively than the enzyme alone.

### CROSS-REFERENCE TO RELATED APPLICATIONS

This application claims benefit of U.S. Provisional Application No. 61/831,947, filed Jun. 6, 2013, which is hereby incorporated by reference in its entirety.

### BACKGROUND OF THE INVENTION

Lysosomal storage disorders are group of about 50 rare genetic disorders in which a subject has a defect in a lysosomal enzyme that is required for proper metabolism. These diseases typically result from autosomal or X-linked recessive genes. As a group, the incidence of these disorders is about 1:5000 to 1:10,000.

Hunter syndrome or mucopolysaccharidosis Type II (MPS-II) results from a deficiency of iduronate-2-sulfatase (IDS; also known as idursulfase), an enzyme that is required for lysosomal degradation of heparan sulfate and dermatan sulfate. Because the disorder is X-linked recessive, it primarily affects males. Those with the disorder are unable to break down and recycle these mucopolysaccharides, which are also known as glycosaminoglycans or GAG. This deficiency results in the buildup of GAG throughout the body, which has serious effects on the nervous system, joints, and various organ systems including heart, liver, and skin. There are also a number of physical symptoms, including coarse facial features, enlarged head and abdomen, and skin lesions. In the most severe cases, the disease can be fatal in teen years and is accompanied by severe mental retardation.

There is no cure for MPS-II. In addition to palliative measures, therapeutic approaches have included bone marrow grafts and enzyme replacement therapy. Bone marrow grafts have been observed to stabilize the peripheral symptoms of MPS-II, including cardiovascular abnormalities, hepatosplenomegaly (enlarged liver and spleen), and joint stiffness. This approach, however, does not stabilize or resolve the neuropsychological symptoms associated with this disease (Guffon et al., J. Pediatr. 154:733-7, 2009).

Enzyme replacement therapy by intravenous administration of IDS has also been shown to have benefits, including improvement in skin lesions (Marin et al., Pediatr. Dermatol. 29:369-370, 2012), visceral organ size, gastrointestinal functioning, and reduced need for antibiotics to treat upper airway infections (Hoffman et al., Pediatr. Neurol. 45:181-4, 2011). Like bone marrow grafts, this approach does not improve the central nervous system deficits associated with MPS-II because the enzyme is not expected to cross the blood-brain barrier (BBB; Wraith et al., Eur. J. Pediatr. 1676:267-7, 2008).

Methods for increasing delivery of IDS to the brain have been and are being investigated, including intrathecal delivery (Felice et al., Toxicol. Pathol. 39:879-92, 2011). Intrathecal delivery, however, is a highly invasive technique.

Less invasive and more effective methods of treating MPS-II that address the neurological disease symptoms, in addition to the other symptoms, would therefore be highly desirable.

### SUMMARY OF THE INVENTION

The present invention relates to the discovery of a method for the one-step synthesis of enzyme conjugates (e.g., IDS conjugates) utilizing targeting peptides or peptidomimetics containing a single primary amino group capable of reacting with N-hydroxysuccinimide. Primary amino groups capable of reacting with N-hydroxysuccinamide are primary amino groups (R—NH₂, wherein R is an optionally substituted alkyl or an optionally substituted aromatic group) that exhibit a pKa greater than or equal to 9 and which have a structure that is not stabilized by resonance. In certain embodiments the single reactive primary amino group is the N-terminal primary amino group or may be an ε-amino group on a lysine residue side chain.

Where the primary amino group capable of reacting with N-hydroxysuccinimide is an ε-amino group on a lysine residue side chain, the N-terminal amine of the targeting moiety is protected such that it is incapable of reaction with N-hydroxysuccinimide. Other reactive primary amino groups present in the targeting moiety (e.g. ε-amino group on other lysine residues) are also protected such that they are incapable of reaction with N-hydroxysuccinimide. Where the primary amino group capable of reacting with N hydroxysuccinimide is the N-terminal primary amino group, all other reactive primary amino groups present in the targeting moiety (e.g. ε-amino group on any lysine residues present) are also protected such that they are incapable of reaction with N-hydroxysuccinimide.

IDS conjugates containing these targeting moieties, methods for their production, and methods for treating or preventing MPS-II by administering such IDS conjugates are disclosed. Because these IDS conjugates are capable of crossing the BBB, they can treat not only the peripheral disease symptoms, but may also be effective in treating CNS symptoms. In addition, because the targeting moieties present in the conjugates are capable of targeting the conjugates to the lysosomes, it is expected that these conjugates are more effective than IDS by itself.

Accordingly, in a first aspect, the invention features a compound including a peptidiomimetic having the amino acid sequence of formula (I):

X1-X2-X3-X4-X5-X6-X7-X8-X9-X10-X11-X12-X13-X14-X15-X16-X17-X18-X19 Formula (I)

wherein X1-X19 (e.g., X1-X6, X8, X9, X11-X14, and X16-X19) are, independently, any amino acid (e.g., a naturally or non-naturally occurring amino acid) or absent provided that at least one amino acid is N_{ε}-protected lysine; wherein the amino acid sequence has at least 70% identity to any one of SEQ ID NO: 1-69, 71-73, 75-105 and 107-216; and wherein said amino acid sequence contains a single reactive primary amine group (e.g., a N-terminal primary amine or an unprotected lysine side chain primary amine group).

In some embodiments of the compound includes the amino acid sequence of formula (I), wherein one or more (e.g., two or more, three or more, four or more) amino acids are D-amino acids. In particular embodiments, X8 is a D-amino acid. In certain embodiments, X10 is a D-amino acid. In some embodiments, X11 is a D-amino acid. In other embodiments, X15 is a D-amino acid. In certain embodiments, more than one (e.g., two, three, or four) of X8, X10, X11 and X15 are D-amino acids. In one embodiment, X8, X10 and X11 are D-amino acids. In another embodiment, X8, X10, X11 and X15 are all D-amino acids.

In some embodiments, the residues from X1 through X19, inclusive, are substantially identical to the amino acid sequence of SEQ ID NO:97. In certain embodiments, the residues from X1 through X19, inclusive, are substantially identical to the amino acid sequence of SEQ ID NO:97 and one or more (e.g., two, three, four) amino acids are substituted with the corresponding D-amino acid. In some embodiments, Arg⁸ is substituted with D-Arg. In other embodiments, N_{ε}-protected Lys¹⁰ is substituted with D-N_{ε}-protected Lys. In certain embodiments, Arg¹¹ is substituted with D-Arg. In some embodiments, N_{ε}-protected Lys¹⁵ is substituted with D-N_{ε}-protected Lys. In other embodiments, Arg⁸, N_{ε}-protected Lys¹⁰, Arg¹¹, and N_{ε}-protected Lys¹⁵ have been substituted with the corresponding D-amino acid.

In some embodiments, the peptidomimetic consists of the sequence of formula (I).

In one embodiment, the invention provides a compound including a peptidomimetic having the amino acid sequence of formula I(a):

(N_{ε}-p)Lys-Arg-X3-X4-X5-(N_{ε}p)Lys Formula I(a)

wherein (N_{ε}-p)Lys is an N_{ε}-protected lysine, X3 is Asn or Gln, X4 is Asn or Gln, and X5 is Phe, Tyr, or Trp; wherein the amino acid sequence optionally includes one or more D-isomers of any of the amino acids recited in formula Ia; and wherein said amino acid sequence contains a single reactive primary amine group (e.g., a N-terminal primary amine or an unprotected lysine side chain primary amine group).

In one embodiment of the compound including the amino acid sequence of formula I(a), one or more (e.g., two or more, three or more, four or more) amino acids are D-amino acids. In some embodiments, more than one of the (N_{ε}-p)Lys and Arg residues are D-amino acids. In certain embodiments, the two (N_{ε}-p)Lys residues and the Arg residue are all D-amino acids.

In another embodiment, the invention provides a compound including a peptidomimetic having the amino acid sequence of formula I(b):

Z1-(N_{ε}p)Lys-Arg-X3-X4-X5-(N_{ε}p)Lys-Z2 Formula I(b)
wherein (N_{ε}-p)Lys is an N_{ε}-protected Lys, X3 is Asn or Gln, X4 is Asn or Gln, X5 is Phe, Tyr, or Trp, Z1 is absent, Cys, Gly, Cys-Gly, Arg-Gly, Cys-Arg-Gly, Ser-Arg-Gly, Cys-Ser-Arg-Gly, Gly-Ser-Arg-Gly, Cys-Gly-Ser-Arg-Gly, Gly-Gly-Ser-Arg-Gly, Cys-Gly-Gly-Ser-Arg-Gly, Tyr-Gly-Gly-Ser-Arg-Gly, Cys-Tyr-Gly-Gly-Ser-Arg-Gly, Phe-Tyr-Gly-Gly-Ser-Arg-Gly, Cys-Phe-Tyr-Gly-Gly-Ser-Arg-Gly, Phe-Phe-Tyr-Cys-Phe-Phe-Tyr-Gly-Gly-Ser-Arg-Gly, Thr-Phe-Phe-Tyr-Gly-Gly-Ser-Arg-Gly, or Cys-Thr-Phe-Phe-Tyr-Gly-Gly-Ser-Arg-Gly, and Z2 is absent, Cys, Tyr, Tyr-Cys, Cys-Tyr, Thr-Glu-Glu-Tyr, or Thr-Glu-Glu-Tyr-Cys; wherein the amino acid sequence optionally includes one or more D-isomers of an amino acid recited in formula Ib, X3, X4, X5, Z1, or Z2; and wherein said amino acid sequence contains a single reactive primary amine group (e.g., a N-terminal primary amine or an unprotected lysine side chain primary amine group).

In one embodiment of the compound including the amino acid sequence of formula I(b), one or more (e.g., two or more, three or more, four or more) amino acids are D-amino acids. In certain embodiments, more than one of the (N_{ε}-p)Lys and Arg residues are D-amino acids. In some embodiments, the two (N_{ε}-p)Lys residues and the Arg residue are all D-amino acids.

In yet another embodiment, the invention provides a compound including a peptidomimetic having an amino acid sequence of formula I(c):

X1-X2-Asn-Asn-X5-X6 Formula I(c)
wherein X1 is N_{ε}-protected Lys or D-N_{ε}-protected Lys, X2 is Arg or D-Arg, X5 is Phe or D-Phe, and X6 is N_{ε}-protected Lys or D-N_{ε}-protected Lys; wherein at least one (e.g., at least two, three, or four) of the amino acids recited in formula Ic, X1, X2, X5, or X6 is a D-amino acid, and wherein said amino acid sequence contains a single reactive primary amine group (e.g., a N-terminal primary amine or an unprotected lysine side chain primary amine group).

In one embodiment of the compound including the amino acid sequence of formula I(c), two or more (e.g. three or more, four or more) amino acids are D-amino acids. In certain embodiments, more than one of X1, X2 and X6 are D-amino acids. In some embodiments, X1, X2 and X6 are all D-amino acids.

In a further embodiment, the invention provides a compound including a peptidomimetic having an amino acid sequence of formula I(d):

X1-X2-Asn-Asn-X5-X6-X7 Formula I(d)
wherein X1 is N_{ε}-protected Lys or D-N_{ε}-protected Lys; X2 is Arg or D-Arg; X5 is Phe or D-Phe; X6 is N_{ε}-protected Lys or D-N_{ε}-protected Lys, and X7 is Tyr or D-Tyr; wherein at least one (e.g., at least two, three, four, or five) of the amino acids recited in formula Id, X1, X2, X5, X6, or X7 is a D-amino acid; and wherein said amino acid sequence contains a single reactive primary amine group (e.g., a N-terminal primary amine or an unprotected lysine side chain primary amine group).

In one embodiment of the compound including the amino acid sequence of formula I(d), two or more (e.g. three or more, four or more) amino acids are D-amino acids. In some embodiments, more than one of X1, X2 and X6 are D-amino acids. In certain embodiments, X1, X2 and X6 are all D-amino acids.

In another embodiment, the invention provides a compound including a peptidomimetic having an amino acid sequence of formula I(e):

Z1-X1-X2-Asn-Asn-X5-X6-X7-Z2 Formula I(e)
wherein X1 is N_{ε}-protected Lys or D-N_{ε}-protected Lys, X2 is Arg or D-Arg, X5 is Phe or D-Phe, X6 is N_{ε}-protected Lys or D-N_{ε}-protected Lys, X7 is Tyr or D-Tyr, Z1 is absent, Cys, Gly, Cys-Gly, Arg-Gly, Cys-Arg-Gly, Ser-Arg-Gly, Cys-Ser-Arg-Gly, Gly-Ser-Arg-Gly, Cys-Gly-Ser-Arg-Gly, Gly-Gly-Ser-Arg-Gly, Cys-Gly-Gly-Ser-Arg-Gly, Tyr-Gly-Gly-Ser-Arg-Gly, Cys-Tyr-Gly-Gly-Ser-Arg-Gly, Phe-Tyr-Gly-Gly-Ser-Arg-Gly, Cys-Phe-Tyr-Gly-Gly-Ser-Arg-Gly, Phe-Phe-Tyr-Gly-Gly-Ser-Arg-Gly, Cys-Phe-Phe-Tyr-Gly-Gly-Ser-Arg-Gly, Thr-Phe-Phe-Tyr-Gly-Gly-Ser-Arg-Gly, or Cys-Thr-Phe-Phe-Tyr-Gly-Gly-Ser-Arg-Gly, and Z2 is absent, Cys, Tyr, Tyr-Cys, Cys-Tyr, Thr-Glu-Glu-Tyr, or Thr-Glu-Glu-Tyr-Cys; wherein at least one (e.g., at least two, three, or four) of the amino acids recited in formula Ie, X1, X2, X5, X6 or X7 is a D-amino acid; wherein the peptidomimetic optionally includes one or more D-isomers of an amino acid recited in Z1 or Z2; and wherein said amino acid sequence contains a single reactive primary amine group (e.g., a N-terminal primary amine or an unprotected lysine side chain primary amine group).

In one embodiment of the compound including the amino acid sequence of formula I(e), two or more (e.g. three or more, four or more) amino acids are D-amino acids. In some embodiments, more than one of X1, X2 and X6 are D-amino acids. In certain embodiments, X1, X2 and X6 are all D-amino acids. In other embodiments, the amino acid sequence further includes one or more D-isomers of an amino acid recited in Z1.

In one aspect, the invention provides a compound comprising an amino acid sequence having the following formula:

X1-X2-X3-X4-X5-X6-X7-X8-X9-X10-X11-X12-X13-X14-X15-X16-X17-X18-X19 Formula I
wherein X1-X19 are any amino acid or are absent;
at least one amino acid is N_{ε}-protected lysine; and
said amino acid sequence has at least 70% identity to any one of SEQ ID NO:97, 1-69, 71-73, 75-96, 98-105, and 107-216 or has the amino acid sequence of any one of formula Ia-Ie wherein the N_{ε}-protected lysine residues present in formula Ia-Ie are Nε-(acetyl)-L-lysine; and
wherein said amino acid sequence contains a single reactive primary amine group.

In certain embodiments of any of the compounds including the amino acid sequences of formulae I (a-e), the amino acid sequence is 19 amino acids or fewer in length (e.g., fewer than 19, 18, 17, 16, 15, 14, 12, 10, 11, 8, or 7 amino acids, or any range between these numbers). In certain embodiments, the compounds of the invention are capable of being transported to the lysosome or across the blood brain barrier. When conjugated to enzymes, the peptidomimetics of the invention act as targeting moieties directing the enzymes to the lysosome and/or across the blood brain barrier.

In a second aspect, the invention provides enzyme conjugates in which these peptidomimetics act as targeting moieties. Related amino acid sequences that do not contain N_{ε}-protected lysine residues may also act as targeting moieties if said amino acid sequence contains a single reactive primary amine group (e.g. a sequence that does not contain any lysine residues). Accordingly, the invention provides a compound of formula II:

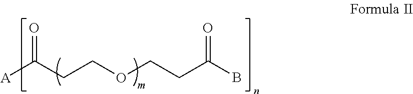

wherein A is an enzyme, an enzyme fragment that retains the activity of said enzyme, or an enzyme analog that exhibits enzymatic activity, wherein said enzyme, enzyme fragment, or enzyme analog is attached via one or more NH groups derived from the reaction of a primary amine group;
   m is an integer between 1 and 45, e.g., 1 to 10, 2 to 9, 2 to 13, 5 to 10, 2 to 7, 5 to 15, 10 to 20, 15 to 25, 20 to 30, 25 to 35, 30 to 40, or 35 to 45 (e.g., m is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or 45);
n is an integer between 1 and 6, e.g. 1 to 4, 3 to 6, 2 to 5, 1 to 3, 2 to 4, 3 to 5, or 4 to 6 (e.g., n is 1, 2, 3, 4, 5 or 6); and
B comprises an amino acid sequence that contains a single reactive primary amine group (e.g., a N-terminal primary amine or an unprotected lysine side chain primary amine group) that has either (a) at least 70% identity to any one of SEQ ID NO: 1-69, 71-73, 75-105 and 107-216, or (b) the amino acid sequence of any one of formulae I, Ia, Ib, Ic, Id or Ie, wherein B is attached via an NH group derived from reaction of its single reactive primary amine group; and
wherein the compound exhibits the activity of the enzyme.

In one embodiment, B comprises an amino acid sequence with at least 70% identity to any one of SEQ ID NO:97, 1-69, 71-73, 75-96, 98-105, and 107-216 or has the amino acid sequence of any one of formula Ia-Ie, wherein the N_{ε}-protected lysine residues present in formula Ia-Ie are Nε-(acetyl)-L-lysine), wherein said amino acid sequence contains a single primary amine group and wherein B is attached via an NH group derived from reaction of said primary amine.

The amino acid sequences of formulae I, la, Ib, Ic, Id and Ie are defined above. The skilled person will recognize that the more particular embodiments of these sequences may also be used in the compound of formula II.

In some embodiments, the compound of formula (II) has the structure of formula III:

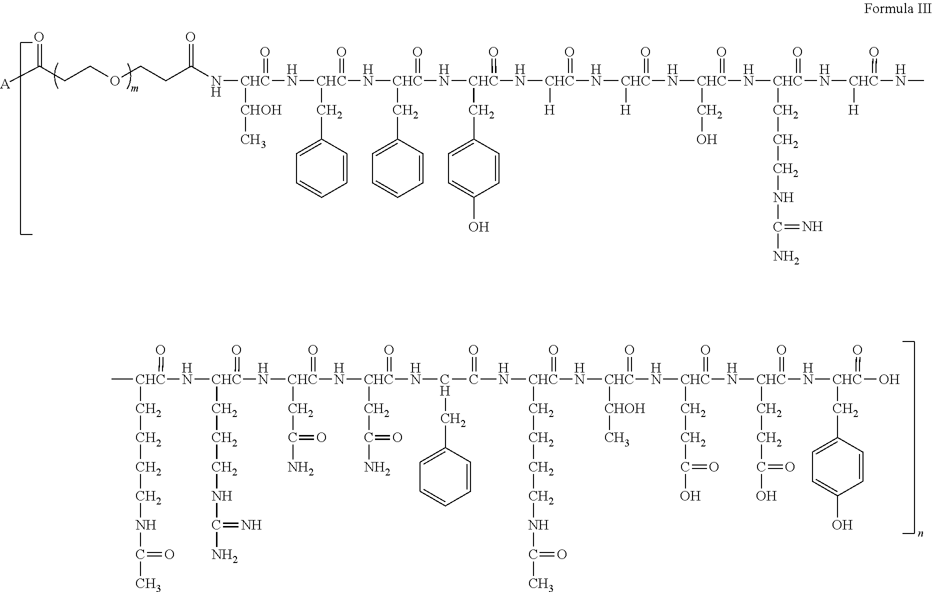

wherein A, m and n are as defined above.

In another aspect, the invention provides a population of compounds of formula IIa:

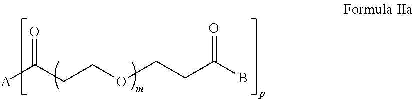

wherein A, B and m are as defined above, and wherein p is between 1 and 6, more particularly between 1 to 3 (e.g., approximately 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, or 3.0).

In a more particular embodiment, the invention provides a population of compounds of formula IIIa:

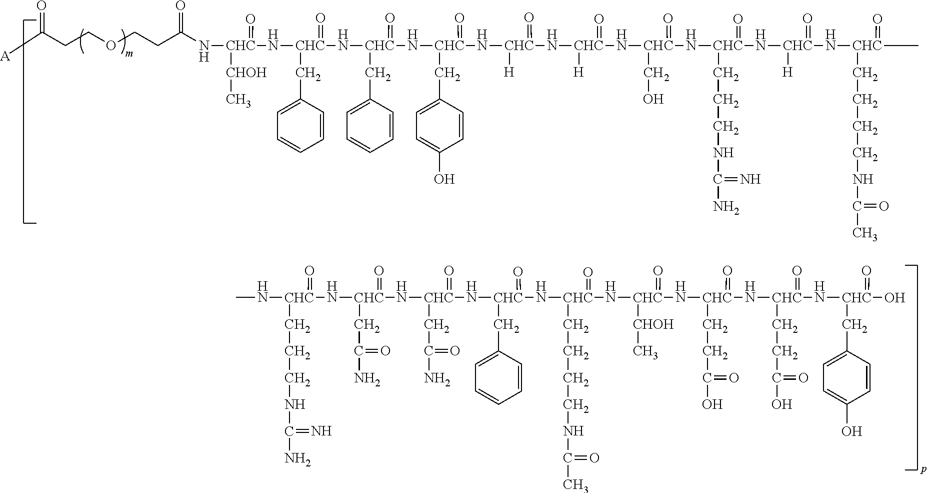

wherein A, B and m are as defined above, and wherein p is between 1 and 6, more particularly between 1 to 3 (e.g., approximately 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, or 3.0).

In certain embodiments relating to the compound of formula III or to a population of compounds of formula IIIa, one or more (e.g., two, three, four) amino acids are substituted with the corresponding D-amino acid. In some embodiments, Arg⁸ is substituted with D-Arg. In other embodiments, Lys¹⁰ is substituted with D-Lys. In certain embodiments, Arg¹¹ is substituted with D-Arg. In some embodiments, Lys¹⁵ is substituted with D-Lys. In other embodiments, Arg⁸, Lys¹⁰, Arg¹¹, and Lys¹⁵ have been substituted with the corresponding D-amino acid.

In another aspect, the invention provides intermediates useful in the production of compounds of formulae II and III. Accordingly, the invention provides a compound of formula IV:

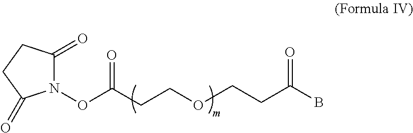

wherein m and B are as defined above.

In certain embodiments, the compound has the structure:

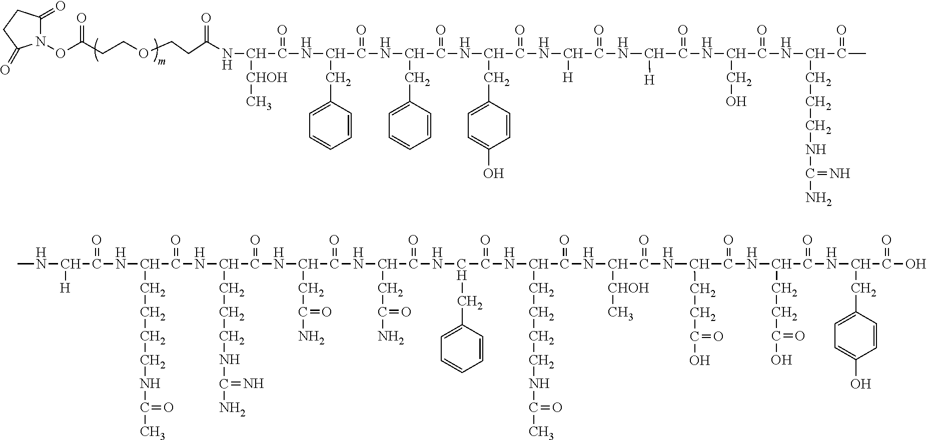

wherein m is as defined above.

In more particular embodiments, one or more (e.g., two, three, four) amino acids are substituted with the corresponding D-amino acid. In some embodiments, Arg⁸ is substituted with D-Arg. In other embodiments, Lys¹⁰ is substituted with D-Lys. In certain embodiments, Arg¹¹ is substituted with D-Arg. In some embodiments, Lys¹⁵ is substituted with D-Lys. In other embodiments, Arg⁸, Lys¹⁰, Arg¹¹, and Lys¹⁵ have been substituted with the corresponding D-amino acid.

In another aspect, the invention provides a compound of formula V:

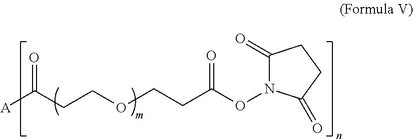

wherein A, m and n are as defined above.

Another aspect of the invention features methods for producing the compound of formula II and populations of formula IIa. A first method includes reacting a compound of formula V with a compound comprising an amino acid sequence that contains a single reactive primary amine group (e.g., a N-terminal primary amine or an unprotected lysine side chain primary amine group) that has either (a) at least 70% identity to any one of SEQ ID NO: 1-69, 71-73, 75-105 and 107-216 or (b) the amino acid sequence of any one of formula I, Ia, Ib, Ic, Id or Ie, under conditions (e.g., pH between 7 and 14, (e.g., 7 to 9, 8 to 10, 9 to 11, 10 to 12, 11 to 13, or 12 to 14)) that produce a compound of formula I.

An alternative method for producing the compound of formula II and populations of formula IIa includes reacting a compound of formula IV with an enzyme, enzyme fragment, or enzyme analog, wherein the enzyme, enzyme fragment, or enzyme analog has one or more primary amine groups under conditions (e.g., pH between 7 and 14) that produce a compound of formula I. In a particular embodiment of this method, B comprises an amino acid sequence with at least 70% identity to any one of SEQ ID NO: 97, 1-69, 71-73, 75-96, 98-105, and 107-216 or has the amino acid sequence of any one of formula Ia-Ie, wherein the N_{ε}-protected lysine residues present in formula Ia-Ie are Nε-(acetyl)-L-lysine), wherein said amino acid sequence contains a single primary amine group, and wherein B is attached via an NH group derived from reaction of said primary amine.

In a more particular embodiment of this method, the compound of formula IV has the structure:

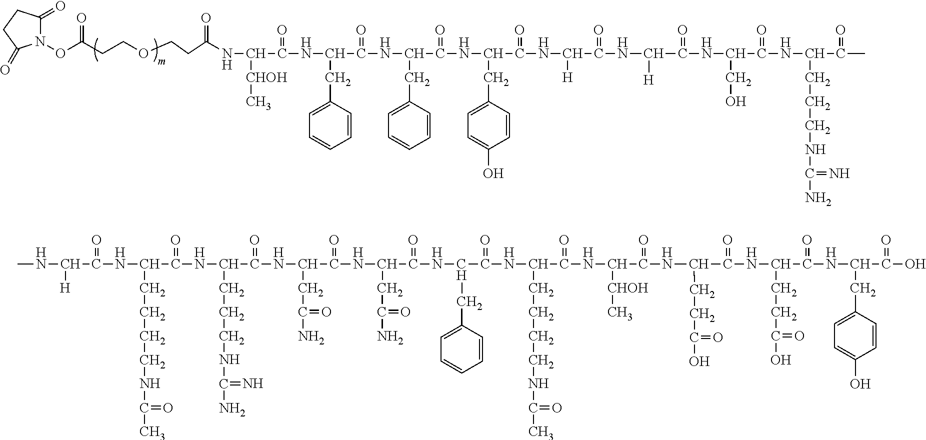

wherein m is as defined above.

In certain embodiments of any of the foregoing compounds, populations, and methods, m is 2. In some embodiments of any of the foregoing compounds, populations, and methods, m is 4. In some embodiments of any of the foregoing compounds, populations, and methods, m is 7. In some embodiments of any of the foregoing compounds, populations, and methods, m is 9. In some embodiments of any of the foregoing compounds, populations, and methods, m is 13.

In some embodiments m is 1 to 13, e.g., 2, 4, 7, 9, or 13 and n is 1 to 3, 2 to 4, 3 to 5, or 4 to 6.

In further embodiments of any of the foregoing compounds and populations, A is iduronate-2-sulfatase (IDS), an IDS fragment having IDS activity, or an IDS analog having IDS activity. In certain embodiments, A is iduronate-2-sulfatase (IDS).

In some embodiments, A is attached via one or more NH groups derived from reaction of a primary amine group of a lysine (e.g., a lysine of IDS, such as, lysine 199, lysine 211, lysine 240, lysine 295, lysine 347, lysine 376, lysine 479, and lysine 483 using the numbering of full length human IDS isoform a).

In some embodiments of any of the foregoing compounds and populations, the amino acid sequence of B is substantially identical to the amino acid sequence of SEQ ID NO:97 or SEQ ID NO.200. In certain embodiments, one or more (e.g., two, three, four) amino acids are substituted with the corresponding D-amino acid. In some embodiments, Arg⁸ is substituted with D-Arg. In other embodiments, N_{ε}-protected Lys (or Ac Lys)¹⁰ is substituted with D-N_{ε}-protected Lys (or Ac Lys). In certain embodiments, Arg¹¹ is substituted with D-Arg. In some embodiments, N_{ε}-protected Lys (or N_{ε}—Ac-Lys)¹⁸ is substituted with D-N_{ε}-protected Lys (or Ac Lys). In other embodiments, Arg⁸, N_{ε}-protected Lys (or N_{ε}—Ac-Lys)¹⁰, Arg¹¹, and N_{ε}-protected Lys (or N_{ε—Ac-Lys)}¹⁸ have been substituted with the corresponding D-amino acid.

In certain embodiments of any of the foregoing compounds or populations, the amino acid sequence of B may be fewer than 20 amino acids in length. In certain embodiments, the amino acid sequence of B may be fewer than 15 amino acids in length.

In certain embodiments of any of the foregoing compounds and populations, the amino acid sequence of B may have a C-terminus that is amidated. In other embodiments of any of the foregoing compounds and populations, the compound or population is efficiently transported across the BBB.

In some embodiments, the compounds of formula II and III of the invention are efficiently transported to the lysosome and/or across the blood brain barrier.

The invention features a composition that includes nanoparticles which are conjugated to the compounds of formula II or III or the populations of formula IIa or IIIa described above. The invention also features a liposome formulation of any of the compounds of formula II or III or of the populations of formula IIa or IIIa featured above.

The invention features a pharmaceutical composition that includes any one of the compounds of formula II or III, or the populations of IIa or IIIa described above and a pharmaceutically acceptable carrier. The invention also features a method of treating or treating prophylactically a subject having a lysosomal storage disorder, where the method includes administering to a subject any of the above described compounds of formula II or III, the populations of formula IIa or IIIa, or compositions. In one aspect of the method, the lysosomal storage disorder is mucopolysaccharidosis Type II (MPS-II). MPS-II may be treated or treated prophylactically with compounds, populations, and compositions of the invention wherein A is iduronate-2-sulfatase (IDS), an IDS fragment having IDS activity, or an IDS analog having IDS activity. In one embodiment, the subject has the severe form of MPS-II. In an alternative embodiment the subject has the attenuated form of MPS-II. In yet another embodiment, the subject has neurological symptoms. The subject can start treatment at less than five years of age, (e.g., under three years of age). The subject can be an infant. The methods of the invention also include parenteral administration of the compounds, populations, and compositions of the invention.

By “subject” is meant a human or non-human animal (e.g., a mammal).

By “lysosomal enzyme” is meant any enzyme that is found in the lysosome in which a defect in that enzyme can lead to a lysosomal storage disorder.

By “lysosomal storage disorder” is meant any disease caused by a defect in a lysosomal enzyme. Approximately fifty such disorders have been identified.

By “treating” a disease, disorder, or condition in a subject is meant reducing at least one symptom of the disease, disorder, or condition by administrating a therapeutic agent to the subject.

By “treating prophylactically” a disease, disorder, or condition in a subject is meant reducing the frequency of occurrence of or reducing the severity of a disease, disorder or condition by administering a therapeutic agent to the subject prior to the onset of disease symptoms.

By a compound which is “efficiently transported across the BBB” is meant a polypeptide that is able to cross the BBB at least as efficiently as Angiopep-6 (i.e., SEQ ID NO:111 wherein AcK is replaced with K) (i.e., greater than 38.5% that of Angiopep-1 (i.e., SEQ ID NO:67 wherein AcK is replaced with K) (250 nM) in the in situ brain perfusion assay described in U.S. patent application Ser. No. 11/807,597, filed May 29, 2007, hereby incorporated by reference). Accordingly, a polypeptide which is “not efficiently transported across the BBB” is transported to the brain at lower levels (e.g., transported less efficiently than Angiopep-6 (i.e., SEQ ID NO:111 wherein NεpK is replaced with K)).

By a polypeptide or compound which is “efficiently transported to a particular cell type” is meant that the polypeptide or compound is able to accumulate (e.g., either due to increased transport into the cell, decreased efflux from the cell, or a combination thereof) in that cell type to at least a 10% (e.g., 25%, 50%, 100%, 200%, 500%, 1,000%, 5,000%, or 10,000%) greater extent than either a control substance, or, in the case of a conjugate, as compared to the unconjugated agent. Such activities are described in detail in International Application Publication No. WO 2007/009229, hereby incorporated by reference.

By “substantial identity” or “substantially identical” is meant a polypeptide or polynucleotide sequence that has the same polypeptide or polynucleotide sequence, respectively, as a reference sequence, or has a specified percentage of amino acid residues or nucleotides, respectively, that are the same at the corresponding location within a reference sequence when the two sequences are optimally aligned. For example, an amino acid sequence that is “substantially identical” to a reference sequence has at least 50%, 60%, 70%, 75%, 80%, 85%, 90%, 95%, 96%, 97%, 98%, 99%, or 100% identity to the reference amino acid sequence. For polypeptides, the length of comparison sequences will generally be at least 5, 6, 7, 8, 9, 10, 11, 12, 13, 14, 15, 16, 17, 18, 19, 20, 25, 50, 75, 90, 100, 150, 200, 250, 300, or 350 contiguous amino acids (e.g., a full-length sequence). For nucleic acids, the length of comparison sequences will generally be at least 5, 10, 11, 12, 13, 14, 15, 16, 17, 18, 19, 20, 21, 22, 23, 24, or 25 contiguous nucleotides (e.g., the full-length nucleotide sequence). Sequence identity may be measured using sequence analysis software on the default setting (e.g., Sequence Analysis Software Package of the Genetics Computer Group, University of Wisconsin Biotechnology Center, 1710 University Avenue, Madison, Wis. 53705). Such software may match similar sequences by assigning degrees of homology to various substitutions, deletions, and other modifications.

Other features and advantages of the invention will be apparent from the following Detailed Description, the drawings, and the claims.

### BRIEF DESCRIPTION OF THE DRAWINGS

FIG. 1 is the amino acid sequence of SEQ ID NO:250.

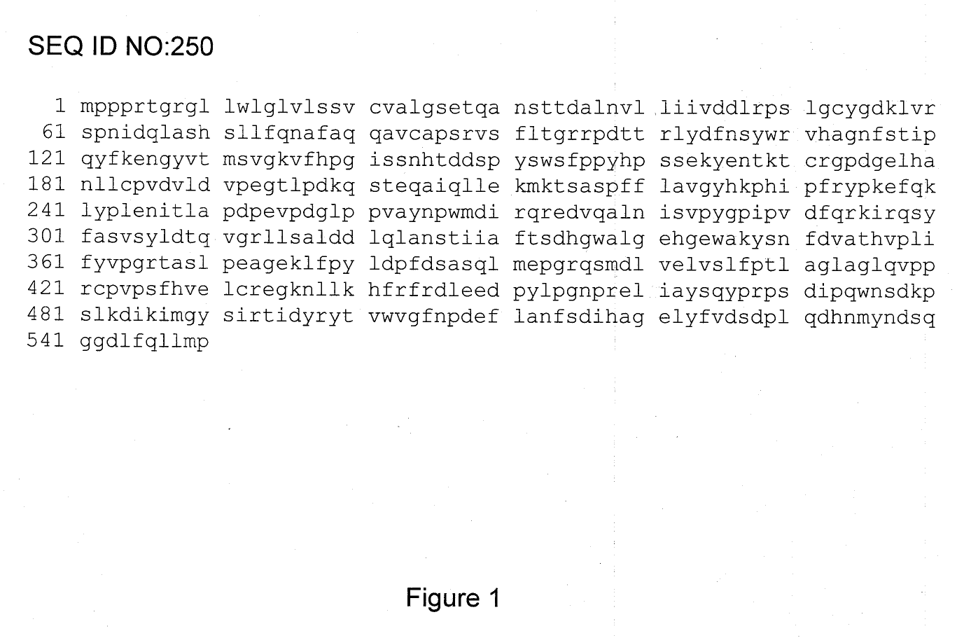

FIG. 2 is an image of the HPLC trace of the reaction between AN2[(Ac)Lys^{10,15}] and Bis-diPEG-NHS Ester. FIG. 2A is the HPLC trace of the reaction before purification and FIG. 2B is the HPLC trace after purification.

FIG. 3 is an image of the HLPC-MS trace of AN2[(Ac)Lys^{10,15}]-Peg-NHS Ester after Lyophilization. FIG. 3A is the HPLC trace and FIG. 3B is the MS trace.

FIG. 4 is an image of the SP HPLC analysis of 77-18-1.

FIG. 5 is an image of the SEC HPLC analysis of 77-18-1.

FIG. 6 is an image of the MALDI TOF analysis of 77-18-1.

FIG. 7 is an image of the SP HPLC analysis of 77-18-2.

FIG. 8 is an image of the SEC HPLC analysis of 77-18-2.

FIG. 9 is an image of the MALDI TOF analysis of 77-18-2.

FIG. 10 is an image of the SP HPLC analysis of 77-18-3.

FIG. 11 is an image of the SEC HPLC analysis of 77-18-3.

FIG. 12 is an image of the MALDI TOF analysis of 77-18-3.

FIG. 13 is a graph illustrating the in vitro enzymatic activity of 77-18-3 in comparison to JR-032.

FIG. 14 is a graph illustrating the brain perfusion of 77-18-3 compared to JR-032.

FIG. 15 is a graph illustrating the GAG reduction in MPSII cells by ANG3402, ANG3403, and ANG305 in comparison to JR-032.

FIG. 16 is a graph illustrating plasma levels over time of ANG3405.

FIG. 17 is a graph illustrating brain distribution at 1 hour of ANG3402, ANG3403, and ANG3405.

FIG. 18 is a graph illustrating in vivo reduction of GAG levels by ANG3402, ANG3403, and ANG3405.

### DETAILED DESCRIPTION

The present invention relates to enzyme conjugates and their production utilizing a newly discovered one-step method employing polypeptides which have only one reactive primary amine group. Methods for their production and methods for the treatment of MPS-II by administering such compounds are also disclosed. The compounds are capable of targeting the lysosome and/or crossing the BBB. Such compounds are exemplified by IDS-peptide conjugates. These proteins maintain IDS enzymatic activity both in an enzymatic assay and in a cellular model of MPS-II. Accordingly, we believe that these compounds can increase enzyme concentrations in the lysosome, thus resulting in more effective therapy, particular in tissues and organs that express an Angiopep-2 receptor, (e.g., the LRP-1 receptor), such as liver, kidney, and spleen.

These features overcome some of the biggest disadvantages of current therapeutic approaches because intravenous administration of IDS by itself does not treat CNS disease symptoms. In contrast to physical methods for bypassing the BBB, such intrathecal or intracranial administration, which are highly invasive and thus generally an unattractive solution to the problem of CNS delivery, the present invention allows for noninvasive brain delivery. In addition, improved transport of the therapeutic to the lysosomes may allow for reduced dosing or reduced frequency of dosing, as compared to standard enzyme replacement therapy.

### Lysosomal Storage Disorders

Lysosomal storage disorders are a group of disorders in which the metabolism of lipids, glycoproteins, or mucopolysaccharides is disrupted based on enzyme dysfunction. This dysfunction leads to cellular buildup of the substance that cannot be properly metabolized. Symptoms vary from disease to disease, but problems in the organ systems (liver, heart, lung, and spleen), bones, as well as neurological problems are present in many of these diseases. Typically, these diseases are caused by rare genetic defects in the relevant enzymes. Most of these diseases are inherited in autosomal recessive fashion, but some, such as MPS-II, are X-linked recessive diseases.

### Lysosomal Enzymes

The conjugates of the invention may comprise any lysosomal enzyme known in the art that is useful for treating a lysosomal storage disorder. In certain embodiments, the conjugates of the invention comprise iduronate-2-sulfatase (IDS; also known as idursulfase) and retain sulfatase activity. The conjugates may include IDS, a fragment of IDS that retains enzymatic activity, or an IDS analog which exhibits enzymatic activity.

To test whether particular fragment or analog has enzymatic activity, the skilled artisan can use any appropriate assay. Assays for measuring IDS activity, for example, are known in art, including those described in Hopwood, Carbohydr. Res. 69:203-16, 1979, Bielicki et al., Biochem. J. 271:75-86, 1990, and Dean et al., Clin. Chem. 52:643-9, 2006. A similar fluorometric assay is also described in the examples. Using any of these assays, the skilled artisan would be able to determine whether a particular IDS fragment or analog has enzymatic activity. These assays can also identify whether polypeptides or peptidomimetics have enzyme activity.

Three human isoforms of IDS are known, isoforms a, b, and c. Isoform a is a 550 amino acid protein, isoform b is a 343 amino acid protein which has a different C-terminal region as compared to the longer isoform a, and isoform c has changes at the N-terminal due to the use of a downstream start codon. Any of these isoforms may be used in the compounds of the invention. Recombinant iduronate-2-sulfatase enzymes (e.g., JR-032) are known in the art. JR-032 is a recombinant human IDS full length isoform a (INN: idursulfase) manufactured as described in U.S. Pat. No. 5,932,211.

In certain embodiments, the IDS or the IDS fragment has the amino acid sequence of human IDS isoform a or a fragment thereof (e.g., amino acids 26-550 of isoform a, which represents the mature form of isoform a). Where an IDS fragment is used, this may be at least 50, 100, 150, 200, 250, 300, 350, 400, 450, or 500 amino acids in length.

IDS analogs have a different amino acid sequence to the human isoforms of IDS (or fragments thereof). IDS analogs may have sequences that are substantially identical (e.g., at least 60%, 70%, 80%, 85%, 90%, 95%, 96%, 97%, 98%, or 99% identical) to the sequence of human IDS isoform a, isoform b, isoform c, or to amino acids 26-550 of isoform a. In some embodiments, the analogs may contain just naturally occurring amino acids.

Naturally occurring residues are divided into groups based on common side chain properties:
(1) hydrophobic: norleucine, methionine (Met), Alanine (Ala), Valine (Val), Leucine (Leu), Isoleucine (Ile), Histidine (His), Tryptophan (Trp), Tyrosine (Tyr), Phenylalanine (Phe),
(2) neutral hydrophilic: Cysteine (Cys), Serine (Ser), Threonine (Thr)
(3) acidic/negatively charged: Aspartic acid (Asp), Glutamic acid (Glu)
(4) basic: Asparagine (Asn), Glutamine (Gin), Histidine (His), Lysine (Lys), Arginine (Arg)
(5) residues that influence chain orientation: Glycine (Gly), Proline (Pro);
(6) aromatic: Tryptophan (Trp), Tyrosine (Tyr), Phenylalanine (Phe), Histidine (His),
(7) polar: Ser, Thr, Asn, Gln
(8) basic positively charged: Arg, Lys, His, and;
(9) charged: Asp, Glu, Arg, Lys, His

Analogs may be generated by substitutional mutagenesis. To retain enzymatic activity, amino acids are typically substituted with others falling within the same group. Such substitutions are referred to as “conservative”. Examples of substitutions identified as “conservative substitutions” are shown in Table 1. If such substitutions result in a change not desired, then other type of substitutions, denominated “exemplary substitutions” in Table 1 may be considered.

Other amino acid substitutions are listed in Table 1.

TABLE 1
Amino acid substitutions

| Original | | Conservative |
|---|---|---|
| residue | Exemplary substitution | substitution |
| Ala (A) | Val, Leu, Ile | Val |
| Arg (R) | Lys, Gln, Asn | Lys |
| Asn (N) | Gln, His, Lys, Arg | Gln |
| Asp (D) | Glu | Glu |
| Cys (C) | Ser | Ser |
| Gln (Q) | Asn | Asn |
| Glu (E) | Asp | Asp |
| Gly (G) | Pro | Pro |
| His (H) | Asn, Gln, Lys, Arg | Arg |
| Ile (I) | Leu, Val, Met, Ala, Phe, norleucine | Leu |
| Leu (L) | Norleucine, Ile, Val, Met, Ala, Phe | Ile |
| Lys (K) | Arg, Gln, Asn | Arg |
| Met (M) | Leu, Phe, Ile | Leu |
| Phe (F) | Leu, Val, Ile, Ala | Leu |
| Pro (P) | Gly | Gly |
| Ser (S) | Thr | Thr |
| Thr (T) | Ser | Ser |
| Trp (W) | Tyr | Tyr |
| Tyr (Y) | Trp, Phe, Thr, Ser | Phe |
| Val (V) | Ile, Leu, Met, Phe, Ala, norleucine | Leu |

In other embodiments, IDS analogs may include non-naturally occurring amino acids or other chemical modifications such that the IDS analog is a peptidomimetic. Chemical modifications that may be employed to form peptidomimetic IDS analogs are described in more detail below. The skilled reader will appreciate that the targeting peptidomimetic or B may contain any of these chemical modifications.

Targeting Moieties

In some embodiments of any of the foregoing compounds and populations, the amino acid sequence of the targeting peptidomimetic or B may include an amino acid sequence that is substantially identical to any of SEQ ID NOS:1-69, 71-73, 73-105 and 107-216 (e.g., Acetylated-Angiopep-2 (SEQ ID NO:200)).

As discussed in the section entitled Summary of the Invention, compound including peptidomimetics having the amino acid sequence of formula I or formulae I(a-e) may be capable of being transported to the lysosome or across the blood brain barrier. Each of these peptidomimetics has a single reactive primary amine group through which it can be conjugated to an enzyme to form compounds of formulae II or III. When conjugated to enzymes in these compounds, the peptidomimetic moiety is referred to as a targeting moiety or B and serves to direct the compounds to the lysosome and/or across the blood brain barrier. The skilled person will appreciate that any disclosure relating to the peptidomimetic is equally applicable to B.

In particular embodiments, the peptidomimetic or B includes the amino acid sequence of the formula (Ac)Lys-Arg-X3-X4-X5-(Ac)Lys (formula Ia), ((Ac)Lys is N_{ε}-(acetyl)-L-lysine), where X3 is Asn or Gln; X4 is Asn or Gln; and X5 is Phe, Tyr, or Trp, where the amino acid sequence optionally includes one or more D-isomers of an amino acid recited in formula Ia. In certain embodiments of formula Ia, the amino acid sequence is optionally fewer than 200 amino acids in length (e.g., fewer than 150, 100, 75, 50, 45, 40, 35, 30, 25, 20, 19, 18, 17, 16, 15, 14, 12, 10, 11, 8, or 7 amino acids, or any range between these numbers). In other embodiments, the amino acid sequence 19 amino acids or fewer in length (e.g., fewer than 19, 18, 17, 16, 15, 14, 12, 10, 11, 8, or 7 amino acids, or any range between these numbers. In one embodiment of the amino acid sequence of formula I(a), one or more (e.g., two or more, three or more, four or more) amino acids are D-amino acids. In certain embodiments, more than one of the (N_{ε}—Ac)Lys and Arg residues are D-amino acids. In some embodiments, the two (N_{ε}—Ac)Lys and Arg residues are all D-amino acids.

In other embodiments, the amino acid sequence of the targeting peptidomimetic or B includes the formula Z1-(Ac)Lys-Arg-X3-X4-X5-(Ac)Lys-Z2 (formula Ib), where X3 is Asn or Gln; X4 is Asn or Gln; X5 is Phe, Tyr, or Trp; Z1 is absent, Cys, Gly, Cys-Gly, Arg-Gly, Cys-Arg-Gly, Ser-Arg-Gly, Cys-Ser-Arg-Gly, Gly-Ser-Arg-Gly, Cys-Gly-Ser-Arg-Gly, Gly-Gly-Ser-Arg-Gly, Cys-Gly-Gly-Ser-Arg-Gly, Tyr-Gly-Gly-Ser-Arg-Gly, Cys-Tyr-Gly-Gly-Ser-Arg-Gly, Phe-Tyr-Gly-Gly-Ser-Arg-Gly, Cys-Phe-Tyr-Gly-Gly-Ser-Arg-Gly, Phe-Phe-Tyr-Gly-Gly-Ser-Arg-Gly, Cys-Phe-Phe-Tyr-Gly-Gly-Ser-Arg-Gly, Thr-Phe-Phe-Tyr-Gly-Gly-Ser-Arg-Gly, or Cys-Thr-Phe-Phe-Tyr-Gly-Gly-Ser-Arg-Gly; and Z2 is absent, Cys, Tyr, Tyr-Cys, Cys-Tyr, Thr-Glu-Glu-Tyr, or Thr-Glu-Glu-Tyr-Cys; and where the amino acid sequence optionally includes one or more D-isomers of an amino acid recited in formula Ib, Z1, or Z2. In any of the above aspects, the amino acid sequence of the targeting peptidomimetic or B may include the amino acid sequence (Ac)Lys-Arg-Asn-Asn-Phe-(Ac)Lys. In other embodiments, the amino acid sequence of the targeting peptidomimetic or B has an amino acid sequence of (Ac)Lys-Arg-Asn-Asn-Phe-(Ac)Lys-Tyr. In still other embodiments, the amino acid sequence of the targeting peptidomimetic or B has an amino acid sequence of (Ac)Lys-Arg-Asn-Asn-Phe-(Ac)Lys-Tyr-Cys.

In one embodiment of the amino acid sequence of formula I(b), one or more (e.g., two or more, three or more, four or more) amino acids are D-amino acids. In particular embodiments, more than one of the (Ac)Lys and Arg residues are D-amino acids. Even more particularly, the two (Ac)Lys and Arg residues are all D-amino acids.

In other embodiments, the amino acid sequence of the targeting peptidomimetic or B includes the formula X1-X2-Asn-Asn-X5-X6 (formula Ic), where X1 is (Ac)Lys or D-(Ac)Lys; X2 is Arg or D-Arg; X5 is Phe or D-Phe; and X6 is (Ac)Lys or D-(Ac)Lys; and where at least one (e.g., at least two, three, or four) of X1, X2, X5, or X6 is a D-amino acid.

In one embodiment of the amino acid sequence of formula I(c), two or more (e.g. three or more, four or more) amino acids are D-amino acids. In particular embodiments, more than one of X1, X2 and X6 are D-amino acids. Even more particularly, X1, X2 and X6 are all D-amino acids. In other embodiments, the amino acid sequence of the targeting peptidomimetic or B includes the formula X1-X2-Asn-Asn-X5-X6-X7 (formula Id), where X1 is (Ac)Lys or D-(Ac)Lys; X2 is Arg or D-Arg; X5 is Phe or D-Phe; X6 is (Ac)Lys or D-(Ac)Lys; and X7 is Tyr or D-Tyr; and where at least one (e.g., at least two, three, four, or five) of X1, X2, X5, X6, or X7 is a D-amino acid.

In one embodiment of the amino acid sequence of formula I(d), two or more (e.g. three or more, four or more) amino acids are D-amino acids. In particular embodiments, more than one of X1, X2 and X6 are D-amino acids. Even more particularly, X1, X2 and X6 are all D-amino acids.

In other embodiments, the amino acid sequence of the targeting peptidomimetic or B includes the formula Z1-X1-X2-Asn-Asn-X5-X6-X7-Z2 (formula Ie), where X1 is (Ac)Lys or D-(Ac)Lys; X2 is Arg or D-Arg; X5 is Phe or D-Phe; X6 is (Ac)Lys or D-(Ac)Lys; X7 is Tyr or D-Tyr; Z1 is absent, Cys, Gly, Cys-Gly, Arg-Gly, Cys-Arg-Gly, Ser-Arg-Gly, Cys-Ser-Arg-Gly, Gly-Ser-Arg-Gly, Cys-Gly-Ser-Arg-Gly, Gly-Gly-Ser-Arg-Gly, Cys-Gly-Gly-Ser-Arg-Gly, Tyr-Gly-Gly-Ser-Arg-Gly, Cys-Tyr-Gly-Gly-Ser-Arg-Gly, Phe-Tyr-Gly-Gly-Ser-Arg-Gly, Cys-Phe-Tyr-Gly-Gly-Ser-Arg-Gly, Phe-Phe-Tyr-Gly-Gly-Ser-Arg-Gly, Cys-Phe-Phe-Tyr-Gly-Gly-Ser-Arg-Gly, Thr-Phe-Phe-Tyr-Gly-Gly-Ser-Arg-Gly, or Cys-Thr-Phe-Phe-Tyr-Gly-Gly-Ser-Arg-Gly; and Z2 is absent, Cys, Tyr, Tyr-Cys, Cys-Tyr, Thr-Glu-Glu-Tyr, or Thr-Glu-Glu-Tyr-Cys; where at least one of X1, X2, X5, X6, or X7 is a D-amino acid; and where the polypeptide optionally includes one or more D-isomers of an amino acid recited in Z1 or Z2.

In one embodiment of the amino acid sequence of formula I(e), two or more (e.g. three or more, four or more) amino acids are D-amino acids. In particular embodiments, more than one of X1, X2 and X6 are D-amino acids. Even more particularly, X1, X2 and X6 are all D-amino acids. In certain embodiments, the amino acid sequence further includes one or more D-isomers of an amino acid recited in Z1.

In any of the above aspects, the amino acid sequence of the targeting peptidomimetic or B may be substantially identical to any of the sequences of Table 2, or a fragment thereof. In certain embodiments, the amino acid sequence has a sequence of Acetylated-Angiopep-1 (SEQ ID NO:175), Acetylated-Angiopep-2 (SEQ ID NO:200), Acetylated-Angiopep-3 (SEQ ID NO:209), Acetylated-Angiopep-4a (SEQ ID NO:210), Acetylated-Angiopep-4b (SEQ ID NO:211), Acetylated-Angiopep-5 (SEQ ID NO:212), Acetylated-Angiopep-6 (SEQ ID NO:213), Angiopep-7 (SEQ ID NO:112)) or reversed Acetylated-Angiopep-2 (SEQ ID NO:216). In certain embodiments, the amino acid sequence of the peptidomimetic or B has a sequence of SEQ ID NO:97 or SEQ ID No. 200 having from 0 to 5 (e.g., 0 to 4, 0 to 3, 0 to 2, 0 to 1, 1 to 5, 1 to 4, 1 to 3, 1 to 2, 2 to 5, 2 to 4, 2 to 3, 3 to 5, 3 to 4, or 4 to 5) substitutions, deletions, or additions of amino acids (e.g., a deletion of 1 to 5 amino acids from the C-terminus or the N-terminus).

In any of the above aspects, the amino acid sequence of the targeting peptidomimetic or B may have the amino acid sequence of Thr-Phe-Phe-Tyr-Gly-Gly-Ser-Arg-Gly-(Ac)Lys-Arg-Asn-Asn-Phe-(Ac)Lys-Thr-Glu-Glu-Tyr (An2), where any one or more amino acids are D-isomers. For example, the amino acid sequence can have 1, 2, 3, 4, or 5 amino acids which are D-isomers. In a preferred embodiment, one or more or all of positions 8, 10, and 11 can be D-isomers. In yet another embodiment, one or more or all of positions 8, 10, 11, and 15 can be D-isomers.

In any of the above aspects, the amino acid sequence of the targeting peptidomimetic or B may be Thr-Phe-Phe-Tyr-Gly-Gly-Ser-D-Arg-Gly-D-(Ac)Lys-D-Arg-Asn-Asn-Phe-D-(Ac)Lys-Thr-Glu-Glu-Tyr (4D-An2); Thr-Phe-Phe-Tyr-Gly-Gly-Ser-D-Arg-Gly-D-(Ac)Lys-D-Arg-Asn-Asn-Phe-(Ac)Lys-Thr-Glu-Glu-Tyr (3D-An2); Phe-Tyr-Gly-Gly-Ser-Arg-Gly-(Ac)Lys-Arg-Asn-Asn-Phe-(Ac)Lys-Thr-Glu-Glu-Tyr-Cys (P1); Phe-Tyr-Gly-Gly-Ser-Arg-Gly-D-(Ac)Lys-D-Arg-Asn-Asn-D-Phe-(Ac)Lys-Thr-Glu-Glu-Tyr-Cys (P1a); Phe-Tyr-Gly-Gly-Ser-Arg-Gly-D-(Ac)Lys-D-Arg-Asn-Asn-D-Phe-D-(Ac)Lys-Thr-Glu-Glu-Tyr-Cys (P1b); Phe-Tyr-Gly-Gly-Ser-Arg-Gly-D-(Ac)Lys-D-Arg-Asn-Asn-D-Phe-D-(Ac)Lys-Thr-Glu-Glu-D-Tyr-Cys (P1c); D-Phe-D-Tyr-Gly-Gly-Ser-D-Arg-Gly-D-(Ac)Lys-D-Arg-Asn-Asn-D-Phe-D-(Ac)Lys-Thr-Glu-D-Glu-D-Tyr-Cys (P1d); Gly-Gly-Ser-Arg-Gly-(Ac)Lys-Arg-Asn-Asn-Phe-(Ac)Lys-Thr-Glu-Glu-Tyr-Cys (P2); Ser-Arg-Gly-(Ac)Lys-Arg-Asn-Asn-Phe-(Ac)Lys-Thr-Glu-Glu-Tyr-Cys (P3); Gly-(Ac)Lys-Arg-Asn-Asn-Phe-(Ac)Lys-Thr-Glu-Glu-Tyr-Cys (P4); (Ac)Lys-Arg-Asn-Asn-Phe-(Ac)Lys-Thr-Glu-Glu-Tyr-Cys (P5); D-(Ac)Lys-D-Arg-Asn-Asn-D-Phe-(Ac)Lys-Thr-Glu-Glu-Tyr-Cys (P5a); D-(Ac)Lys-D-Arg-Asn-Asn-D-Phe-D-(Ac)Lys-Thr-Glu-Glu-Tyr-Cys (P5b); D-(Ac)Lys-D-Arg-Asn-Asn-D-Phe-D-(Ac)Lys-Thr-Glu-Glu-D-Tyr-Cys (P5c); (Ac)Lys-Arg-Asn-Asn-Phe-(Ac)Lys-Tyr-Cys (P6); D-(Ac)Lys-D-Arg-Asn-Asn-D-Phe-(Ac)Lys-Tyr-Cys (P6a); D-(Ac)Lys-D-Arg-Asn-Asn-D-Phe-D-(Ac)Lys-Tyr-Cys (P6b); Thr-Phe-Phe-Tyr-Gly-Gly-Ser-D-Arg-Gly-D-(Ac)Lys-D-Arg-Asn-Asn-Phe-D-(Ac)Lys-Thr-Glu-Glu-Tyr; and D-(Ac)Lys-D-Arg-Asn-Asn-D-Phe-D-(Ac)Lys-D-Tyr-Cys (P6c); or a fragment thereof. In other embodiments, the amino acid sequence of the targeting peptidomimetic or B has a sequence of one of the aforementioned peptides having from 0 to 5 (e.g., from 0 to 4, 0 to 3, 0 to 2, 0 to 1, 1 to 5, 1 to 4, 1 to 3, 1 to 2, 2 to 5, 2 to 4, 2 to 3, 3 to 5, 3 to 4, or 4 to 5) substitutions, deletions, or additions of amino acids.

In any of the above aspects the amino acid sequence of the targeting peptidomimetic or B may be Phe-Tyr-Gly-Gly-Ser-Arg-Gly-(Ac)Lys-Arg-Asn-Asn-Phe-(Ac)Lys-Thr-Glu-Glu; Gly-Gly-Ser-Arg-Gly-(Ac)Lys-Arg-Asn-Asn-Phe-(Ac)Lys-Thr-Glu-Glu; Ser-Arg-Gly-(Ac)Lys-Arg-Asn-Asn-Phe-(Ac)Lys-Thr-Glu-Glu; Gly-(Ac)Lys-Arg-Asn-Asn-Phe-(Ac)Lys-Thr-Glu-Glu; (Ac)Lys-Arg-Asn-Asn-Phe-(Ac)Lys-Thr-Glu-Glu; or (Ac)Lys-Arg-Asn-Asn-Phe-(Ac)Lys, or a fragment thereof.

In any of the above aspects, the amino acid sequence of the targeting peptidomimetic or B may be Thr-Phe-Phe-Tyr-Gly-Gly-Ser-D-Arg-Gly-D-(Ac)Lys-D-Arg-Asn-Asn-Phe-D-(Ac)Lys-Thr-Glu-Glu-Tyr (4D-An2); Thr-Phe-Phe-Tyr-Gly-Gly-Ser-D-Arg-Gly-D-(Ac)Lys-D-Arg-Asn-Asn-Phe-(Ac)Lys-Thr-Glu-Glu-Tyr (3D-An2); Phe-Tyr-Gly-Gly-Ser-Arg-Gly-(Ac)Lys-Arg-Asn-Asn-Phe-(Ac)Lys-Thr-Glu-Glu-Tyr-Cys (P1); Phe-Tyr-Gly-Gly-Ser-Arg-Gly-D-(Ac)Lys-D-Arg-Asn-Asn-D-Phe-(Ac)Lys-Thr-Glu-Glu-Tyr-Cys (P1a); Phe-Tyr-Gly-Gly-Ser-Arg-Gly-D-(Ac)Lys-D-Arg-Asn-Asn-D-Phe-D-(Ac)Lys-Thr-Glu-Glu-Tyr-Cys (P1b); Phe-Tyr-Gly-Gly-Ser-Arg-Gly-D-(Ac)Lys-D-Arg-Asn-Asn-D-Phe-D-(Ac)Lys-Thr-Glu-Glu-D-Tyr-Cys (P1c); D-Phe-D-Tyr-Gly-Gly-Ser-D-Arg-Gly-D-(Ac)Lys-D-Arg-Asn-Asn-D-Phe-D-(Ac)Lys-Thr-Glu-D-Glu-D-Tyr-Cys (P1d) or a fragment thereof (e.g., deletion of 1 to 7 amino acids from the N-terminus of P1, P1a, P1b, P1c, or P1d; a deletion of 1 to 5 amino acids from the C-terminus of P1, P1a, P1b, P1c, or P1d; or deletions of 1 to 7 amino acids from the N-terminus of P1, P1a, P1b, P1c, or P1d and 1 to 5 amino acids from the C-terminus of P1, P1a, P1b, P1c, or P1d).

TABLE 2
Exemplary polypeptides

| SEQ | | | | | | | | | | | | | | | | | | | |
|---|---|---|---|---|---|---|---|---|---|---|---|---|---|---|---|---|---|---|---|
| ID NO: | | | | | | | | | | | | | | | | | | | |
| 1 | T | F | V | Y | G | G | C | R | A | NεpK | R | N | N | F | NεpK | S | A | E | D |
| 2 | T | F | Q | Y | G | G | C | M | G | N | G | N | N | F | V | T | E | NεpK | E |
| 3 | P | F | F | Y | G | G | C | G | G | N | R | N | N | F | D | T | E | E | Y |
| 4 | S | F | Y | Y | G | G | C | L | G | N | NεpK | N | N | Y | L | R | E | E | E |
| 5 | T | F | F | Y | G | G | C | R | A | NεpK | R | N | N | F | NεpK | R | A | NεpK | Y |
| 6 | T | F | F | Y | G | G | C | R | G | NεpK | R | N | N | F | NεpK | R | A | NεpK | Y |
| 7 | T | F | F | Y | G | G | C | R | A | NεpK | NεpK | N | N | Y | NεpK | R | A | NεpK | Y |
| 8 | T | F | F | Y | G | G | C | R | G | NεpK | NεpK | N | N | F | NεpK | R | A | NεpK | Y |
| 9 | T | F | Q | Y | G | G | C | R | A | NεpK | R | N | N | F | NεpK | R | A | NεpK | Y |
| 10 | T | F | Q | Y | G | G | C | R | G | NεpK | NεpK | N | N | F | NεpK | R | A | NεpK | Y |
| 11 | T | F | F | Y | G | G | C | L | G | NεpK | R | N | N | F | NεpK | R | A | NεpK | Y |
| 12 | T | F | F | Y | G | G | S | L | G | NεpK | R | N | N | F | NεpK | R | A | NεpK | Y |
| 13 | P | F | F | Y | G | G | C | G | G | NεpK | NεpK | N | N | F | NεpK | R | A | NεpK | Y |
| 14 | T | F | F | Y | G | G | C | R | G | NεpK | G | N | N | Y | NεpK | R | A | NεpK | Y |
| 15 | P | F | F | Y | G | G | C | R | G | NεpK | R | N | N | F | L | R | A | NεpK | Y |
| 16 | T | F | F | Y | G | G | C | R | G | NεpK | R | N | N | F | NεpK | R | E | NεpK | Y |
| 17 | P | F | F | Y | G | G | C | R | A | NεpK | NεpK | N | N | F | NεpK | R | A | NεpK | E |
| 18 | T | F | F | Y | G | G | C | R | G | NεpK | R | N | N | F | NεpK | R | A | NεpK | D |
| 19 | T | F | F | Y | G | G | C | R | A | NεpK | R | N | N | F | D | R | A | NεpK | Y |
| 20 | T | F | F | Y | G | G | C | R | G | NεpK | NεpK | N | N | F | NεpK | R | A | E | Y |
| 21 | P | F | F | Y | G | G | C | G | A | N | R | N | N | F | NεpK | R | A | NεpK | Y |
| 22 | T | F | F | Y | G | G | C | G | G | NεpK | NεpK | N | N | F | NεpK | T | A | NεpK | Y |
| 23 | T | F | F | Y | G | G | C | R | G | N | R | N | N | F | L | R | A | NεpK | Y |
| 24 | T | F | F | Y | G | G | C | R | G | N | R | N | N | F | NεpK | T | A | NεpK | Y |
| 25 | T | F | F | Y | G | G | S | R | G | N | R | N | N | F | NεpK | T | A | NεpK | Y |
| 26 | T | F | F | Y | G | G | C | L | G | N | G | N | N | F | NεpK | R | A | NεpK | Y |
| 27 | T | F | F | Y | G | G | C | L | G | N | R | N | N | F | L | R | A | NεpK | Y |
| 28 | T | F | F | Y | G | G | C | L | G | N | R | N | N | F | NεpK | T | A | NεpK | Y |
| 29 | T | F | F | Y | G | G | C | R | G | N | G | N | N | F | NεpK | S | A | NεpK | Y |
| 30 | T | F | F | Y | G | G | C | R | G | NεpK | NεpK | N | N | F | D | R | E | NεpK | Y |
| 31 | T | F | F | Y | G | G | C | R | G | NεpK | R | N | N | F | L | R | E | NεpK | E |
| 32 | T | F | F | Y | G | G | C | R | G | NεpK | G | N | N | F | D | R | A | NεpK | Y |
| 33 | T | F | F | Y | G | G | S | R | G | NεpK | G | N | N | F | D | R | A | NεpK | Y |
| 34 | T | F | F | Y | G | G | C | R | G | N | G | N | N | F | V | T | A | NεpK | Y |
| 35 | P | F | F | Y | G | G | C | G | G | NεpK | G | N | N | Y | V | T | A | NεpK | Y |
| 36 | T | F | F | Y | G | G | C | L | G | NεpK | G | N | N | F | L | T | A | NεpK | Y |
| 37 | S | F | F | Y | G | G | C | L | G | N | NεpK | N | N | F | L | T | A | NεpK | Y |
| 38 | T | F | F | Y | G | G | C | G | G | N | NεpK | N | N | F | V | R | E | NεpK | Y |
| 39 | T | F | F | Y | G | G | C | M | G | N | NεpK | N | N | F | V | R | E | NεpK | Y |
| 40 | T | F | F | Y | G | G | S | M | G | N | NεpK | N | N | F | V | R | E | NεpK | Y |
| 41 | P | F | F | Y | G | G | C | L | G | N | R | N | N | Y | V | R | E | NεpK | Y |
| 42 | T | F | F | Y | G | G | C | L | G | N | R | N | N | F | V | R | E | NεpK | Y |
| 43 | T | F | F | Y | G | G | C | L | G | N | NεpK | N | N | Y | V | R | E | NεpK | Y |
| 44 | T | F | F | Y | G | G | C | G | G | N | G | N | N | F | L | T | A | NεpK | Y |
| 45 | T | F | F | Y | G | G | C | R | G | N | R | N | N | F | L | T | A | E | Y |
| 46 | T | F | F | Y | G | G | C | R | G | N | G | N | N | F | NεpK | S | A | E | Y |
| 47 | P | F | F | Y | G | G | C | L | G | N | NεpK | N | N | F | NεpK | T | A | E | Y |
| 48 | T | F | F | Y | G | G | C | R | G | N | R | N | N | F | NεpK | T | E | E | Y |
| 49 | T | F | F | Y | G | G | C | R | G | NεpK | R | N | N | F | NεpK | T | E | E | D |
| 50 | P | F | F | Y | G | G | C | G | G | N | G | N | N | F | V | R | E | NεpK | Y |
| 51 | S | F | F | Y | G | G | C | M | G | N | G | N | N | F | V | R | E | NεpK | Y |
| 52 | P | F | F | Y | G | G | C | G | G | N | G | N | N | F | L | R | E | NεpK | Y |
| 53 | T | F | F | Y | G | G | C | L | G | N | G | N | N | F | V | R | E | NεpK | Y |
| 54 | S | F | F | Y | G | G | C | L | G | N | G | N | N | Y | L | R | E | NεpK | Y |
| 55 | T | F | F | Y | G | G | S | L | G | N | G | N | N | F | V | R | E | NεpK | Y |
| 56 | T | F | F | Y | G | G | C | R | G | N | G | N | N | F | V | T | A | E | Y |
| 57 | T | F | F | Y | G | G | C | L | G | NεpK | G | N | N | F | V | S | A | E | Y |
| 58 | T | F | F | Y | G | G | C | L | G | N | R | N | N | F | D | R | A | E | Y |
| 59 | T | F | F | Y | G | G | C | L | G | N | R | N | N | F | L | R | E | E | Y |
| 60 | T | F | F | Y | G | G | C | L | G | N | NεpK | N | N | Y | L | R | E | E | Y |
| 61 | P | F | F | Y | G | G | C | G | G | N | R | N | N | Y | L | R | E | E | Y |
| 62 | P | F | F | Y | G | G | S | G | G | N | R | N | N | Y | L | R | E | E | Y |
| 63 | M | R | P | D | F | C | L | E | P | P | Y | T | G | P | C | V | A | R | I |
| 64 | A | R | I | I | R | Y | F | Y | N | A | NεpK | A | G | L | C | Q | T | F | V |
| | Y | G | | | | | | | | | | | | | | | | | |
| 65 | Y | G | G | C | R | A | NεpK | R | N | N | Y | NεpK | S | A | E | D | C | M | R |
| | T | C | G | | | | | | | | | | | | | | | | |
| 66 | P | D | F | C | L | E | P | P | Y | T | G | P | C | V | A | R | I | I | R |
| | Y | F | Y | | | | | | | | | | | | | | | | |
| 67 | T | F | F | Y | G | G | C | R | G | NεpK | R | N | N | F | NεpK | T | E | E | Y |
| 68 | NεpK | F | F | Y | G | G | C | R | G | NεpK | R | N | N | F | NεpK | T | E | E | Y |
| 69 | T | F | Y | Y | G | G | C | R | G | NεpK | R | N | N | Y | NεpK | T | E | E | Y |
| 71 | C | T | F | F | Y | G | C | C | R | G | NεpK | R | N | N | F | NεpK | T | E | E |
| | Y | | | | | | | | | | | | | | | | | | |
| 72 | T | F | F | Y | G | G | C | R | G | NεpK | R | N | N | F | NεpK | T | E | E | Y |
| | C | | | | | | | | | | | | | | | | | | |
| 73 | C | T | F | F | Y | G | S | C | R | G | NεpK | R | N | N | F | NεpK | T | E | E |
| | Y | | | | | | | | | | | | | | | | | | |
| 75 | P | F | F | Y | G | G | C | R | G | NεpK | R | N | N | F | NεpK | T | E | E | Y |
| 76 | T | F | F | Y | G | G | C | R | G | NεpK | R | N | N | F | NεpK | T | NεpK | E | Y |
| 77 | T | F | F | Y | G | G | NεpK | R | G | NεpK | R | N | N | F | NεpK | T | E | E | Y |
| 78 | T | F | F | Y | G | G | C | R | G | NεpK | R | N | N | F | NεpK | T | NεpK | R | Y |
| 79 | T | F | F | Y | G | G | NεpK | R | G | NεpK | R | N | N | F | NεpK | T | A | E | Y |
| 80 | T | F | F | Y | G | G | NεpK | R | G | NεpK | R | N | N | F | NεpK | T | A | G | Y |
| 81 | T | F | F | Y | G | G | NεpK | R | G | NεpK | R | N | N | F | NεpK | R | E | NεpK | Y |
| 82 | T | F | F | Y | G | G | NεpK | R | G | NεpK | R | N | N | F | NεpK | R | A | NεpK | Y |
| 83 | T | F | F | Y | G | G | C | L | G | N | R | N | N | F | NεpK | T | E | E | Y |
| 84 | T | F | F | Y | G | C | G | R | G | NεpK | R | N | N | F | NεpK | T | E | E | Y |
| 85 | T | F | F | Y | G | G | R | C | G | NεpK | R | N | N | F | NεpK | T | E | E | Y |
| 86 | T | F | F | Y | G | G | C | L | G | N | G | N | N | F | D | T | E | E | E |
| 87 | T | F | Q | Y | G | G | C | R | G | NεpK | R | N | N | F | NεpK | T | E | E | Y |
| 88 | Y | N | NεpK | E | F | G | T | F | N | T | NεpK | G | C | E | R | G | Y | R | F |
| 89 | R | F | NεpK | Y | G | G | C | L | G | N | M | N | N | F | E | T | L | E | E |
| 90 | R | F | NεpK | Y | G | G | C | L | G | N | NεpK | N | N | F | L | R | L | NεpK | Y |
| 91 | R | F | NεpK | Y | G | G | C | L | G | N | NεpK | N | N | Y | L | R | L | NεpK | Y |
| 92 | NεpK | T | NεpK | R | NεpK | R | NεpK | NεpK | Q | R | V | NεpK | I | A | Y | E | E | I | F |
| | NεpK | N | Y | | | | | | | | | | | | | | | | |
| 93 | NεpK | T | NεpK | R | NεpK | R | NεpK | NεpK | Q | R | V | NεpK | I | A | Y | | | | |
| 94 | R | G | G | R | L | S | Y | S | R | R | F | S | T | S | T | G | R | | |
| 95 | R | R | L | S | Y | S | R | R | R | F | | | | | | | | | |
| 96 | R | Q | I | NεpK | I | W | F | Q | N | R | R | M | NεpK | W | NεpK | NεpK | | | |
| 97 | T | F | F | Y | G | G | S | R | G | NεpK | R | N | N | F | NεpK | T | E | E | Y |
| 98 | M | R | P | D | F | C | L | E | P | P | Y | T | G | P | C | V | A | R | I |
| | I | R | Y | F | Y | N | A | NεpK | A | G | L | C | Q | T | F | V | Y | G | G |
| | C | R | A | NεpK | R | N | N | F | NεpK | S | A | E | D | C | M | R | T | C | G |
| | G | A | | | | | | | | | | | | | | | | | |
| 99 | T | F | F | Y | G | G | C | R | G | NεpK | R | N | N | F | NεpK | T | NεpK | E | Y |
| 100 | R | F | NεpK | Y | G | G | C | L | G | N | NεpK | N | N | Y | L | R | L | NεpK | Y |
| 101 | T | F | F | Y | G | G | C | R | A | NεpK | R | N | N | F | NεpK | R | A | NεpK | Y |
| 102 | N | A | NεpK | A | G | L | C | Q | T | F | V | Y | G | G | C | L | A | NεpK | R |
| | N | N | F | E | S | A | E | D | C | M | R | T | C | G | G | A | | | |
| 103 | Y | G | G | C | R | A | NεpK | R | N | N | F | NεpK | S | A | E | D | C | M | R |
| | T | C | G | G | A | | | | | | | | | | | | | | |
| 104 | G | L | C | Q | T | F | V | Y | G | G | C | R | A | NεpK | R | N | N | F | NεpK |
| | S | A | E | | | | | | | | | | | | | | | | |
| 105 | L | C | Q | T | F | V | Y | G | G | C | E | A | NεpK | R | N | N | F | NεpK | S |
| | A | | | | | | | | | | | | | | | | | | |
| 107 | T | F | F | Y | G | G | S | R | G | NεpK | R | N | N | F | NεpK | T | E | E | Y |
| 108 | R | F | F | Y | G | G | S | R | G | NεpK | R | N | N | F | NεpK | T | E | E | Y |
| 109 | R | F | F | Y | G | G | S | R | G | NεpK | R | N | N | F | NεpK | T | E | E | Y |
| 110 | R | F | F | Y | G | G | S | R | G | NεpK | R | N | N | F | R | T | E | E | Y |
| 111 | T | F | F | Y | G | G | S | R | G | NεpK | R | N | N | F | R | T | E | E | Y |
| 112 | T | F | F | Y | G | G | S | R | G | R | R | N | N | F | R | T | E | E | Y |
| 113 | C | T | F | F | Y | G | G | S | R | G | NεpK | R | N | N | F | NεpK | T | E | E |
| | Y | I | | | | | | | | | | | | | | | | | |
| 114 | T | F | F | Y | G | G | S | R | G | NεpK | R | N | N | F | NεpK | T | E | E | Y |
| | C | | | | | | | | | | | | | | | | | | |
| 115 | C | T | F | F | Y | G | G | S | R | G | R | R | N | N | F | R | T | E | E |
| | Y | | | | | | | | | | | | | | | | | | |
| 116 | T | F | F | Y | G | G | S | R | G | R | R | N | N | F | R | T | E | E | Y |
| | C | | | | | | | | | | | | | | | | | | |
| 117 | Y | E | E | T | NεpK | F | N | N | R | NεpK | G | R | S | G | G | Y | F | F | T |
| 118 | T | F | V | Y | G | G | C | R | A | AcK | R | N | N | F | AcK | S | A | E | D |
| 119 | T | F | Q | Y | G | G | C | M | G | N | G | N | N | F | V | T | E | AcK | E |
| 120 | S | F | Y | Y | G | G | C | L | G | N | AcK | N | N | Y | L | R | E | E | E |
| 121 | T | F | F | Y | G | G | C | R | A | AcK | R | N | N | F | AcK | R | A | AcK | Y |
| 122 | T | F | F | Y | G | G | C | R | G | AcK | R | N | N | F | AcK | R | A | AcK | Y |
| 123 | T | F | F | Y | G | G | C | R | A | AcK | AcK | N | N | Y | AcK | R | A | AcK | Y |
| 124 | T | F | F | Y | G | G | C | R | G | AcK | AcK | N | N | F | AcK | R | A | AcK | Y |
| 125 | T | F | Q | Y | G | G | C | R | A | AcK | R | N | N | F | AcK | R | A | AcK | Y |
| 126 | T | F | Q | Y | G | G | C | R | G | AcK | AcK | N | N | F | AcK | R | A | AcK | Y |
| 127 | T | F | F | Y | G | G | C | L | G | AcK | R | N | N | F | AcK | R | A | AcK | Y |
| 128 | T | F | F | Y | G | G | S | L | G | AcK | R | N | N | F | AcK | R | A | AcK | Y |
| 129 | P | F | F | Y | G | G | C | G | G | AcK | AcK | N | N | F | AcK | R | A | AcK | Y |
| 130 | T | F | F | Y | G | G | C | R | G | AcK | G | N | N | Y | AcK | R | A | AcK | Y |
| 131 | P | F | F | Y | G | G | C | R | G | AcK | R | N | N | F | L | R | A | AcK | Y |
| 132 | T | F | F | Y | G | G | C | R | G | AcK | R | N | N | F | AcK | R | E | AcK | Y |
| 133 | P | F | F | Y | G | G | C | R | A | AcK | AcK | N | N | F | AcK | R | A | AcK | E |
| 134 | T | F | F | Y | G | G | C | R | G | AcK | R | N | N | F | AcK | R | A | AcK | D |
| 135 | T | F | F | Y | G | G | C | R | A | AcK | R | N | N | F | D | R | A | AcK | Y |
| 136 | T | F | F | Y | G | G | C | R | G | AcK | AcK | N | N | F | AcK | R | A | E | Y |
| 137 | P | F | F | Y | G | G | C | G | A | N | R | N | N | F | AcK | R | A | AcK | Y |
| 138 | T | F | F | Y | G | G | C | G | G | AcK | AcK | N | N | F | AcK | T | A | AcK | Y |
| 139 | T | F | F | Y | G | G | C | R | G | N | R | N | N | F | L | R | A | AcK | Y |
| 140 | T | F | F | Y | G | G | C | R | G | N | R | N | N | F | AcK | T | A | AcK | Y |
| 141 | T | F | F | Y | G | G | S | R | G | N | R | N | N | F | AcK | T | A | AcK | Y |
| 142 | T | F | F | Y | G | G | C | L | G | N | G | N | N | F | AcK | R | A | AcK | Y |
| 143 | T | F | F | Y | G | G | C | L | G | N | R | N | N | F | L | R | A | AcK | Y |
| 144 | T | F | F | Y | G | G | C | L | G | N | R | N | N | F | AcK | T | A | AcK | Y |
| 145 | T | F | F | Y | G | G | C | R | G | N | G | N | N | F | AcK | S | A | AcK | Y |
| 146 | T | F | F | Y | G | G | C | R | G | AcK | AcK | N | N | F | D | R | E | AcK | Y |
| 147 | T | F | F | Y | G | G | C | R | G | AcK | R | N | N | F | L | R | E | AcK | E |
| 148 | T | F | F | Y | G | G | C | R | G | AcK | G | N | N | F | D | R | A | AcK | Y |
| 149 | T | F | F | Y | G | G | S | R | G | AcK | G | N | N | F | D | R | A | AcK | Y |
| 150 | T | F | F | Y | G | G | C | R | G | N | G | N | N | F | V | T | A | AcK | Y |
| 151 | P | F | F | Y | G | G | C | G | G | AcK | G | N | N | Y | V | T | A | AcK | Y |
| 152 | T | F | F | Y | G | G | C | L | G | AcK | G | N | N | F | L | T | A | AcK | Y |
| 153 | S | F | F | Y | G | G | C | L | G | N | AcK | N | N | F | L | T | A | AcK | Y |
| 154 | T | F | F | Y | G | G | C | G | G | N | AcK | N | N | F | V | R | E | AcK | Y |
| 155 | T | F | F | Y | G | G | C | M | G | N | AcK | N | N | F | V | R | E | AcK | Y |
| 156 | T | F | F | Y | G | G | S | M | G | N | AcK | N | N | F | V | R | E | AcK | Y |
| 157 | P | F | F | Y | G | G | C | L | G | N | R | N | N | Y | V | R | E | AcK | Y |
| 158 | T | F | F | Y | G | G | C | L | G | N | R | N | N | F | V | R | E | AcK | Y |
| 159 | T | F | F | Y | G | G | C | L | G | N | AcK | N | N | Y | V | R | E | AcK | Y |
| 160 | T | F | F | Y | G | G | C | G | G | N | G | N | N | F | L | T | A | AcK | Y |
| 161 | T | F | F | Y | G | G | C | R | G | N | G | N | N | F | AcK | S | A | E | Y |
| 162 | P | F | F | Y | G | G | C | L | G | N | AcK | N | N | F | AcK | T | A | E | Y |
| 163 | T | F | F | Y | G | G | C | R | G | N | R | N | N | F | AcK | T | E | E | Y |
| 164 | T | F | F | Y | G | G | C | R | G | AcK | R | N | N | F | AcK | T | E | E | D |
| 165 | P | F | F | Y | G | G | C | G | G | N | G | N | N | F | V | R | E | AcK | Y |
| 166 | S | F | F | Y | G | G | C | M | G | N | G | N | N | F | V | R | E | AcK | Y |
| 167 | P | F | F | Y | G | G | C | G | G | N | G | N | N | F | L | R | E | AcK | Y |
| 168 | T | F | F | Y | G | G | C | L | G | N | G | N | N | F | V | R | E | AcK | Y |
| 169 | S | F | F | Y | G | G | C | L | G | N | G | N | N | Y | L | R | E | AcK | Y |
| 170 | T | F | F | Y | G | G | S | L | G | N | G | N | N | F | V | R | E | AcK | Y |
| 171 | T | F | F | Y | G | G | C | L | G | AcK | G | N | N | F | V | S | A | E | Y |
| 172 | T | F | F | Y | G | G | C | L | G | N | AcK | N | N | Y | L | R | E | E | Y |
| 173 | A | R | I | I | R | Y | F | Y | N | A | AcK | A | G | L | C | Q | T | F | V |
| | Y | G | | | | | | | | | | | | | | | | | |
| 174 | Y | G | G | C | R | A | AcK | R | N | N | Y | AcK | S | A | E | D | C | M | R |
| | T | C | G | | | | | | | | | | | | | | | | |
| 175 | T | F | F | Y | G | G | C | R | G | AcK | R | N | N | F | AcK | T | E | E | Y |
| 176 | AcK | F | F | Y | G | G | C | R | G | AcK | R | N | N | F | AcK | T | E | E | Y |
| 177 | T | F | Y | Y | G | G | C | R | G | AcK | R | N | N | Y | AcK | T | E | E | Y |
| 178 | C | T | F | F | Y | G | C | C | R | G | AcK | R | N | N | F | AcK | T | E | E |
| | Y | | | | | | | | | | | | | | | | | | |
| 179 | T | F | F | Y | G | G | C | R | G | AcK | R | N | N | F | AcK | T | E | E | Y |
| | C | | | | | | | | | | | | | | | | | | |
| 180 | C | T | F | F | Y | G | S | C | R | G | AcK | R | N | N | F | AcK | T | E | E |
| | Y | | | | | | | | | | | | | | | | | | |
| 181 | P | F | F | Y | G | G | C | R | G | AcK | R | N | N | F | AcK | T | E | E | Y |
| 182 | T | F | F | Y | G | G | C | R | G | AcK | R | N | N | F | AcK | T | AcK | E | Y |
| 183 | T | F | F | Y | G | G | AcK | R | G | AcK | R | N | N | F | AcK | T | E | E | Y |
| 184 | T | F | F | Y | G | G | C | R | G | AcK | R | N | N | F | AcK | T | AcK | R | Y |
| 185 | T | F | F | Y | G | G | AcK | R | G | AcK | R | N | N | F | AcK | T | A | E | Y |
| 186 | T | F | F | Y | G | G | AcK | R | G | AcK | R | N | N | F | AcK | T | A | G | Y |
| 187 | T | F | F | Y | G | G | AcK | R | G | AcK | R | N | N | F | AcK | R | E | AcK | Y |
| 188 | T | F | F | Y | G | G | AcK | R | G | AcK | R | N | N | F | AcK | R | A | AcK | Y |
| 189 | T | F | F | Y | G | G | C | L | G | N | R | N | N | F | AcK | T | E | E | Y |
| 190 | T | F | F | Y | G | C | G | R | G | AcK | R | N | N | F | AcK | T | E | E | Y |
| 191 | T | F | F | Y | G | G | R | C | G | AcK | R | N | N | F | AcK | T | E | E | Y |
| 192 | T | F | Q | Y | G | G | C | R | G | AcK | R | N | N | F | AcK | T | E | E | Y |
| 193 | Y | N | AcK | E | F | G | T | F | N | T | AcK | G | C | E | R | G | Y | R | F |
| 194 | R | F | AcK | Y | G | G | C | L | G | N | M | N | N | F | E | T | L | E | E |
| 195 | R | F | AcK | Y | G | G | C | L | G | N | AcK | N | N | F | L | R | L | AcK | Y |
| 196 | R | F | AcK | Y | G | G | C | L | G | N | AcK | N | N | Y | L | R | L | AcK | Y |
| 197 | AcK | T | AcK | R | AcK | R | AcK | AcK | Q | R | V | AcK | I | A | Y | E | E | I | F |
| | AcK | N | Y | | | | | | | | | | | | | | | | |
| 198 | AcK | T | AcK | R | AcK | R | AcK | AcK | Q | R | V | AcK | I | A | Y | | | | |
| 199 | R | Q | I | AcK | I | W | F | Q | N | R | R | M | AcK | W | AcK | AcK | | | |
| 200 | T | F | F | Y | G | G | S | R | G | AcK | R | N | N | F | AcK | T | E | E | Y |
| 201 | M | R | P | D | F | C | L | E | P | P | Y | T | G | P | C | V | A | R | I |
| | I | R | Y | F | Y | N | A | AcK | A | G | L | C | Q | T | F | V | Y | G | G |
| | C | R | A | AcK | R | N | N | F | AcK | S | A | E | D | C | M | R | T | C | G |
| | G | A | | | | | | | | | | | | | | | | | |
| 202 | T | G | G | Y | G | G | C | R | G | AcK | R | N | N | F | AcK | T | AcK | E | Y |
| 203 | R | F | AcK | Y | G | G | C | L | G | N | AcK | N | N | Y | L | R | L | AcK | Y |
| 204 | T | F | F | Y | G | G | C | R | A | AcK | R | N | N | F | AcK | R | A | AcK | Y |
| 205 | N | A | AcK | A | G | L | C | Q | T | F | V | Y | G | G | C | L | A | AcK | R |
| | N | N | F | E | S | A | E | D | C | M | R | T | C | G | G | A | | | |
| 206 | Y | G | G | C | R | A | AcK | R | N | N | F | AcK | S | A | E | D | C | M | R |
| | T | C | G | G | A | | | | | | | | | | | | | | |
| 207 | G | L | C | Q | T | F | V | Y | G | G | C | R | A | AcK | R | N | N | F | AcK |
| | S | A | E | | | | | | | | | | | | | | | | |
| 208 | L | C | Q | T | F | V | Y | G | G | C | E | A | AcK | R | N | N | F | AcK | S |
| | A | | | | | | | | | | | | | | | | | | |
| 209 | T | F | F | Y | G | G | S | R | G | AcK | R | N | N | F | AcK | T | E | E | Y |
| 210 | R | F | F | Y | G | G | S | R | G | AcK | R | N | N | F | AcK | T | E | E | Y |
| 211 | R | F | F | Y | G | G | S | R | G | AcK | R | N | N | F | AcK | T | E | E | Y |
| 212 | R | F | F | Y | G | G | S | R | G | AcK | R | N | N | F | R | T | E | E | Y |
| 213 | T | F | F | Y | G | G | S | R | G | AcK | R | N | N | F | R | T | E | E | Y |
| 214 | C | T | F | F | Y | G | G | S | R | G | AcK | R | N | N | F | AcK | T | E | E |
| | Y | | | | | | | | | | | | | | | | | | |
| 215 | T | F | F | Y | G | G | S | R | G | AcK | R | N | N | F | AcK | T | E | E | Y |
| | C | | | | | | | | | | | | | | | | | | |
| 216 | Y | E | E | T | AcK | F | N | N | R | AcK | G | R | S | G | G | Y | F | F | T |

NεpK = Nε-protected-L-lysine
AcK = Nε-(acetyl)-L-lysine
Polypeptides Nos. 5, 67, 76, 91, 121, 175, 182 and 196, include the sequences of SEQ ID NOS: 5, 67, 76, 91, 121, 175, 182 and 196 respectively, and are amidated at the C-terminus.
Polypeptides Nos. 107, 109, 110, 200, 211 and 212 include the sequences of SEQ ID NOS: 97, 109, 110, 200, 211 and 212 respectively, and are acetylated at the N-terminus.

In certain embodiments, the amino acid sequence of the targeting peptidomimetic or B may have at least 70%, 75%, 80%, 85%, 90%, 95%, 99%, or even 100% identity to a sequence described herein. The amino acid sequence of the targeting peptidomimetic or B may differ in having one or more (e.g., 2, 3, 4, 5, 6, 7, 8, 9, 10, 11, 12, 13, 14, or 15) additions, substitutions or deletions relative to a sequence described herein. For example, the amino acid sequence may be a fragment of an amino acid sequence that retains targeting activity (such that the fragments are capable of efficiently being transported to or accumulating in a particular cell type (e.g., liver, eye, lung, kidney, or spleen) and/or are efficiently transported across the BBB, i.e., a functional fragment). In certain embodiments, the amino acid sequence of the targeting peptidomimetic or B may differ in having 1, 2, 3, 4, 5, 6, 7, 8, 9, 10, 11, 12, or more amino acids deleted from either the N-terminus, the C-terminus, or a combination thereof. Other fragments include sequences where internal portions of the amino acid sequence of the targeting peptidomimetic or B are deleted. In particular embodiments, the targeting peptidomimetic or B differs from a sequence described herein by the deletion of 1, 2, 3, 4, or 5 amino acids (e.g., 1 to 3 amino acids).

In alternative embodiments, the amino acid sequence of the targeting peptidomimetic or B may have one or more (e.g., 2, 3, 4, 5, 6, 7, 8, 9, 10, 11, 12, 13, 14, or 15) substitutions relative to a sequence described herein. More particularly, the targeting peptidomimetic or B differs from a sequence described herein by the deletion of 1, 2, 3, 4, or 5 amino acids (e.g., 1 to 3 amino acids).

In yet further embodiments, the amino acid sequence of the targeting peptidomimetic or B may have one or more (e.g., 2, 3, 4, 5, 6, 7, 8, 9, 10, 11, 12, 13, 14, or 15) additions relative to a sequence described herein. More particularly, the targeting peptidomimetic or B differs from a sequence described herein by the deletion of 1, 2, 3, 4, or 5 amino acids (e.g., 1 to 3 amino acids).

In some embodiments, the amino acid sequence of the targeting peptidomimetic or B has one or more additional cysteine residues N- or C-terminal of the amino acid sequence, or both. In other embodiments, the amino acid sequence of the targeting peptidomimetic or B may have one or more additional tyrosine residues N- or C-terminal of the amino acid sequence, or both. In yet further embodiments, the amino acid sequence of the targeting peptidomimetic or B has the amino acid sequence Tyr-Cys and/or Cys-Tyr N- or C-terminal of the amino acid sequence, or both.

The amino acid sequence of the targeting peptidomimetic or B may also contain other modifications. These are described in greater detail below in the section entitled Polypeptide derivatives and peptidomimetics.

The amino acid sequence of the targeting peptidomimetic or B may be less than 30, 25, 24, 23, 22, 21, 20, or 19 amino acids in length. The amino acid sequence may be produced by recombinant genetic technology or chemical synthesis.

The amino acid sequences described in Table 2 have different targeting properties. Certain sequences may be efficiently transported into a particular cell type (e.g., any one, two, three, four, or five of liver, lung, kidney, spleen, and muscle) or may cross the mammalian BBB efficiently (e.g., Acetylated-Angiopep-1, -2, -3, -4a, -4b, -5, and -6). Other sequences are able to enter a particular cell type (e.g., any one, two, three, four, or five of liver, lung, kidney, spleen, and muscle) but do not cross the BBB efficiently (e.g., a conjugate including Angiopep-7).

Additional amino acid sequences of targeting peptidomimetics (B) having particular targeting properties may be identified by using one of the assays or methods described herein. For example, a targeting peptidomimetic or B capable of crossing the mammalian BBB efficiently may be identified based on its location in the parenchyma in an in situ cerebral perfusion assay.

Assays to determine accumulation in other tissues may be performed as well. Labelled conjugates of a targeting peptidomimetic can be administered to an animal, and accumulation in different organs can be measured. For example, a targeting peptidomimetic conjugated to a detectable label (e.g., a near-IR fluorescence spectroscopy label such as Cy5.5) allows live in vivo visualization. Such a targeting peptidomimetic can be administered to an animal, and the presence of the targeting peptidomimetic in an organ can be detected, thus allowing determination of the rate and amount of accumulation of the targeting peptidomimetic in the desired organ. In other embodiments, the targeting peptidomimetic can be labelled with a radioactive isotope (e.g., ¹²⁵I) The targeting peptidomimetic is then administered to an animal. After a period of time, the animal is sacrificed and the organs are extracted.

The amount of radioisotope in each organ can then be measured using any means known in the art. By comparing the amount of a labeled candidate targeting peptidomimetic in a particular organ relative to the amount of a labeled control targeting peptidomimetic, the ability of the candidate targeting peptidomimetic to access and accumulate in a particular tissue can be ascertained. Appropriate negative controls include any targeting peptidomimetic known not to be efficiently transported into a particular cell type (e.g., a peptide related to Angiopep that does not cross the BBB or any other peptide).

Additional sequences are described in U.S. Pat. Nos. 5,807,980, 5,780,265, 5,118,668, herein incorporated by reference

### Protected Amino Acids

Targeting peptidomimetics or B having the amino acid sequence of formula I or any one of formula Ia-Ie contain at least one N_{ε}-protected lysine residue. Any N-protecting group can be used to protect the free amino group of the lysine residue.

The term “N-protecting group,” as used herein, refers to a protecting group intended to protect an amino group against undesirable reactions during synthetic procedures, while being stable to those conditions. Suitable N-protecting groups are known in the art. Commonly used N-protecting groups are disclosed in Greene, “Protective Groups in Organic Synthesis,” 3^{rd} Edition (John Wiley & Sons, New York, 1999), which is incorporated herein by reference. N-protecting groups include acyl, aryloyl, or carbamyl groups such as formyl, acetyl, propionyl, pivaloyl, t-butylacetyl, 2-chloroacetyl, 2-bromoacetyl, trifluoroacetyl, trichloroacetyl, phthalyl, o-nitrophenoxyacetyl, α-chlorobutyryl, benzoyl, 4-chlorobenzoyl, 4-bromobenzoyl, 4-nitrobenzoyl; sulfonyl-containing groups such as benzenesulfonyl, p-toluenesulfonyl, and the like; carbamate forming groups such as benzyloxycarbonyl, p-chlorobenzyloxycarbonyl, p-methoxybenzyloxycarbonyl, p-nitrobenzyloxycarbonyl, 2-nitrobenzyloxycarbonyl, p-bromobenzyloxycarbonyl, 3,4-dimethoxybenzyloxycarbonyl, 3,5-dimethoxybenzyl oxycarbonyl, 2,4-dimethoxybenzyloxycarbonyl, 4-methoxybenzyloxycarbonyl, 2-nitro-4,5-dimethoxybenzyloxycarbonyl, 3,4,5-trimethoxybenzyloxycarbonyl, 1-(p-biphenylyl)-1-methylethoxycarbonyl, α,α-dimethyl-3,5-dimethoxybenzyloxycarbonyl, benzhydryloxy carbonyl, t-butyloxycarbonyl, diisopropylmethoxycarbonyl, isopropyloxycarbonyl, ethoxycarbonyl, methoxycarbonyl, allyloxycarbonyl, 2,2,2,-trichloroethoxycarbonyl, phenoxycarbonyl, 4-nitrophenoxy carbonyl, fluorenyl-9-methoxycarbonyl, cyclopentyloxycarbonyl, adamantyloxycarbonyl, cyclohexyloxycarbonyl, phenylthiocarbonyl, and the like, alkaryl groups such as benzyl, triphenylmethyl, benzyloxymethyl, and the like and silyl groups such as trimethylsilyl, and the like. Preferred N-protecting groups are formyl, acetyl, benzoyl, pivaloyl, t-butylacetyl, alanyl, phenylsulfonyl, benzyl, t-butyloxycarbonyl (Boc), and benzyloxycarbonyl (Cbz). In a most particular embodiment, the N-protecting group is acetyl.

### Polypeptide Derivatives and Peptidomimetics

The targeting peptidomimetic, and the enzyme analog may have a modified amino acid sequence. In the case of the targeting peptidomimetic, one modification is the inclusion of an N_{ε}-protected lysine residue. Further modifications to the targeting peptidomimetic are permitted. One or more modifications to the enzyme analog are also permitted.

Modifications include those by natural processes, such as posttranslational processing, or by chemical modification techniques known in the art. Modifications may occur anywhere in a polypeptide including the polypeptide backbone, the amino acid side chains and the amino- or carboxy-terminus. The same type of modification may be present in the same or varying degrees at several sites in a given polypeptide, and a polypeptide may contain more than one type of modification.

In one embodiment, the peptidomimetic has a modified backbone in which one or more peptide linkages are replaced by linkages such as —CH₂NH—, —CH₂S—, —CH₂—CH₂—, —CH═CH— (cis and trans), —CH₂SO—, —CH(OH)CH₂—, —COCH₂— etc., by methods well known in the art (Spatola, Peptide Backbone Modifications, Vega Data, 1:267, 1983; Spatola et al., Life Sci. 38:1243-1249, 1986; Hudson et al., Int. J. Pept. Res. 14:177-185, 1979; and Weinstein, 1983, Chemistry and Biochemistry, of Amino Acids, Peptides and Proteins, Weinstein eds, Marcel Dekker, New York). Such polypeptide mimetics may have significant advantages over naturally occurring polypeptides including more economical production, greater chemical stability, enhanced pharmacological properties (e.g., half-life, absorption, potency, and efficiency), reduced antigenicity, and others.

Polypeptides may be branched as a result of ubiquitination, and they may be cyclic, with or without branching. Cyclic, branched, and branched cyclic polypeptides may result from posttranslational natural processes or may be made synthetically.

A cyclic derivative containing an intramolecular disulfide bond may be prepared by conventional solid phase synthesis while incorporating suitable S-protected cysteine or homocysteine residues at the positions selected for cyclization such as the amino and carboxy termini (Sah et al., J. Pharm. Pharmacol. 48:197, 1996). Following completion of the chain assembly, cyclization can be performed either (1) by selective removal of the S-protecting group with a consequent on-support oxidation of the corresponding two free SH-functions, to form a S—S bonds, followed by conventional removal of the product from the support and appropriate purification procedure or (2) by removal of the polypeptide from the support along with complete side chain de-protection, followed by oxidation of the free SH-functions in highly dilute aqueous solution. Cyclic polypeptides have no free N- or C-termini. Accordingly, they are not susceptible to proteolysis by exopeptidases, although they are, of course, susceptible to endopeptidases, which do not cleave at polypeptide termini. The amino acid sequences of cyclic polypeptides are usually identical to the sequences of the polypeptides to which they correspond, except for their circular structure.

Another effective approach to confer resistance to peptidases acting on the N-terminal or C-terminal residues of a polypeptide is to add chemical groups at the polypeptide termini, such that the modified polypeptide is no longer a substrate for the peptidase. One such chemical modification is glycosylation of the polypeptides at either or both termini. Certain chemical modifications, in particular N-terminal glycosylation, have been shown to increase the stability of polypeptides in human serum (Powell et al., Pharm. Res. 10:1268-1273, 1993). Other Chemical Modifications which Enhance Serum Stability include, but are not limited to, the addition of an N-terminal alkyl group, consisting of a lower alkyl of from one to twenty carbons, such as an acetyl group, and/or the addition of a C-terminal amide or substituted amide group. In particular, the present invention includes modified polypeptides consisting of polypeptides bearing an N-terminal acetyl group and/or a C-terminal amide group.

Other modifications include polypeptide derivatives containing additional chemical moieties not normally part of the polypeptide, provided that the derivative retains the desired functional activity of the polypeptide. Examples include (1) N-acyl derivatives of the amino terminal or of another free amino group, wherein the acyl group may be an alkanoyl group (e.g., acetyl, hexanoyl, octanoyl) an aroyl group (e.g., benzoyl) or a blocking group such as F-moc (fluorenylmethyl-O—CO—); (2) esters of the carboxy terminal or of another free carboxy or hydroxyl group; (3) amide of the carboxy-terminal or of another free carboxyl group produced by reaction with ammonia or with a suitable amine; (4) phosphorylated derivatives; (5) derivatives conjugated to an antibody or other biological ligand. Further examples include pegylation, acetylation, acylation, addition of acetomidomethyl (Acm) group, ADP-ribosylation, alkylation, amidation, biotinylation, carbamoylation, carboxyethylation, esterification, covalent attachment to flavin, covalent attachment to a heme moiety, covalent attachment of a nucleotide or nucleotide derivative, covalent attachment of drug, covalent attachment of a marker (e.g., fluorescent or radioactive), covalent attachment of a lipid or lipid derivative, covalent attachment of phosphatidylinositol, cross-linking, cyclization, disulfide bond formation, demethylation, formation of covalent crosslinks, formation of cystine, formation of pyroglutamate, formylation, gamma-carboxylation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation and ubiquitination.

Polypeptides made synthetically can include substitutions of amino acids not naturally encoded by DNA (e.g., non-naturally occurring or unnatural amino acid). Examples of non-naturally occurring amino acids include D-amino acids, N-protected amino acids, an amino acid having an acetylaminomethyl group attached to a sulfur atom of a cysteine, a pegylated amino acid, the omega amino acids of the formula NH₂(CH₂)ₙCOOH wherein n is 2-6, neutral nonpolar amino acids, such as sarcosine, t-butyl alanine, t-butyl glycine, N-methyl isoleucine, and norleucine. Phenylglycine may substitute for Trp, Tyr, or Phe; citrulline and methionine sulfoxide are neutral nonpolar, cysteic acid is acidic, and ornithine is basic. Proline may be substituted with hydroxyproline and retain the conformation conferring properties.

In one embodiment, the targeting peptidomimetic (B) or enzyme analog may contain D-amino acids. Systematic substitution of one or more amino acids of a consensus sequence with D-amino acid of the same type (e.g., an enantiomer; D-lysine in place of L-lysine) may be used to generate more stable polypeptides. Thus, an analog or peptidomimetic as described herein may be all L-, all D-, or mixed D, L polypeptides. The presence of an N-terminal or C-terminal D-amino acid increases the in vivo stability of a polypeptide because peptidases cannot utilize a D-amino acid as a substrate (Powell et al., Pharm. Res. 10:1268-1273, 1993). Reverse-D polypeptides are polypeptides containing D-amino acids, arranged in a reverse sequence relative to a polypeptide containing L-amino acids. Thus, the C-terminal residue of an L-amino acid polypeptide becomes N-terminal for the D-amino acid polypeptide, and so forth. Reverse D-polypeptides retain the same tertiary conformation and therefore the same activity, as the L-amino acid polypeptides, but are more stable to enzymatic degradation in vitro and in vivo, and thus have greater therapeutic efficacy than the original polypeptide (Brady and Dodson, Nature 368:692-693, 1994 and Jameson et al., Nature 368:744-746, 1994).

In certain embodiments, the modification(s) do not destroy significantly a desired biological activity (e.g., ability to cross the BBB or enzymatic activity). The modification may reduce (e.g., by at least 5%, 10%, 20%, 25%, 35%, 50%, 60%, 70%, 75%, 80%, 90%, or 95%), may have no effect, or may increase (e.g., by at least 5%, 10%, 25%, 50%, 100%, 200%, 500%, or 1000%) the biological activity of the original polypeptide. The modified polypeptides may have or may optimize a characteristic of a polypeptide, such as in vivo stability, bioavailability, toxicity, immunological activity, immunological identity, and conjugation properties. In one embodiment, the modified polypeptides may have increased stability to proteases. Serum proteases have specific substrate requirements, including L-amino acids and peptide bonds for cleavage. Furthermore, exopeptidases, which represent the most prominent component of the protease activity in serum, usually act on the first peptide bond of the polypeptide and require a free N-terminus (Powell et al., Pharm. Res. 10:1268-1273, 1993). As discussed above, certain modified polypeptides may retain the structural characteristics of the original L-amino acid polypeptides, but advantageously are not readily susceptible to cleavage by protease and/or exopeptidases.

Substantial modifications in function or immunological identity are accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, or (c) the bulk of the side chain.

Other derivatives included in the present invention are dual polypeptides consisting of two of the same, or two different polypeptides, as described herein, covalently linked to one another either directly or through a spacer, such as by a short stretch of alanine residues or by a putative site for proteolysis (e.g., by cathepsin, see e.g., U.S. Pat. No. 5,126,249 and European Patent No. 495 049). Multimers of the polypeptides described herein consist of a polymer of molecules formed from the same or different polypeptides or derivatives thereof.

The present invention also encompasses polypeptide derivatives that are chimeric or fusion proteins containing a polypeptide described herein, or fragment thereof, linked at its amino- or carboxy-terminal end, or both, to an amino acid sequence of a different protein. Such a chimeric or fusion protein may be produced by recombinant expression of a nucleic acid encoding the protein. For example, a chimeric or fusion protein may contain at least 6 amino acids shared with one of the described polypeptides which desirably results in a chimeric or fusion protein that has an equivalent or greater functional activity.

### Assays to Identify Peptidomimetics

As described above, non-peptidyl compounds generated to replicate the backbone geometry and pharmacophore display (peptidomimetics) of the polypeptides described herein often possess attributes of greater metabolic stability, higher potency, longer duration of action, and better bioavailability.

Peptidomimetics compounds can be obtained using any of the numerous approaches in combinatorial library methods known in the art, including biological libraries, spatially addressable parallel solid phase or solution phase libraries, synthetic library methods requiring deconvolution, the ‘one-bead one-compound’ library method, and synthetic library methods using affinity chromatography selection. The biological library approach is limited to peptide libraries, while the other four approaches are applicable to peptide, non-peptide oligomer, or small molecule libraries of compounds (Lam, Anticancer Drug Des. 12:145, 1997). Examples of methods for the synthesis of molecular libraries can be found in the art, for example, in: DeWitt et al. (Proc. Natl. Acad. Sci. USA 90:6909, 1993); Erb et al. (Proc. Natl. Acad. Sci. USA 91:11422, 1994); Zuckermann et al. (J. Med. Chem. 37:2678, 1994); Cho et al. (Science 261:1303, 1993); Carell et al. (Angew. Chem, Int. Ed. Engl. 33:2059, 1994 and ibid 2061); and in Gallop et al. (Med. Chem. 37:1233, 1994). Libraries of compounds may be presented in solution (e.g., Houghten, Biotechniques 13:412-421, 1992) or on beads (Lam, Nature 354:82-84, 1991), chips (Fodor, Nature 364:555-556, 1993), bacteria or spores (U.S. Pat. No. 5,223,409), plasmids (Cull et al., Proc. Natl. Acad. Sci. USA 89:1865-1869, 1992) or on phage (Scott and Smith, Science 249:386-390, 1990), or luciferase, and the enzymatic label detected by determination of conversion of an appropriate substrate to product.

Once a polypeptide as described herein is identified, it can be isolated and purified by any number of standard methods including, but not limited to, differential solubility (e.g., precipitation), centrifugation, chromatography (e.g., affinity, ion exchange, and size exclusion), or by any other standard techniques used for the purification of peptides, peptidomimetics, or proteins. The functional properties of an identified polypeptide of interest may be evaluated using any functional assay known in the art. Desirably, assays for evaluating downstream receptor function in intracellular signaling are used (e.g., cell proliferation).

For example, the peptidomimetics compounds of the present invention may be obtained using the following three-phase process: (1) scanning the polypeptides described herein to identify regions of secondary structure necessary for targeting the particular cell types described herein; (2) using conformationally constrained dipeptide surrogates to refine the backbone geometry and provide organic platforms corresponding to these surrogates; and (3) using the best organic platforms to display organic pharmocophores in libraries of candidates designed to mimic the desired activity of the native polypeptide. In more detail the three phases are as follows. In phase 1, the lead candidate polypeptides are scanned and their structure abridged to identify the requirements for their activity. A series of polypeptide analogs of the original are synthesized. In phase 2, the best polypeptide analogs are investigated using the conformationally constrained dipeptide surrogates. Indolizidin-2-one, indolizidin-9-one and quinolizidinone amino acids (I²aa, I⁹aa and Qaa respectively) are used as platforms for studying backbone geometry of the best peptide candidates. These and related platforms (reviewed in Halab et al., Biopolymers 55:101-122, 2000 and Hanessian et al., Tetrahedron 53:12789-12854, 1997) may be introduced at specific regions of the polypeptide to orient the pharmacophores in different directions. Biological evaluation of these analogs identifies improved lead polypeptides that mimic the geometric requirements for activity. In phase 3, the platforms from the most active lead polypeptides are used to display organic surrogates of the pharmacophores responsible for activity of the native peptide. The pharmacophores and scaffolds are combined in a parallel synthesis format. Derivation of polypeptides and the above phases can be accomplished by other means using methods known in the art.

Structure function relationships determined from the polypeptides, polypeptide derivatives, peptidomimetics or other small molecules described herein may be used to refine and prepare analogous molecular structures having similar or better properties. Accordingly, the compounds of the present invention also include molecules that share the structure, polarity, charge characteristics and side chain properties of the polypeptides described herein.

In summary, based on the disclosure herein, those skilled in the art can develop peptides and peptidomimetics screening assays which are useful for identifying compounds for targeting an agent to particular cell types (e.g., those described herein). The assays of this invention may be developed for low-throughput, high-throughput, or ultra-high throughput screening formats. Assays of the present invention include assays amenable to automation.

### Treatment of Lysosomal Storage Disorders

The present invention also features methods for treatment of lysosomal storage disorders such as MPS-II. MPS-II is characterized by cellular accumulation of glycosaminoglycans (GAG) which results from the inability of the individual to break down these products. MPSII may be treated with compounds of formula II or III, or the populations of formula IIa or IIIa,

In certain embodiments, treatment is performed on a subject who has been diagnosed with a mutation in the IDS gene, but does not yet have disease symptoms (e.g., an infant or subject under the age of 2). In other embodiments, treatment is performed on an individual who has at least one MPS-II symptom (e.g., any of those described herein).

MPS-II is generally classified into two general groups, severe disease and attenuated disease. The present invention can involve treatment of subjects with either type of disease. Severe disease is characterized by CNS involvement. In severe disease the cognitive decline, coupled with airway and cardiac disease, usually results in death before adulthood. The attenuated form of the disease general involves only minimal or no CNS involvement. In both severe and attenuated disease, the non-CNS symptoms can be as severe as those with the “severe” form.

Initial MPS-II symptoms begin to manifest themselves from about 18 months to about four years of age and include abdominal hernias, ear infections, runny noses, and colds. Symptoms include coarseness of facial features (e.g., prominent forehead, nose with a flattened bridge, and an enlarged tongue), large head (macrocephaly), enlarged abdomen, including enlarged liver (heptaomegaly) and enlarged spleen (slenomegaly), and hearing loss. The methods of the invention may involve treatment of subjects having any of the symptoms described herein. MPS-II also results in joint abnormalities, related to thickening of bones.

Treatment may be performed in a subject of any age, starting from infancy to adulthood. Subjects may begin treatment at birth, six months, or 1, 2, 3, 4, 5, 6, 7, 8, 9, 10, 11, 12, 13, 15, or 18 years of age.

### Administration and Dosage

The present invention also features pharmaceutical compositions that contain a therapeutically effective amount of a compound of the invention. The composition can be formulated for use in a variety of drug delivery systems. One or more physiologically acceptable excipients or carriers can also be included in the composition for proper formulation. Suitable formulations for use in the present invention are found in Remington's Pharmaceutical Sciences, Mack Publishing Company, Philadelphia, Pa., 17th ed., 1985. For a brief review of methods for drug delivery, see, e.g., Langer (Science 249:1527-1533, 1990).

The pharmaceutical compositions are intended for parenteral, intranasal, topical, oral, or local administration, such as by a transdermal means, for prophylactic and/or therapeutic treatment. The pharmaceutical compositions can be administered parenterally (e.g., by intravenous, intramuscular, or subcutaneous injection), or by oral ingestion, or by topical application or intraarticular injection at areas affected by the vascular or cancer condition. Additional routes of administration include intravascular, intra-arterial, intratumor, intraperitoneal, intraventricular, intraepidural, as well as nasal, ophthalmic, intrascleral, intraorbital, rectal, topical, or aerosol inhalation administration. Sustained release administration is also specifically included in the invention, by such means as depot injections or erodible implants or components. Thus, the invention provides compositions for parenteral administration that include the above mention agents dissolved or suspended in an acceptable carrier, preferably an aqueous carrier, e.g., water, buffered water, saline, PBS, and the like. The compositions may contain pharmaceutically acceptable auxiliary substances as required to approximate physiological conditions, such as pH adjusting and buffering agents, tonicity adjusting agents, wetting agents, detergents and the like. The invention also provides compositions for oral delivery, which may contain inert ingredients such as binders or fillers for the formulation of a tablet, a capsule, and the like. Furthermore, this invention provides compositions for local administration, which may contain inert ingredients such as solvents or emulsifiers for the formulation of a cream, an ointment, and the like.

These compositions may be sterilized by conventional sterilization techniques, or may be sterile filtered. The resulting aqueous solutions may be packaged for use as is, or lyophilized, the lyophilized preparation being combined with a sterile aqueous carrier prior to administration. The pH of the preparations typically will be between 3 and 11, more preferably between 5 and 9 or between 6 and 8, and most preferably between 7 and 8, such as 7 to 7.5. The resulting compositions in solid form may be packaged in multiple single dose units, each containing a fixed amount of the above-mentioned agent or agents, such as in a sealed package of tablets or capsules. The composition in solid form can also be packaged in a container for a flexible quantity, such as in a squeezable tube designed for a topically applicable cream or ointment.

The compositions containing an effective amount can be administered for prophylactic or therapeutic treatments. In prophylactic applications, compositions can be administered to a subject diagnosed as having mutation associated with a lysosomal storage disorder (e.g., a mutation in the IDS gene). Compositions of the invention can be administered to the subject (e.g., a human) in an amount sufficient to delay, reduce, or preferably prevent the onset of the disorder. In therapeutic applications, compositions are administered to a subject (e.g., a human) already suffering from a lysosomal storage disorder (e.g., MPS-II) in an amount sufficient to cure or at least partially arrest the symptoms of the disorder and its complications. An amount adequate to accomplish this purpose is defined as a “therapeutically effective amount,” an amount of a compound sufficient to substantially improve at least one symptom associated with the disease or a medical condition. For example, in the treatment of a lysosomal storage disease, an agent or compound that decreases, prevents, delays, suppresses, or arrests any symptom of the disease or condition would be therapeutically effective. A therapeutically effective amount of an agent or compound is not required to cure a disease or condition but will provide a treatment for a disease or condition such that the onset of the disease or condition is delayed, hindered, or prevented, or the disease or condition symptoms are ameliorated, or the term of the disease or condition is changed or, for example, is less severe or recovery is accelerated in an individual.

Amounts effective for this use may depend on the severity of the disease or condition and the weight and general state of the subject. Idursulfase is recommended for weekly intravenous administration of 0.5 mg/kg. The compound of formula II or III, or the population of formula IIa or IIIa, may, for example, be administered at an equivalent dosage (i.e., accounting for the additional molecular weight of the fusion protein vs. idursulfase) and frequency. The compounds of formula II or III, or the populations of formula IIa or IIIa, may be administered at an iduronase equivalent dose, e.g., 0.01, 0.05, 0.1, 0.5, 0.1, 0.2, 0.3, 0.4, 0.5, 0.75, 1.0, 1.25, 1.5, 2.0, 2.5, 3.0, 4.0, or 5 mg/kg weekly, twice weekly, every other day, daily, or twice daily. The therapeutically effective amount of the compositions of the invention and used in the methods of this invention applied to mammals (e.g., humans) can be determined by the ordinarily-skilled artisan with consideration of individual differences in age, weight, and the condition of the mammal. Because certain compounds of the invention exhibit an enhanced ability to cross the BBB and to enter lysosomes, the dosage of the compounds of the invention can be lower than (e.g., less than or equal to about 90%, 75%, 50%, 40%, 30%, 20%, 15%, 12%, 10%, 8%, 7%, 6%, 5%, 4%, 3%, 2%, 1%, 0.5%, or 0.1% of) the equivalent dose of required for a therapeutic effect of the unconjugated agent. The agents of the invention are administered to a subject (e.g., a mammal, such as a human) in an effective amount, which is an amount that produces a desirable result in a treated subject (e.g., reduction of GAG accumulation). Therapeutically effective amounts can also be determined empirically by those of skill in the art.

Single or multiple administrations of the compositions of the invention including an effective amount can be carried out with dose levels and pattern being selected by the treating physician. The dose and administration schedule can be determined and adjusted based on the severity of the disease or condition in the subject, which may be monitored throughout the course of treatment according to the methods commonly practiced by clinicians or those described herein.

The compounds of the present invention may be used in combination with either conventional methods of treatment or therapy or may be used separately from conventional methods of treatment or therapy.

When the compounds of this invention are administered in combination therapies with other agents, they may be administered sequentially or concurrently to an individual. Alternatively, pharmaceutical compositions according to the present invention may be comprised of a combination of a compound of the present invention in association with a pharmaceutically acceptable excipient, as described herein, and another therapeutic or prophylactic agent known in the art.

The following examples are intended to illustrate, rather than limit, the invention.

### Example 1

### Synthesis of AN₂[(Ac)Lys^{10,15}]-PEG-NHS ester 1

AN₂[(Ac)Lys^{10,15}] 3 was synthesized using standard solid phase peptide synthesis techniques with (Ac)Lys from Chem-Impex International, II, USA.

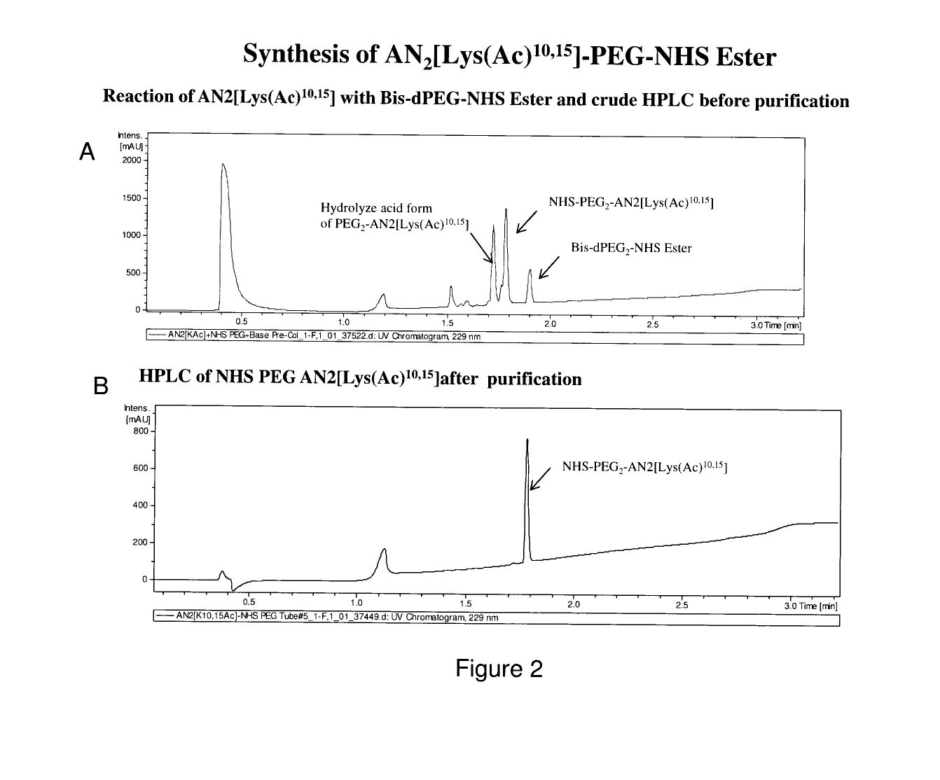

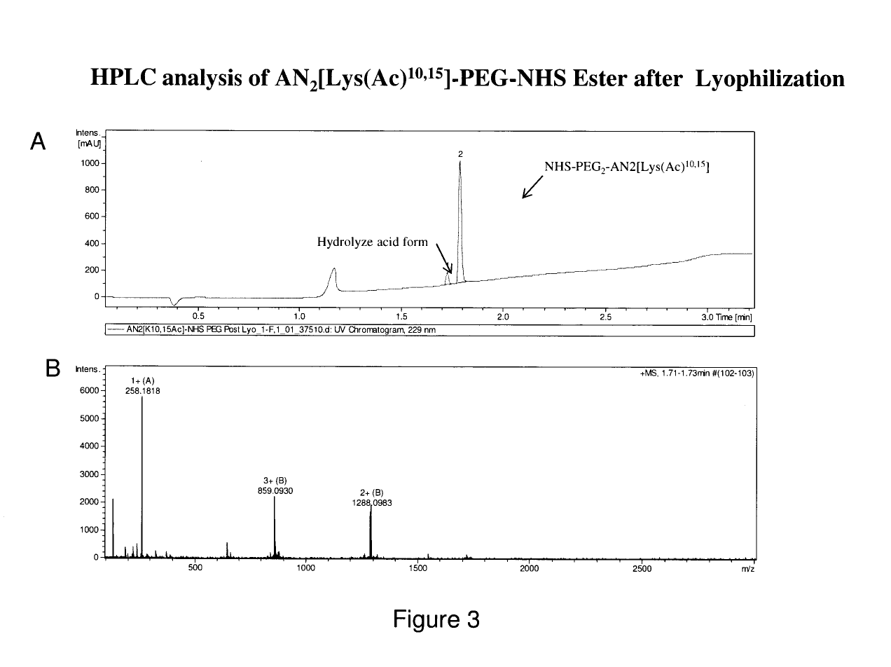

Bis-N-succinimidyl-(diethylene glycol) ester 2 was reacted with AN₂[(Ac)Lys^{10,15}] 3 to provide AN₂[(Ac)Lys^{10,15}]-PEG-NHS ester 1 as shown in scheme 1. FIG. 2A shows the HPLC trace of the crude reaction mixture and FIG. 2B shows the HPLC trace after purification. The purified AN₂[(Ac)Lys^{10,15}]-PEG-NHS ester 1 was lyophilized resulting in a 24.2% yield with 92.3% purity post-lyophilization (FIGS. 3A and 3B).

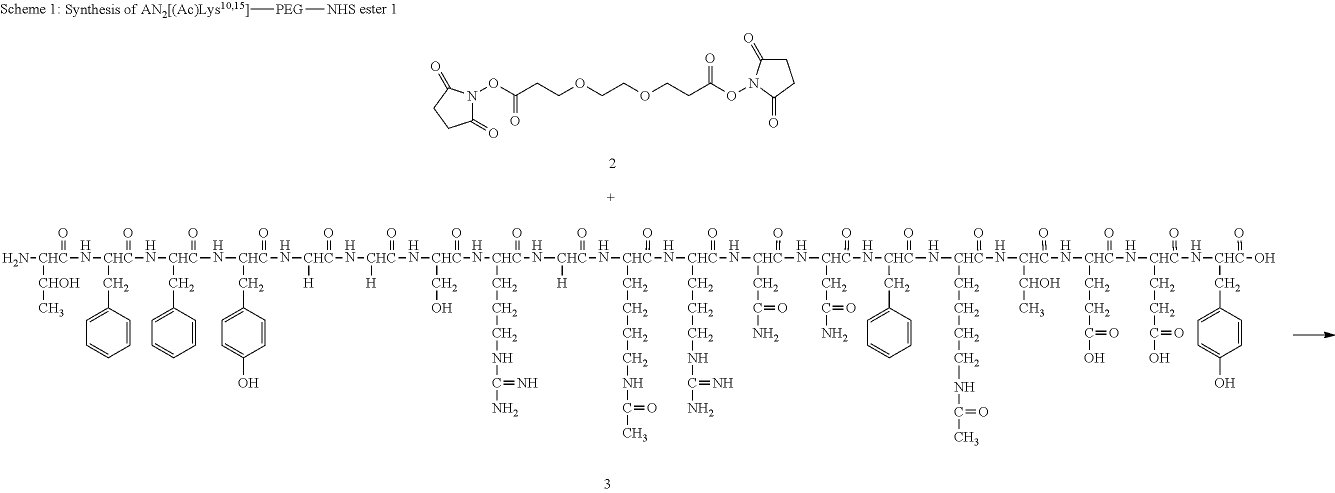

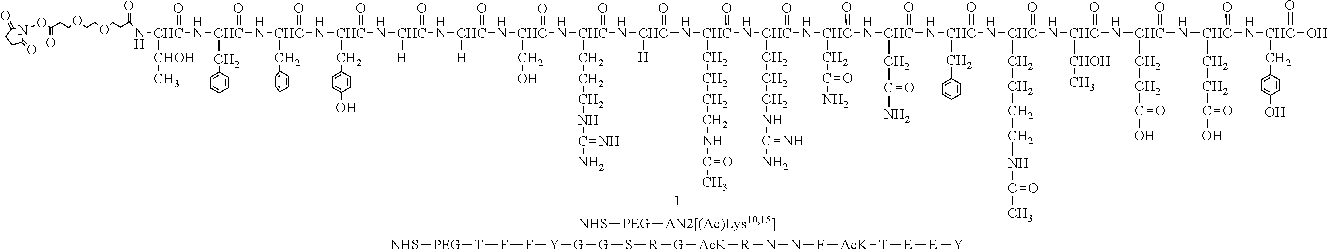

### Example 2

### Synthesis of AN₂-4D[(Ac)Lys^{10,15}]-PEG-NHS esters

AN₂-4[(N_{ε}—Ac)Lys^{10,15}]-PEG-NHS esters were synthesized using the methods described in Example 1 with the substitution of Arg⁸, (N_{ε}—Ac)Lys¹⁰, Arg¹¹, and (N_{ε}—Ac)Lys¹⁵ for the corresponding D-amino acids. AN₂-4D[(N_{ε}—Ac)Lys^{10,15}]-PEG₄-NHS ester, AN₂-4D[(N_{ε}—Ac)Lys^{10,15}]-PEG₇-NHS ester, AN₂-4 D[(N_{ε}—Ac)Lys^{10,15}]-PEG₉-NHS ester, and AN₂-4D[(N_{ε}—Ac)Lys^{10,15}]-PEG₁₃-NHS ester were each synthesized by replacing Bis-N-succinimidyl-(diethylene glycol) ester 2 of Example 1 with Bis-N-succinimidyl-(tetraethylene glycol) ester, Bis-N-succinimidyl-(heptaethylene glycol) ester, Bis-N-succinimidyl-(nonaethylene glycol) ester, and Bis-N-succinimidyl-(tridecaethylene glycol) ester respectively.

### Example 3

### Synthesis of 77-18-1, 77-18-2, and 77-18-3

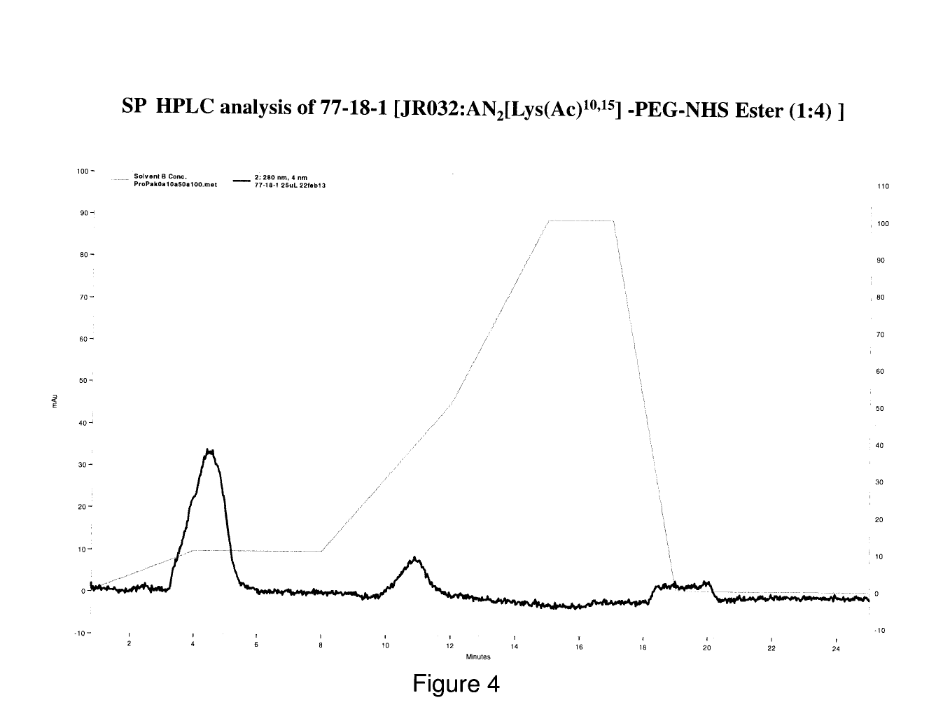

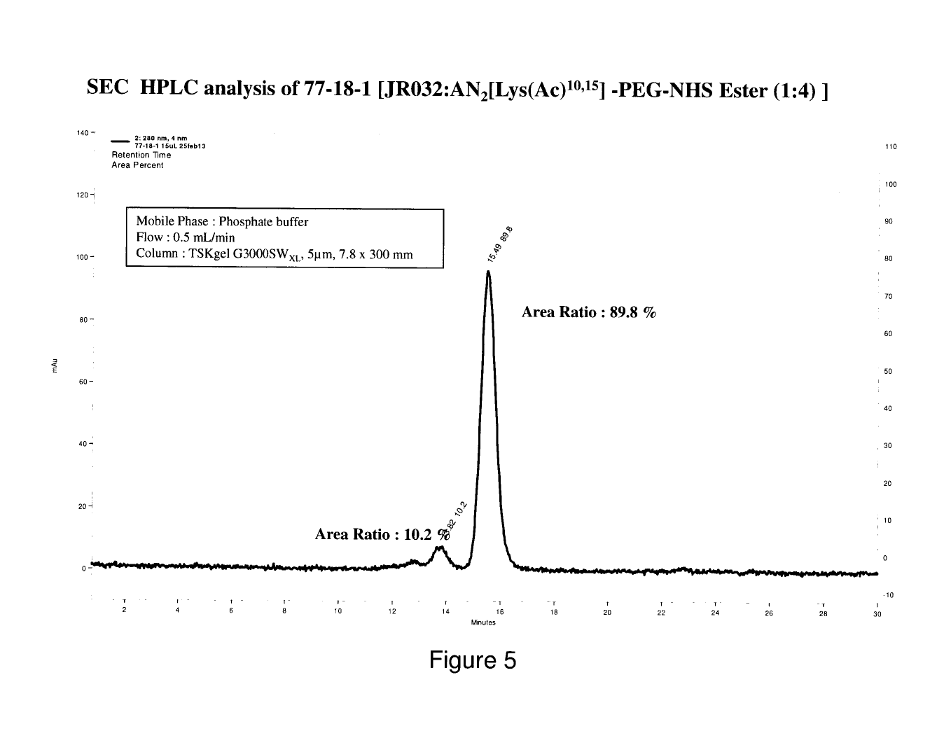

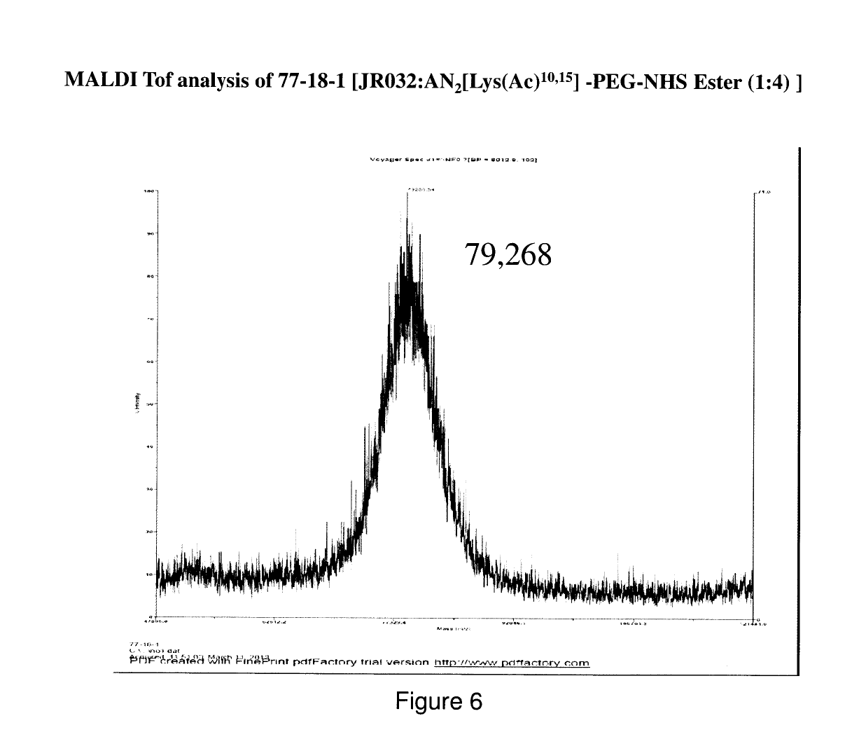

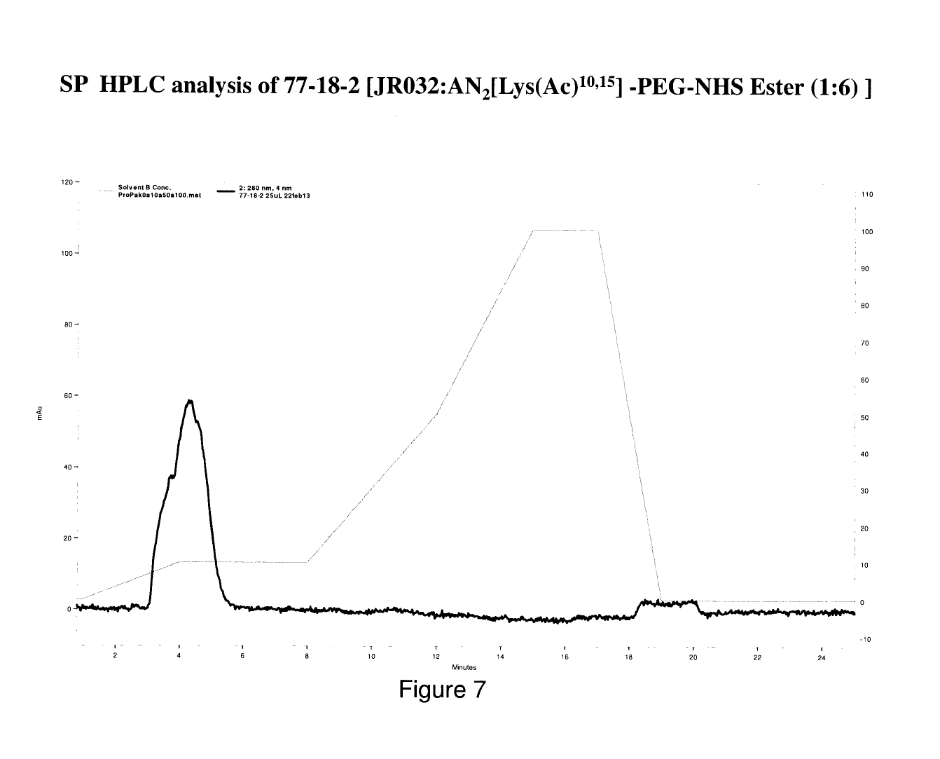

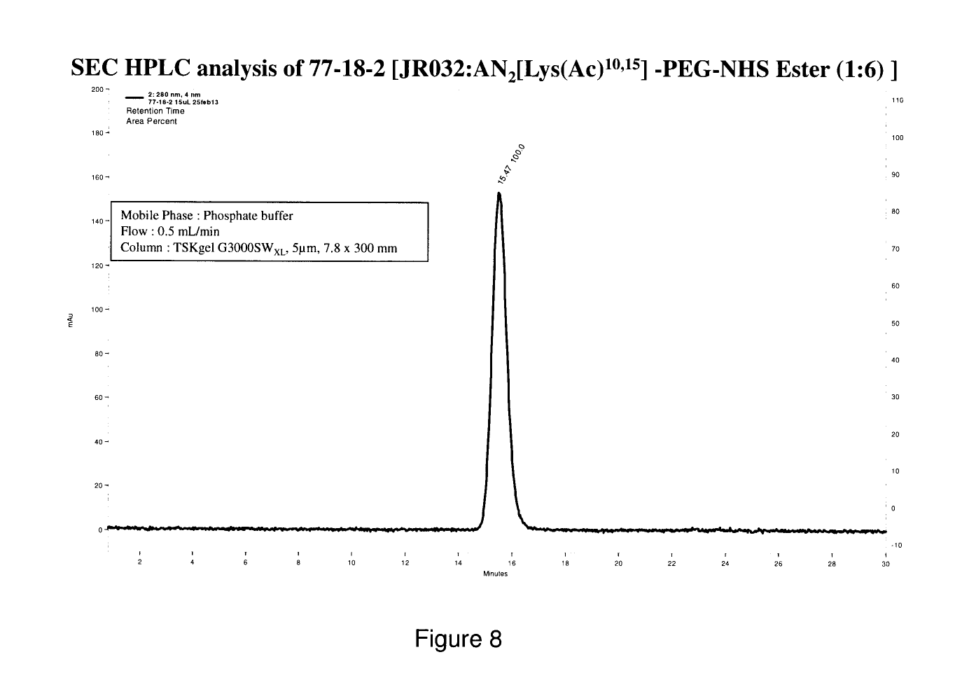

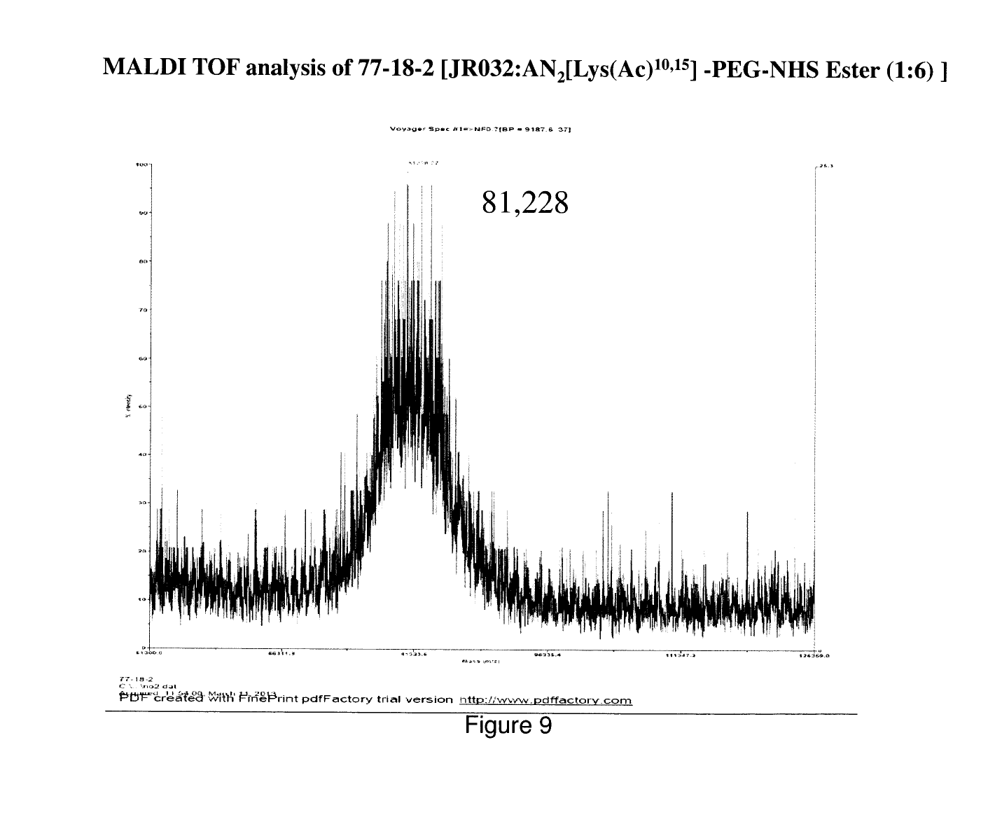

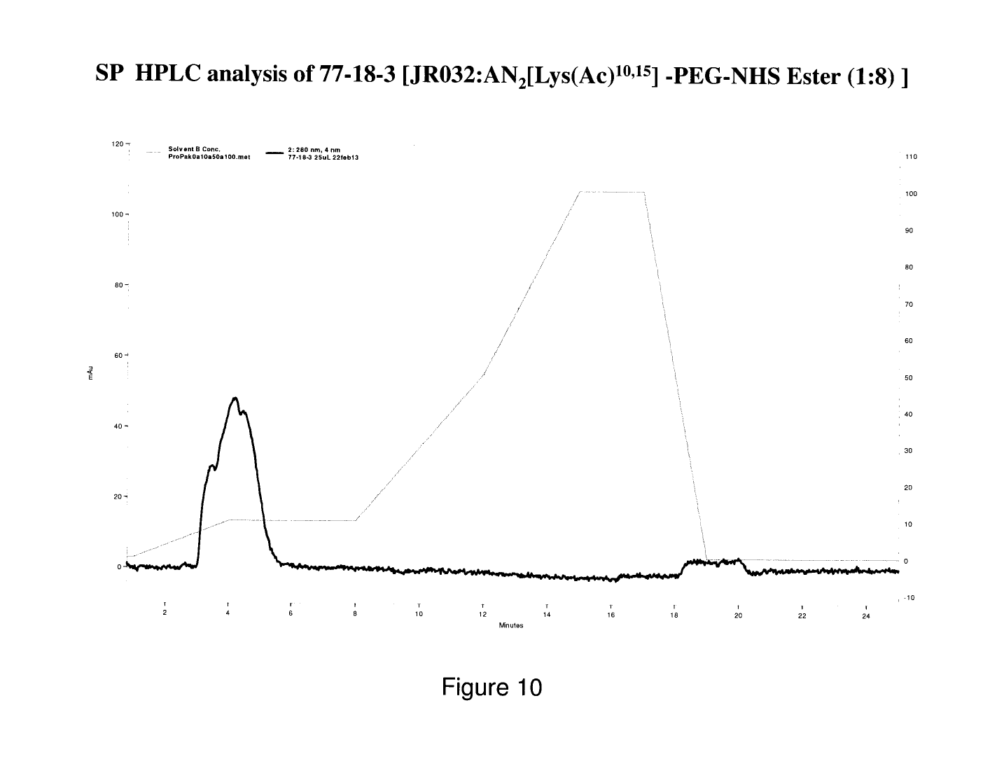

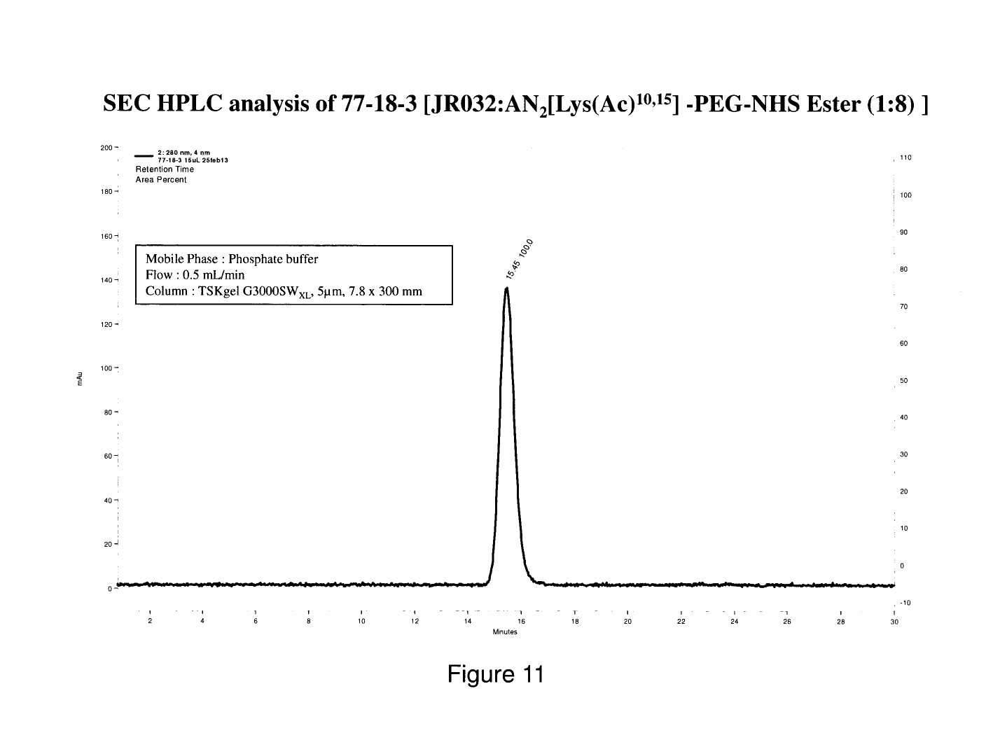

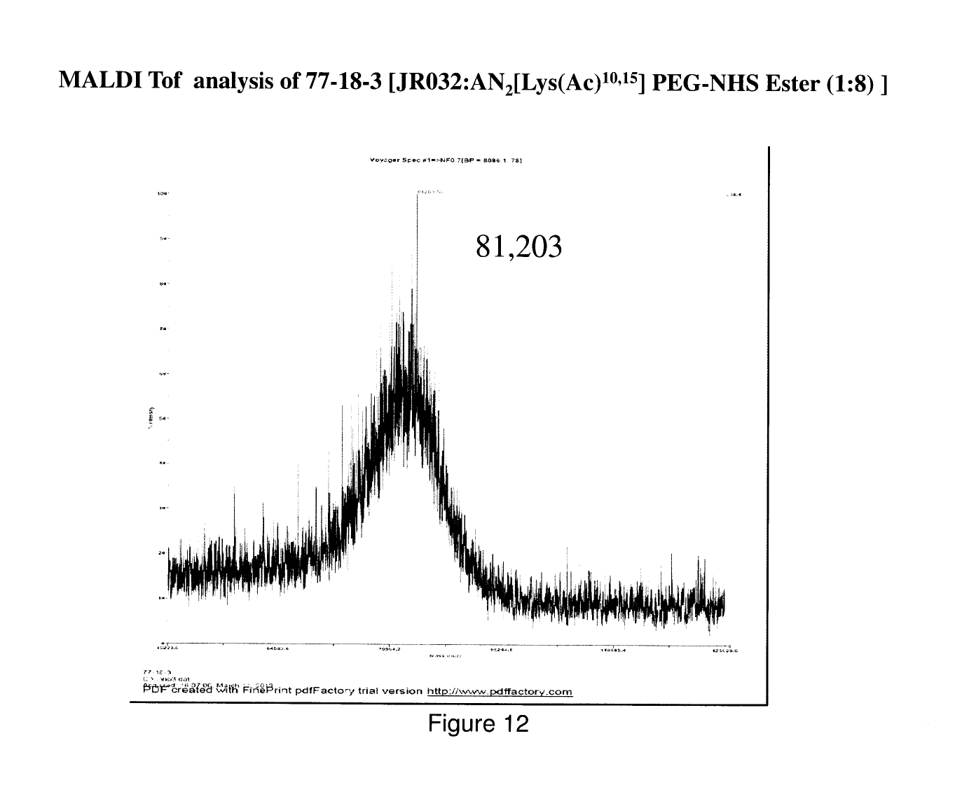

AN2-IDS conjugates 77-18-1, 77-18-2, and 77-18-3 were synthesized by reacting JR-032 with 4, 6, and 8 equivalents of AN₂[(N_{ε}—Ac)Lys^{10,15}]-PEG-NHS ester 1 respectively as shown in Scheme 2. The NH group attached to IDS represents a primary amine group of IDS which has been modified by attachment of an AN₂[(N_{ε}—Ac)Lys^{10,155}]-PEG group. The value of n is the average number of AN₂[(N_{ε}—Ac)Lys^{10,15}]-PEG groups attached to each IDS for the population of compounds in each synthesis. The SP HPLC, SEC HPLC, and MALDI TOF for 77-18-1 are shown in FIGS. 4-6. The SP HPLC (ion exchange chromatography using sulfopropyl SP-HPLC column), SEC HPLC, and MALDI TOF for 77-18-2 are shown in FIGS. 7-9. The SP HPLC, SEC HPLC, and MALDI TOF for 77-18-3 are shown in FIGS. 10-12. Table 3 summarizes the results of these experiments.

TABLE 3
MALDI TOF analysis of AN2-IDS conjugates

| | Ratio | Control | | |
|---|---|---|---|---|
| | JR032:AN₂[(N_{ε}- | IDS | MALDI | Number |
| An2-IDS | Ac)Lys^{10,15}]- | MALDI- | Tof for | of |
| conjugate | PEG-NHS Ester | Tof | conjugates | An2 |
| 77-18-1 | 1:4 | 75,529 | 79,268 | 1.5 |
| 77-18-2 | 1:6 | 75,416 | 81,228 | 2.3 |
| 77-18-3 | 1:8 | 75,000 | 81,203 | 2.3 |

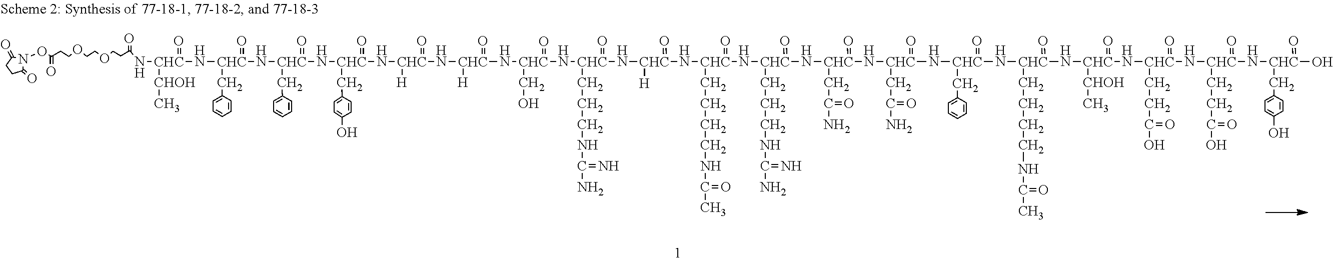

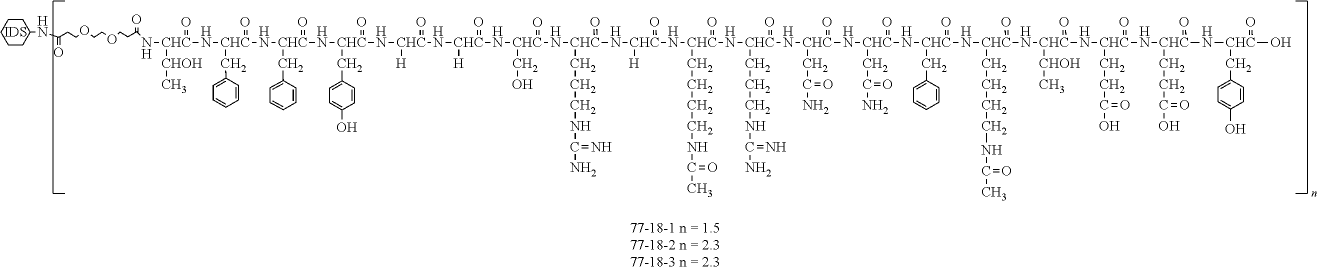

### General Procedure for the Synthesis of AN2-IDS Conjugates:

To JR032 (14.5 mg, 4 mL, 190 nmoles) at a pH of 7.6˜8.00, 8 equivalent of AN₂[(Ac)Lys^{10,15}]-PEG-NHS ester in DMSO (5.07 mg, taking in to consideration 80% peptide content) (146 μl) was added. The solution was manually shaken 3 to 4 times and left at room temperature overnight. The conjugate was purified by Q Sepharose 20 mL column using 20 mM TRIS buffer at pH 7 as binding buffer and 20 mM TRIS and 1M NaCl at pH 7.0 was used as eluent buffer. After purification 25 mL conjugate was collected, concentrated to 5 mL and exchanged with IDS buffer (1×: 137 mM NaCl, 17 mM NaH₂PO₄, 3 mM Na₂HPO₄, at pH-6) by washing 4 times 15 mL with Amicon ultra centrifugal filter (10 kDa cut-off, 3200 rpm) and concentrated to 4 mL to obtain 77-18-3 (14 mg, yield 96.5%).

### Example 4

### Synthesis of ANG3406

ANG3406 was synthesized utilizing the method described above in Example 3 replacing AN₂-[(N_{ε}—Ac)Lys^{10,15}]-PEG-NHS ester 1 with NHS-dPEG₄-AN2-4D[Lys(N_{ε}—Ac)^{10,15}] ester. JR032 was reacted with 8 equivalents of NHS-dPEG₄-AN2-4D[Lys(N_{ε}—Ac)^{10,15}] ester resulting in an average of 1.6 dPEG₄-AN2-4D[Lys(N_{ε}—Ac)^{10,15}] groups conjugated to each enzyme.

The structure of ANG3406 is:

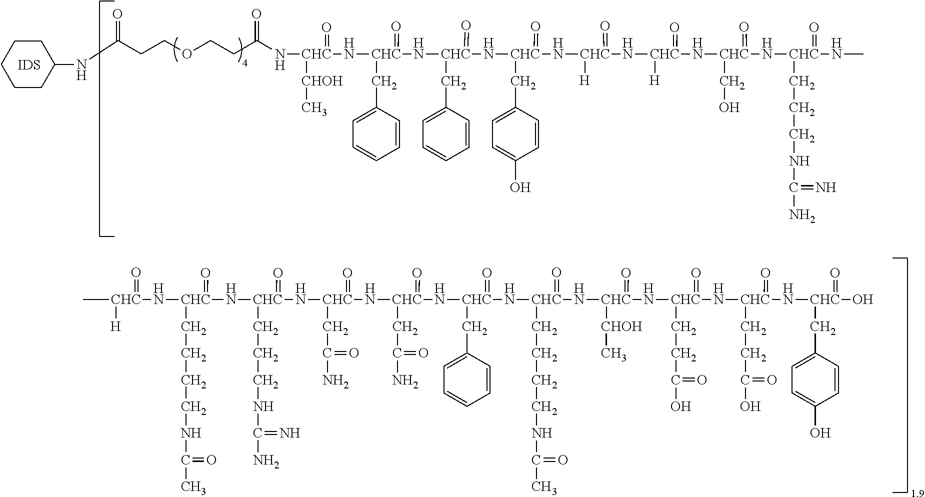

wherein Arg⁸, Lys¹⁰, Arg¹¹, and Lys¹⁵ are substituted with the corresponding D-amino acids.

### Example 5

### In Vitro Enzymatic Activity

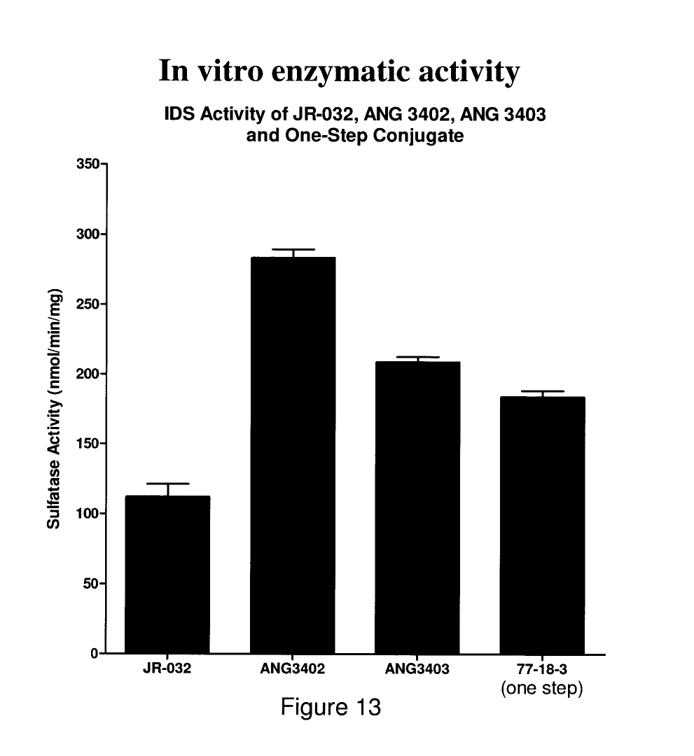

The IDS activity of JR-032, ANG3402, ANG3403, and 77-18-3 were compared (FIG. 13). As shown in FIG. 13, 77-18-3 displayed greater sulfatase activity than the enzyme alone (JR-032).

The structures of ANG3402 and ANG3403 are:

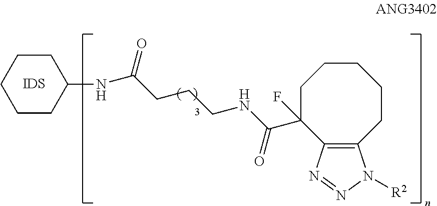

wherein R2 is:

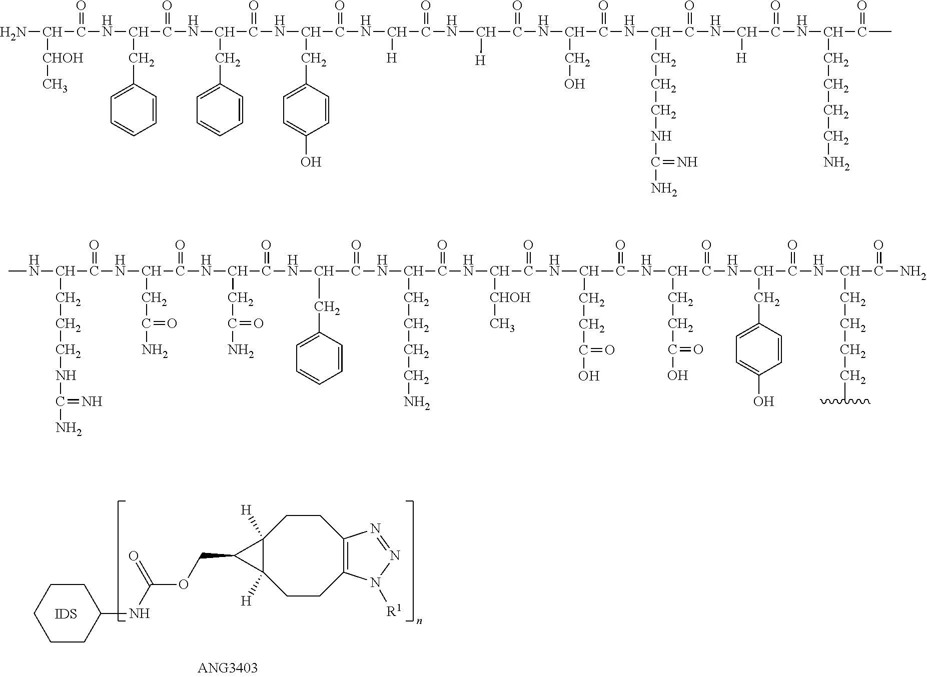

wherein R¹ is:

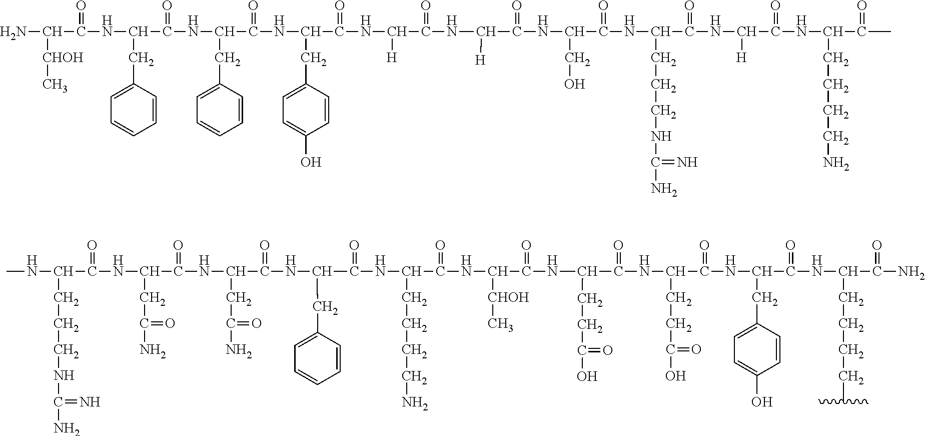

### Protocol for IDS Enzymatic Specific Activity

First, the concentration of proteins in JR-032 and fusion protein samples are determined by microBCA (bicinchoninic acid) (Smith, P. K. et al., 1986, Anal. Biochem., 150(1): 76-85). Test solutions are prepared by diluting JR-032 and fusion proteins 1/200 in Triton-X100 containing diluted buffer. A standard solution is prepared by diluting 1 mL 4-MU (4-methylumbelliferone) Stock Solution (0.01 mol/L) in 11.5 mL of Triton-X100 containing buffer (final concentration 800 μmol/L), followed by preparation of serial dilutions of this standard solution by diluting 500 μL of 800 μmol/L in 500 μL of Triton×100 containing buffer to make a 400 μmol/L solution. The process is repeated to have the following dilutions: 800, 400, 200, 100, 50, 25, 12.5 and 6.25 μmol/L. The solutions are distributed as follows: 10 μL each of the blank solution (Triton-X100 containing diluted buffer) in 2 wells (n=2), standard solution (6.25 μmol/L to 800 μmol/L) in 2 wells (n=2) and the test solutions in 4 wells each (n=4) of a microplate, respectively. Subsequently to each well is added 100 μL of the substrate solution (4-methylumbelliferyl sulfate potassium salt) and the solutions are mixed gently. The plate is covered and placed in an incubator adjusted to 37° C.

After 60 minutes, 190 μL of the stop solution is added to each well and mixed to stop the reaction. The plate is set in the fluorescence plate reader and the fluorescence intensity at excitation wavelength of 355 nm and detection wavelength of 460 nm is determined. The same measurement is performed with the reference material if comparison is required among tests.

### Method of Calculation:

Concentration of 4-MU Produced from the Test Solution:
The concentration of 4-MU, Cu (μmol/L), produced from the test solution was determined using the following formula.

$$Cs = \frac{w}{176.17} \times \frac{{10}^{6}}{50 \times 100}$$

w: Amount (mg) of 4-MU (176.17: Molecular weight of 4-MU)
Cs: Concentration (μmol/L) in the standard solution

$$Cu = Cs ( \frac{Au}{As} )$$

Au: Fluorescence intensity of the test solution
As: Fluorescence intensity of the standard solution
Specific activity of the sample solution:
The specific activity, B (mU/mg), of the sample solution was determined using the following formula.

$$B = \frac{\frac{Cu}{60} \times C \times \frac{50}{0.1}}{P}$$

C: Dilution factor of the desalted test substance
B: Specific activity (mU/mg)
P: Concentration (mg/mL) of proteins in the desalted test substance

### Results

Lysine conjugates were subjected to in vitro enzyme assays with JR-032 as a control. All conjugates retain enzyme activity (see FIG. 13). In some cases, measured activity exceeds that of native IDS. This may result from interference in the protein quantification assay, leading to a lower calculated protein concentration and higher activity/protein or could indicate that modification of the enzyme positively modulates its activity. FIG. 13 also shows results of the in vitro enzymatic activity of a large scale syntheses of 77-18-3 (ANG305 1A).

### Example 6

### Brain Perfusion

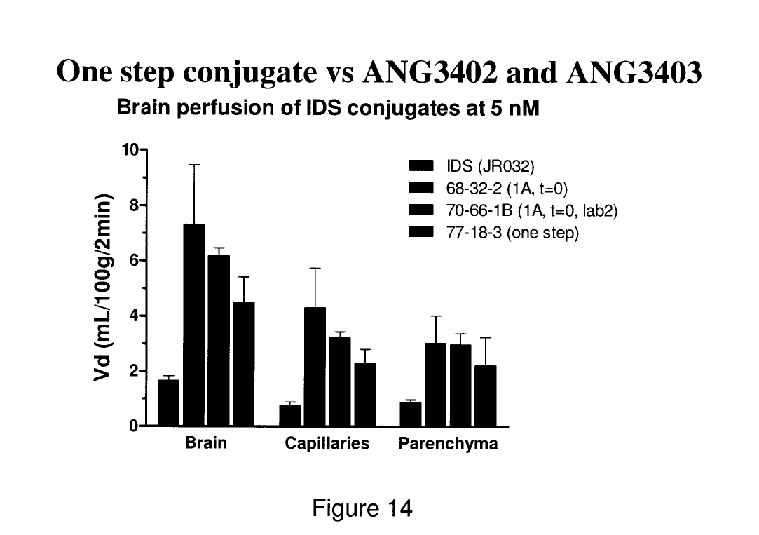

The brain perfusion of JR-032, 70-66-1B (ANG3402), 68-32-2 (ANG3403), and IDS conjugate 77-18-3 was analyzed (FIG. 14). As shown in the graph of FIG. 14, 77-18-3 showed greater volume of distribution in the brain, capillaries, and parenchyma when compared to the enzyme alone (JR-032).

### Protocol for In Situ Brain Perfusion

The in situ mice brain perfusion method was established in the laboratory from the protocol described by Dagenais et al., 2000. Briefly, the surgery was performed on sedated mice, injected intraperitoneal (i.p.) with Ketamine/Xylazine (140/8 mg/kg). The right common carotid artery was exposed and ligated at the level of the bifurcation. The common carotid was then catheterized rostrally with polyethylene tubing (0.30 mm i.d.×0.70 mm o.d.) filled with saline/heparan (25 U/ml) solution mounted on a 26-gauge needle. The studied molecule was radiolabeled with ¹²⁵I in the days preceding the experiment using iodo-Beads from Pierce. Free iodine was removed on gel filtration column followed by extensive dialysis (cut-off 10 kDa). Radiolabeled proteins were dosed using the Bradford assay and JR-032 as the standard.

Prior to surgery, perfusion buffer consisting of KREBS-bicarbonate buffer—9 mM glucose was prepared and incubated at 37° C., pH at 7.4 stabilized with 95% O₂:5% CO₂. A syringe containing radiolabeled compound added to the perfusion buffer was placed on an infusion pump (Harvard pump PHD2000; Harvard apparatus) and connected to the catheter. Immediately before the perfusion, the heart was severed and the brain was perfused for 2 min at a flow rate of 2.5 ml/min. All perfusions for IDS and An2-IDS conjugates were performed at a concentration of 5 nM. After perfusion, the brain was briefly perfused with tracer-free solution to wash out the blood vessels for 30 s. At the end of the perfusion, the mice were immediately sacrificed by decapitation and the right hemisphere was isolated on ice and homogenized in Ringer/Hepes buffer before being subjected to capillary depletion.

### Capillary Depletion

The capillary depletion method allows the measure of the accumulation of the perfused molecule into the brain parenchyma by eliminating the binding of tracer to capillaries. The capillary depletion protocol was adapted from the method described by Triguero et al., 1990. A solution of Dextran (35%) was added to the brain homogenate to give a final concentration of 17.5%. After thorough mixing by hand the mixture was centrifuged (10 minutes at 10000 rpm). The resulting pellet contains mainly the capillaries and the supernatant corresponds to the brain parenchyma.

### Determination of Tracer Signal

Aliquots of homogenates, supernatants, pellets and perfusates were taken to measure their contents in radiolabeled molecules. All aliquots were precipitated with trichloroacetic acid in order to get the radiolabeled precipitated protein fractions. [¹²⁵I]-samples samples were counted in a Wizard 1470 Automatic Gamma Counter (Perkin-Elmer Inc, Woodbridge, ON). Results are expressed in term of volume distribution (ml/100 g/2 min) for the different brain compartments.

### Example 7

### GAG Reduction Assay

### Protocol:

### Materials:

Type II MPS Hunter fibroblasts (Coriell institute, GM00298)
Healthy human fibroblasts (Coriell institute, GM05659)
Dulbecco's Modification of Eagle's Medium (DMEM), fetal bovine serum (FBS)
low sulfate Ham's F-12 medium (Invitrogen, catalog #11765-054)
FBS dialysed against 0.15 M NaCl, 10000 Da MWCO (Sigma, catalog # F0392)
³⁵S-sodium sulfate (Perkin-Elmer, catalog # NεX041H002MC)

### Method:

1. MPS II (GM00298) or healthy human fibroblasts (GM05659) in 6-well dishes at 250,000 cells/well in DMEM with 10% fetal bovine serum (FBS).
   Grow for 4 days.
2.—Discard medium, wash cells with warm and sterile PBS.
   Add 1 mL/well of low sulfate F-12 medium with 10% dialysed FBS and 10 μCi ³⁵S-sodium sulfate.
   Add JR-032 or conjugates. Incubate at 37° C., 5% CO₂ for 48 h
3.—Discard medium, wash cells with cold PBS (1 mL, 5 washes).
   Lyse cells in 0.4 mL/well of 1 N NaOH.
   Heat at 60° C. for 60 min to solubilize proteins.
   Remove and aliquot for μBCA protein assay.
4. Count radioactivity with a liquid scintillation counter.
5. microBCA protein assay.
   The data are expressed as ³⁵S CPM per μg protein.

### Results

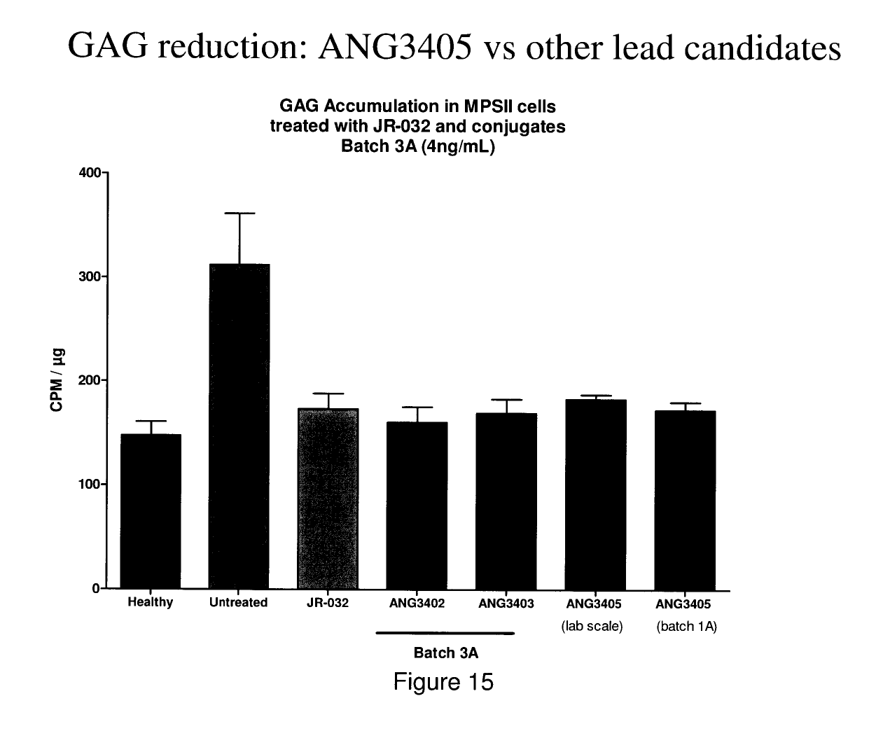

To confirm enzymatic activity with a functional endpoint, the conjugates were assayed for efficacy at reducing GAG levels in fibroblasts from MPSII patients. At a concentration of 4 ng/ml (50 pM), GAG levels are reduced to levels observed in non-disease fibroblasts upon treatment with JR-032, ANG3402, ANG3403, and IDS conjugate 77-18-3 (ANG3405), similar to that observed with JR-032 (see FIG. 15).

### Example 8

### PK/Brain Distribution in Wild Type Mice

### Protocol:

C57Bl6/J (Jackson Laboratories) mice age 12 weeks were dosed with a single iv tail-vein administration of ¹²⁵I enzyme at either 1 mg/kg or 5 mg/kg. Brain and plasma were collected according to the schedule in Table 4 (P: plasma, B: brain). Blood was collected in EDTA coated microtubes (microcuvette capillary Di-Kalium EDTA). Plasma samples were obtained after centrifugation at 5000 rpm for 10 minutes (Beckman Coulter Microfuge 22R Centrifuge). Prior to sacrifice for brain collection, mice were perfused by the heart left ventricle with ice-cold 40 ml saline (5 ml/min, 8 minutes). The brains were collected and weighed (Balance Denver Instrument S-403) in preweighed tubes (Sarstedt 12×75 mm round base). Radioactivity levels in blood plasma (10 μL) and brain were counted by gamma counting on a Wizard 1470 Automatic Gamma Counter (Perkin-Elmer Inc, Woodbridge, ON).

TABLE 4
Plasma and brain collection schedule

| 3 point | | | | | | | | | Total | Total |
|---|---|---|---|---|---|---|---|---|---|---|
| composite | 0.25 | 0.5 | 1 | 4 | 6 | 8 | 24 | 48 | mice* | Drug* |
| Group A | P | P | P & B | | | | | | | |
| Group B | | | | P | P | P&B | | | | |
| Group C | | | | | | | P | P&B | 18 | 2.2 mg |

Mathematical analysis of data was performed using Excel spreadsheets. Raw data was collected as cpm/mg brain and converted to ng/g. For plasma samples, radioactivity counts were converted to μg/ml of plasma. Pharmacokinetic analysis was performed using WinNonlin_Professional version 5.2 (Pharsight Corporation, Mountain View, Calif.).

Data are expressed as mean±S.E.M. All statistical analyses were performed using GraphPad Prism version 5.0 (GraphPad Software Inc., San Diego, Calif.).

### Results

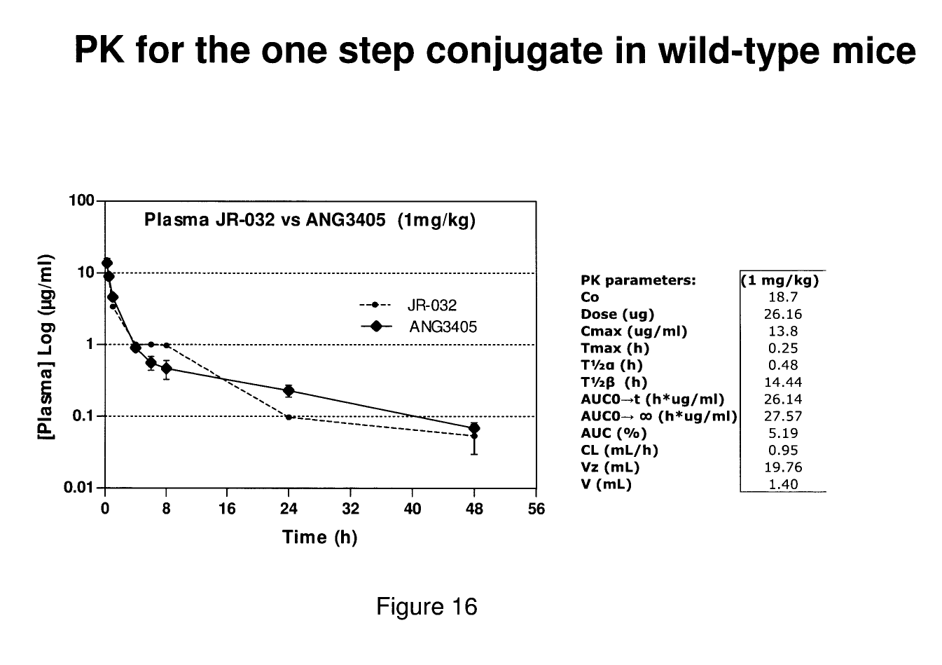

For PK analysis, amount of enzyme in plasma was determined by radioactive counting. Plasma concentration vs. time curves are shown in FIG. 16, with values for analysed parameters in Table 5. For all enzymes, Tmax is observed at 15 minutes. As expected for the conjugates, plasma AUC, and Cmax are lower than for JR-032, while clearance and volume of distribution are higher, consistent with the possibility that the conjugates partition from plasma to tissue more rapidly. The AUC_{%}, or the percent of the AUC∞ that is based on extrapolation, is low for all enzymes, suggesting that greater than 95% of the overall plasma exposure is represented by the AUC₀₋₄₈.

TABLE 5
Plasma PK parameters for JR-032 and conjugates

| | | | | 77-18-3 |
|---|---|---|---|---|
| | JR-032 | ANG3402 | ANG3403 | (ANG3405) |
| Cmax (μg/ml) | 14.4 | 10.0 | 9.7 | 13.8 |
| Tmax (hr) | 0.25 | 0.25 | 0.25 | 0.25 |
| AUC₀₋₄₈ (hr *μg/ml) | 31.6 | 27.0 | 25.9 | 26.14 |
| AUC_{0-∞} (hr*μg/ml) | 32.3 | 27.5 | 26.5 | 27.57 |
| AUC_{%} | 2.4 | 1.9 | 2.0 | 5.19 |
| T1/2β (hr) | 10.1 | 9.1 | 9.3 | 14.44 |
| k (1/hr) | 0.069 | 0.076 | 0.075 | 0.05 |
| Cl (ml/hr) | 0.82 | 1.1 | 1.1 | 0.95 |
| Vd (ml/kg) | 443 | 539 | 544 | 650 |

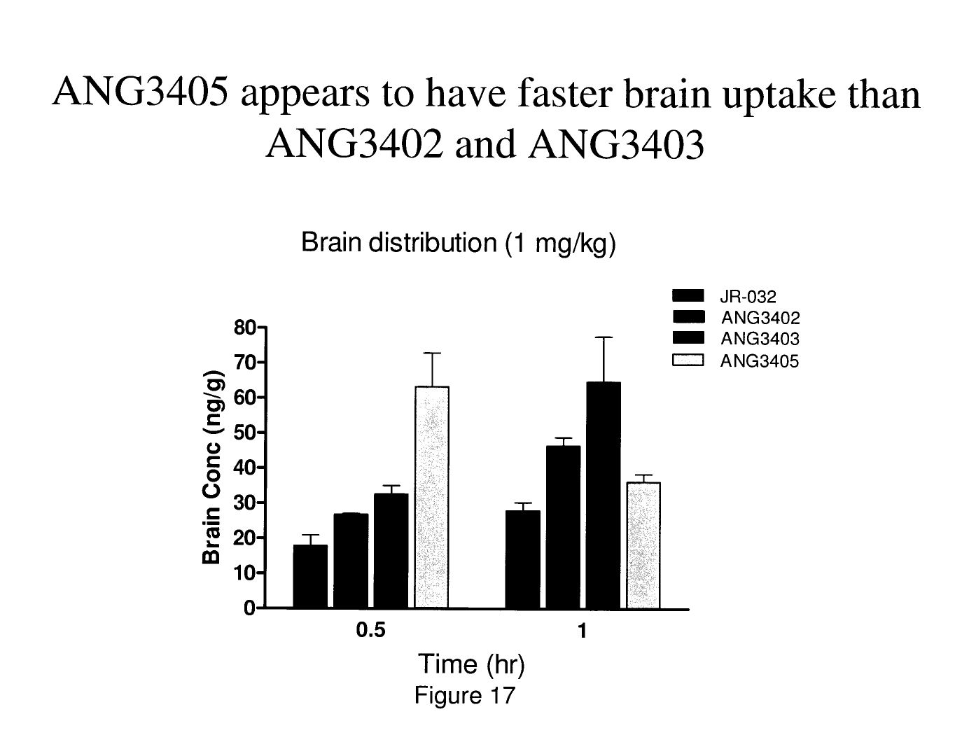

Concentrations of conjugates compared with IDS in the brain at 30 minutes and 1 hour are shown in FIG. 17. 77-18-3 appears to have faster brain uptake than other conjugates tested.

### Example 9

### Effect of Conjugates on GAG Accumulation in the Brain in Hemizygous Iduronate-2-Sulfatase Knock Out Mice

### Protocol

Male hemizygous iduronate-2-sulfatase gene knock-out mice (supplied by Oriental BioService Inc.; Minamiyamashiro Laboratory) aged 18 weeks (on receipt) were dosed at a volume of 5 mL/kg body weight via injection into the caudal vein twice a week for 8 weeks according to Table 6. Male wild type animals (18 weeks old) were used in test group 1 as a control.

TABLE 6
Dosing protocol for GAG accumulation study

| | Type of | Dosage | Dosing | No of |
|---|---|---|---|---|
| Test group | animal | (mg/kg) | frequency | animals |
| 1. Wild type | WT | Not dosed | Not dosed | 5 |
| 2. JR-032 | Hemi | 1 | twice/week | 5 |
| 3. Vehicle | Hemi | 1 | twice/week | 5 |
| 4. ANG3403 | Hemi | 1 | twice/week | 5 |
| 5. ANG3402 | Hemi | 1 | twice/week | 5 |
| 6. ANG3405 | Hemi | 1 | twice/week | 5 |

JR-032, ANG3402, ANG3403, or 77-18-3 were administered in vehicle (20 mM Sodium Phosphate, 137 mM NaCl, pH 6). In test group 3, vehicle only was administered.

One week after completion of the 8 week administration, the auricle of the right atrium was cut open under 20% isoflurane anesthesia and about 30 mL saline was perfused from the left ventricle with a syringe and a needle. After perfusion, the brain (cerebrum and cerebellum) was removed. The brain was divided into the right brain and left brain. The right brain was weighed and frozen and the left brain was immersed in 10% neutral buffered formalin.

The fixed left brains were trimmed sagitally and embedded in paraffin. The paraffin embedded tissue specimens were sectioned using a microtome to get 5 sections on the approx. 0.96+/−0.24 mm lateral site (thickness of sections: 4 μm). Two sections were used for staining with H&E and LAMP-1.

The frozen tissue was freeze-dried (FZ-Compact, Asahi Life Science Co, Ltd.), cut into small pieces, and weighed. 0.5 mol/L tris HCl buffer solution (pH 7.5) containing 50 mg/mL actinase E was added such that the total additive amount of the solution is 1 ml per 100 mg dry weight of the tissues. The mixture was heated at 100° C. for 10 minutes using a dry bath incubator. Additional 0.5 mol/L tris HCl buffer solution (pH 7.5) containing 50 mg/mL actinase E was then added such that the ratio of the dry weight of the tissues to actinase E is 50 mg to 1 mg and the mixture was incubated at 60° C. for about 16 hours using a dry heat sterilizer. The mixture was then heated at 100° C. for 10 minutes using a dry bath incubator followed by centrifugation at 24° C. at about 20400 g for 10 minutes. The supernatant was removed and frozen for more than 12 hours. The supernatant was then thawed at room temperature and centrifuged again at 24° C. at about 20400 g for 10 minutes. The supernatant was removed and frozen.

A Wieslab® sGAG quantitative kit (EURO-DIAGNOSTICA) was used to determine GAG concentrations twice in 50 μl samples from brain, in a 50 μl blank sample (water for injection) and in 50 μl calibration samples (solutions of chondroitin sulfate B sodium salt in water for injection at concentrations of 640 μg/ml, 320 μg/ml, 160 μg/ml, 80 μg/ml 40 μg/ml and 20 μg/ml).

Briefly, 50 μl of 8 M guanidine-HCl was added to each sample and the mixture was allowed to react at room temperature for 15 minutes. 50 μl of SAT solution (0.3% H₂SO₄ and 0.75% Triton X-100) was then added and the mixture was allowed to react at room temperature for 15 minutes. 750 μl of Alcian Blue working solution (prepared by mixing water for injection, the SAT solution and Alcian Blue stock solution (0.1% H₂SO₄ and 0.4 M guanidine HCl) at a ratio of 9:5:1) was added to each sample and the mixture was allowed to react at room temperature for 15 minutes. The samples were then centrifuged at 12600 g for 15 minutes at 24° C. and the supernatant was discarded. 500 μl of DMSO solution (40% dimethylsulphoxide and 0.05 M MgCl₂) was added to the tube and the contents of the tube were stirred with a mixer at room temperature for 15 minutes followed by centrifugation at 12600 g for 15 minutes at 24° C. The supernatant was discarded and 500 μl Gu-Prop (4 M guanidine-HCl, 33% 1-propanol and 0.25% Triton X-100) was added to each sample. The tubes were stirred with a mixer at room temperature for 15 minutes so that precipitates were completely dissolved. 200 μl of each sample was dispensed into wells in a 96 well microplate. A microplate reader was used to obtain absorbance values at a wavelength of 600 nm. GAG concentrations were calculated by a linear method with analysis software (KC4 v3.4 DS Pharma Biomedical Co., Ltd.). Mean values (μg/ml) of 2 measurements of GAG concentrations were calculated.

The accuracy of GAG concentrations of the calibration samples were calculated. When the accuracy and correlation coefficient were not within the evaluation criteria, GAG concentrations in the tissue samples were not calculated. (Evaluation criteria for accuracy: coefficient of variation is within 15% (within 20% for the 20 μg/ml sample). Evaluation criteria for correlation coefficient: 0.997 or higher (rounded to 4 decimal places)).

The GAG concentrations measured in the brain samples were converted to the concentration in the dry weight of brain by the following formula:

$$[ GAG ] d = \frac{[ GAG ] s \times A}{W}$$

[GAG]d=GAG concentration in dry tissue (μg/mg)
[GAG]s=GAG concentration in sample (μg/ml)
A=Additive amount (ml)
W=Dry tissue weight (mg)

### Results

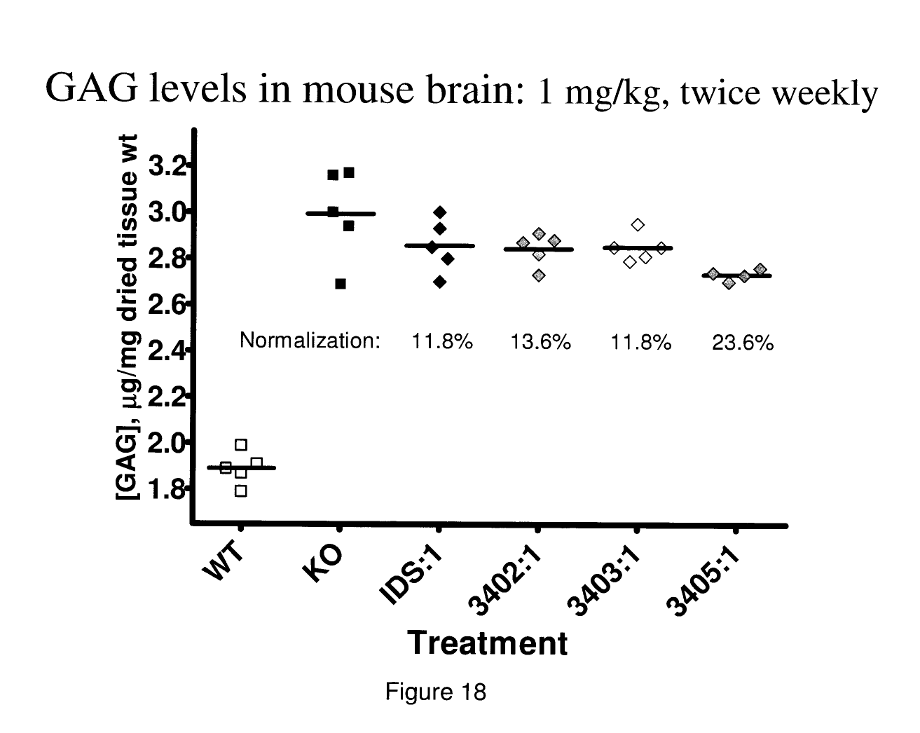

No significant difference in GAG concentration was observed in brain tissue in hemizygous mice dosed with enzyme alone, ANG3402, or ANG3403, however, at 1 mg/kg twice weekly, 77-18-3 resulted in a 2-fold reduction over the enzyme alone. (see FIG. 18).

### Other Embodiments

All patents, patent applications, and publications mentioned in this specification are herein incorporated by reference including PCT Application No. PCT/CA2012/050865, filed Nov. 30, 2012, to the same extent as if each independent patent, patent application, or publication was specifically and individually indicated to be incorporated by reference.

What is claimed is:

1. A compound including the amino acid sequence of formula (I):
X1-X2-X3-X4-X5-X6-X7-X8-X9-X10-X11-X12-X13-X14-X15-X16-X17-X18-X19 Formula (I)
wherein X1-X19 are, independently, any amino acid or absent provided that at least one amino acid is N_{ε}-protected lysine; wherein the amino acid sequence has at least 70% identity to any one of SEQ ID NO: 1-69, 71-73, 75-105 and 107-216; and
wherein said amino acid sequence contains a single reactive primary amine group.

2-6. (canceled)

7. The compound of claim 1, wherein at least one amino acid residue is Nε-(acetyl)-L-lysine.

8. The compound of claim 1, wherein said amino acid sequence has at least 80% identity to SEQ ID NO:97.

9. The compound of claim 1, herein said amino acid sequence has 100% identity to SEQ ID NO:97.

10. A compound having the formula: 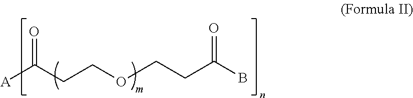
wherein A is an enzyme, an enzyme fragment that retains the activity of said enzyme, an enzyme analog, wherein said enzyme, enzyme fragment, or enzyme analog that exhibits enzyme activity is attached via one or more NH groups derived from the reaction of a primary amine group;
m is an integer between 1 and 45;
n is an integer between 1 and 6; and
B comprises an amino acid sequence that contains a single reactive primary amine group that has either (a) at least 70% identity to any one of SEQ ID NO: 1-69, 71-73, 75-105 and 107-216, or (b) the amino acid sequence of any one of formulae I, Ia, Ib, Ic, Id or Ie;
wherein B is attached via an NH group derived from reaction of its single reactive primary amine group; and
wherein the compound exhibits the activity of said enzyme.

11-25. (canceled)

26. A population of the compounds of claim 10, wherein the average value of n is 1 to 3.

27-29. (canceled)

30. A compound having the formula: 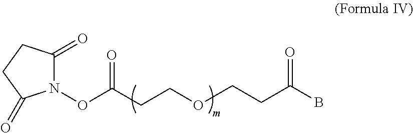
wherein m is 1 to 45; and
B comprises an amino acid sequence that contains a single reactive primary amine group that has either (a) at least 70% identity to any one of SEQ ID NO: 1-69, 71-73, 75-105 and 107-216, or (b) the amino acid sequence of any one of formulae I, Ia, Ib, Ic, Id or Ie; and
wherein B is attached via an NH group derived from reaction of its single reactive primary amine group.

31. The compound of claim 30, having the structure:

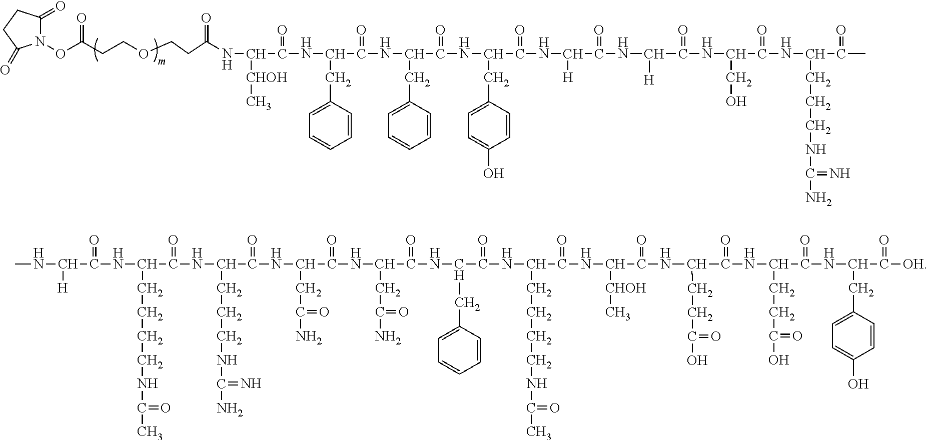

32. The compound or population of claim 30, wherein m is 2, 4, 7, 9, or 13.

33. The compound of claim 30, wherein one or more amino acids are substituted with the corresponding D-amino acid.

34. The compound of claim 33, wherein four amino acids are substituted with the corresponding D-amino acid.

35. The compound of claim 31, wherein Arg⁸, Lys¹⁰, Arg¹¹, and/or Lys¹⁵ are substituted with the corresponding D-amino acid.

36. The compound of claim 1, wherein said compound is efficiently transported to the lysosome.

37. The compound claim 1, wherein said compound is efficiently transported across the blood brain barrier.

38. A composition comprising one or more nanoparticles, wherein said nanoparticle is conjugated to a compound of claim 1.

39. A composition comprising a liposome formulation of a compound of claim 1.

40. A pharmaceutical composition comprising a compound a compound of claim 10 and a pharmaceutically acceptable carrier.

41. A method of treating or treating prophylactically mucopolysaccharidosis Type II (MPS-II), said method comprising administering to a subject in need thereof a compound of claim 10.

42-48. (canceled)

49. A method of producing an enzyme conjugate, said method comprising reacting a compound of formula IV: 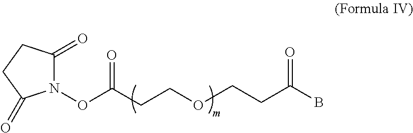
with an enzyme, an enzyme fragment that retains the activity of said enzyme, or an enzyme analog, wherein said enzyme, enzyme fragment, or enzyme analog has one or more primary amine groups, under conditions to produce a compound of formula II: 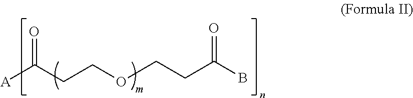
wherein A is said enzyme, enzyme fragment, or enzyme analog, and said enzyme, fragment, or analog is attached via one or more NH groups derived from reaction of a primary amine group;
m is 1 to 45;
n is 1 to 6; and
B comprises an amino acid sequence that contains a single reactive primary amine group that has either (a) at least 70% identity to any one of SEQ ID NO: 1-69, 71-73, 75-105 and 107-216, or (b) the amino acid sequence of any one of formulae I, Ia, Ib, Ic, Id or Ie; and
wherein B is attached via an NH group derived from reaction of its single reactive primary amine group.

50-54. (canceled)
